(19) United States
(12) Patent Application Publication
(10) Pub. No.: US 2021/0108170 A1
(43) Pub. Date: Apr. 15, 2021

(54) PHENOTYPIC READOUT FOR EDITED SEQUENCES IN LIVE CELLS IN AUTOMATED INSTRUMENTATION

(71) Applicant: Inscripta, Inc., Boulder, CO (US)

(72) Inventor: Stephen Tanner, Boulder, CO (US)

(21) Appl. No.: 17/069,802

(22) Filed: Oct. 13, 2020

Related U.S. Application Data
(60) Provisional application No. 62/914,952, filed on Oct. 14, 2019.

(51) Int. Cl. C12M 1/36; G06T 7/00; C12N 9/22; G01N 33/52; C12M 1/00; C12M 3/00

(52) U.S. Cl. CPC C12M 41/48; G06T 7/0012; C12M 23/44; G01N 33/52; C12M 47/04; C12N 9/22

(57) ABSTRACT

The present disclosure provides instruments, modules and methods for phenotypic readout for edited cells following nucleic acid-guided nuclease genome editing. The disclosure provides improved automated instruments that perform methods—including high throughput methods—for screening cells that have been subjected to editing and identifying cells that have been properly edited and display a desired phenotype.

### RELATED CASES

This application claims priority to U.S. Ser. No. 62/914,952, filed 14 Oct. 2019, entitled “Phenotypic Readout for Edited Sequences in Live Cells in Automated Instrumentation.”

### FIELD OF THE INVENTION

This invention relates to improved instruments, modules, and methods to screen and select desired edits in live cells.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate, that the methods referenced herein do not constitute prior art under the applicable statutory provisions.

The ability to make precise, targeted changes to the genome of living cells has been a long-standing goal in biomedical research and development. Recently, various nucleases have been identified that allow for manipulation of gene sequences, and hence gene function. The nucleases include nucleic acid-guided nucleases, which enable researchers to generate permanent edits in live cells. Although instrumentation exists for automated gene editing (see, co-owned U.S. Pat. No. 10,253,316, issued 9 Apr. 2019; U.S. Pat. No. 10,329,559, issued 25 Jun. 2019; U.S. Pat. No. 10,323,242, issued 18 Jun. 2019; U.S. Pat. No. 10,421,959, issued 24 Sep. 2019; U.S. Pat. No. 10,465,185, issued 5 Nov. 2019; U.S. Pat. No. 10,519,437, issued 31 Dec. 2019; U.S. Pat. No. 10,584,333, issued 10 Mar. 2020; U.S. Pat. No. 10,584,334, issued 10 Mar. 2020; U.S. Pat. No. 10,647,982, issued 12 May 2020; U.S. Pat. No. 10,689,645, issued 23 Jun. 2020; U.S. Pat. No. 10,738,301, issued 11 Aug. 2020; and U.S. Ser. No. 16/920,853, filed 6 Jul. 2020; and Ser. No. 16/988,694, filed 9 Aug. 2020, all of which are herein incorporated by reference in their entirety), to date there are no methods or instrumentation that allow for automated growth, editing and phenotypic identification of edited cells, The present invention satisfies this need.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following Detailed Description including those aspects illustrated in the accompanying drawings and defined in the appended claims.

The present disclosure provides instruments, modules and methods to enable automated high-throughput and sensitive phenotypic screening to identify edited cells in populations of cells that have been subjected to nucleic acid-guided nuclease editing. The instruments, modules, and methods take advantage of isolation or substantial isolation, where the term “isolation” in this context refers to the process of separating cells and growing them into clonally-isolated formats. The term “substantial isolation” refers to the process of separating cells in a population of cells into “groups” of 2 to 100, or 2 to 50, and preferably 2 to 10 cells. Isolation (or substantial isolation), followed by an initial period of growth (e.g., incubation), editing, and imaging leads to enrichment of edited cells.

One particularly facile module or device for isolation or substantial isolation, editing and imaging is a solid wall device where cells are substantially isolated, grown in a clonal (or substantially clonal) format, edited, and imaged. Solid wall devices or modules and the uses thereof are described in detail herein. Certain embodiments of the instruments, modules, and methods provide for enriching for edited cells during nucleic acid-guided nuclease editing, where the methods comprise transforming cells with one or more vectors comprising a promoter driving expression of a nuclease, a promoter driving transcription of a guide nucleic acid and a donor DNA sequence; diluting the transformed cells to a cell concentration sufficient to substantially isolate the transformed cells on a substrate; growing (e.g., incubating) the substantially isolated cells on the substrate; providing conditions for editing; and imaging the cells after a screening reagent is introduced into the solid wall device in which the edited cells are growing. In some aspects at least the gRNA is optionally under the control of an inducible promoter.

These aspects and other features and advantages of the invention are described below in more detail.

### BRIEF DESCRIPTION OF THE FIGURES

FIG. 1 is a simplified flow chart of an exemplary method for enriching and selecting cells with desired edits by phenotypic screening.

FIG. 2A depicts a simplified graphic of a workflow for isolating, editing and normalizing cells in a solid wall device. 2B depicts a simplified graphic of a workflow variation for substantially isolating, editing and normalizing cells in a solid wall device.

FIGS. 3A-3H depict a different embodiment of a SWIIN module, where the retentate and permeate members are coincident with reservoir assembly. FIG. 3I depicts the embodiment of the SWIIN module in FIGS. 3A-3H further comprising a heater and a heated cover. FIG. 3J is an exemplary pneumatic architecture diagram for the SWIIN module described in relation to FIGS. 3A-3H, with the status of the components for the various steps listed in Tables 1-3.

FIGS. 4A-4D depict a stand-alone, integrated, automated multi-module instrument and components thereof, including an isolation module, with which to generate and identify edited cells.

FIG. 5A depicts one embodiment of a rotating growth vial for use with a cell growth module described herein. FIG. 5B illustrates a perspective view of one embodiment of a rotating growth device in a cell growth module housing. FIG. 5C depicts a cut-away view of the cell growth module from FIG. 5B. FIG. 5D illustrates the cell growth module of FIG. 5B coupled to LED, detector, and temperature regulating components.

FIG. 6A is a model of tangential flow filtration employed by the TFF device presented herein. FIG. 6B depicts a top view of a lower member of one embodiment of an exemplary TFF device. FIG. 6C depicts a top view of upper and lower members and a membrane of an exemplary TFF device. FIG. 6D depicts a bottom view of upper and lower members and a membrane of an exemplary TFF device. FIGS. 6E-6K depict various views of yet another embodiment of a TFF module having fluidically coupled reservoirs. FIG. 6L is an exemplary pneumatic architecture diagram for the TFF module described in relation to FIGS. 6E-6K.

FIGS. 7A and 7B are top perspective and bottom perspective views, respectively, of flow-through electroporation devices (here, there are six such devices co-joined). FIG. 7C is a top view of one embodiment of an exemplary flow-through electroporation device. FIG. 7D depicts a top view of a cross section of the electroporation device of FIG. 7C. FIG. 7E is a side view cross section of a lower portion of the electroporation devices of FIGS. 7C and 7D.

FIG. 8 is a simplified block diagram of an embodiment of an exemplary automated multi-module cell processing instrument comprising an isolation or substantial isolation/incubation/editing and imaging module.

### DETAILED DESCRIPTION

All of the functionalities described in connection with one embodiment are intended to be applicable to the additional embodiments described herein except where expressly stated or where the feature or function is incompatible with the additional embodiments. For example, where a given feature or function is expressly described in connection with one embodiment but not expressly mentioned in connection with an alternative embodiment, it should be understood that the feature or function may be deployed, utilized, or implemented in connection with the alternative embodiment unless the feature or function is incompatible with the alternative embodiment.

The practice of the techniques described herein may employ, unless otherwise indicated, conventional techniques and descriptions of organic chemistry, polymer technology, molecular biology (including recombinant techniques), cell biology, biochemistry, and sequencing technology, which are within the skill of those who practice in the art. Such conventional techniques include polymer array synthesis, hybridization and ligation of polynucleotides, and detection of hybridization using a label. Specific illustrations of suitable techniques can be had by reference to the examples herein. However, other equivalent conventional procedures can, of course, also be used. Such conventional techniques and descriptions can be found in standard laboratory manuals such as Green, et al., eds., Genome Analysis: A Laboratory Manual Series (Vols. I-IV) (1999); Weiner, Gabriel, Stephens, eds., Genetic Variation: A Laboratory Manual (2007); Dieffenbach, Dveksler, eds., PCR Primer: A Laboratory Manual (2003); Bowtell and Sambrook, DNA Microarrays: A Molecular Cloning Manual (2003); Mount, Bioinformatics: Sequence and Genome Analysis (2004); Sambrook and Russell, Condensed Protocols from Molecular Cloning: A Laboratory Manual (2006); Stryer, Biochemistry (4th Ed.) W.H. Freeman, New York N.Y. (1995); Gait, “Oligonucleotide Synthesis: A Practical Approach” (1984), IRL Press, London; Nelson and Cox, Lehninger, Principles of Biochemistry 3^{rd} Ed., W. H. Freeman Pub., New York, N.Y. (2000); Berg et al., Biochemistry, 5^{th} Ed., W.H. Freeman Pub., New York, N.Y. (2002); Doyle & Griffiths, eds., Cell and Tissue Culture: Laboratory Procedures in Biotechnology, Doyle & Griffiths, eds., John Wiley & Sons (1998); G. Hadlaczky, ed. Mammalian Chromosome Engineering—Methods and Protocols, Humana Press (2011); and Lanza and Klimanskaya, eds., Essential Stem Cell Methods, Academic Press (2011), all of which are herein incorporated in their entirety by reference for all purposes. Fluorescence and imaging techniques may be found in Valeur, et al., Molecular Fluorescence: Principles and Applications, 2^{nd} Ed., Wiley Network (2012); Handbook of Fluorescence Spectroscopy and Imaging: From Ensemble to Single Molecules, Sauer, Wiley Press (2010); and Introduction to Fluorescence Spectroscopy, Schulman, Wiley-Interscience (1999), all of which are incorporated herein in their entirety by reference for all purposes. CRISPR-specific techniques can be found in, e.g., Appasani and Church, Genome Editing and Engineering from TALENs and CRISPRs to Molecular Surgery (2018); and Lindgren and Charpentier, CRISPR: Methods and Protocols (2015); both of which are herein incorporated in their entirety by reference for all purposes.

Note that as used herein and in the appended claims, the singular forms “a,” “an,” and “the” include plural referents unless the context clearly dictates otherwise. Thus, for example, reference to “a cell” refers to one or more cells, and reference to “the system” includes reference to equivalent steps, methods and devices known to those skilled in the art, and so forth. Additionally, it is to be understood that terms such as “left,” “right,” “top,” “bottom,” “front,” “rear,” “side,” “height,” “length,” “width,” “upper,” “lower,” “interior,” “exterior,” “inner,” and/or “outer” that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Additionally, the terms “approximately,” “proximate,” “minor,” and similar terms generally refer to ranges that include the identified value within a margin of 20%, 10% or preferably 5% in certain embodiments, and any values therebetween.

Unless defined otherwise, all technical and scientific terms used herein have the same meaning as commonly understood by one of ordinary skill in the art to which this invention belongs. All publications mentioned herein are incorporated by reference for the purpose of describing and disclosing devices, methods and cell populations that may be used in connection with the presently described invention.

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either both of those included limits are also included in the invention.

In the following description, numerous specific details are set forth to provide a more thorough understanding of the present invention. However, it will be apparent to one of ordinary skill in the art that the present invention may be practiced without one or more of these specific details. In other instances, features and procedures well known to those skilled in the art have not been described in order to avoid obscuring the invention.

The term “complementary” as used herein refers to Watson-Crick base pairing between nucleotides and specifically refers to nucleotides that are hydrogen bonded to one another with thymine or uracil residues linked to adenine residues by two hydrogen bonds and cytosine and guanine residues linked by three hydrogen bonds. In general, a nucleic acid includes a nucleotide sequence described as having a “percent complementarity” or “percent homology” to a specified second nucleotide sequence. For example, a nucleotide sequence may have 80%, 90%, or 100% complementarity to a specified second nucleotide sequence, indicating that 8 of 10, 9 of 10 or 10 of 10 nucleotides of a sequence are complementary to the specified second nucleotide sequence. For instance, the nucleotide sequence 3′-TCGA-5′ is 100% complementary to the nucleotide sequence 5′-AGCT-3′; and the nucleotide sequence 3′-TCGA-5′ is 100% complementary to a region of the nucleotide sequence 5′-TTAGCTGG-3′.

The term DNA “control sequences” refers collectively to promoter sequences, polyadenylation signals, transcription termination sequences, upstream regulatory domains, origins of replication, internal ribosome entry sites, nuclear localization sequences, enhancers, and the like, which collectively provide for the replication, transcription and translation of a coding sequence in a recipient cell. Not all of these types of control sequences need to be present so long as a selected coding sequence is capable of being replicated, transcribed and—for some components—translated in an appropriate host cell.

As used herein the term “donor DNA” or “donor nucleic acid” refers to nucleic acid that is designed to introduce a DNA sequence modification (insertion, deletion, substitution) into a locus by homologous recombination using nucleic acid-guided nucleases. For homology-directed repair, the donor DNA must have sufficient homology to the regions flanking the “cut site” or site to be edited in the genomic target sequence. The length of the homology arm(s) will depend on, e.g., the type and size of the modification being made. For example, the donor DNA will have at least one region of sequence homology (e.g., one homology arm) to the genomic target locus. In many instances and preferably, the donor DNA will have two regions of sequence homology (e.g., two homology arms) to the genomic target locus. Preferably, an “insert” region or “DNA sequence modification” region—the nucleic acid modification that one desires to be introduced into a genome target locus in a cell—will be located between two regions of homology. The DNA sequence modification may change one or more bases of the target genomic DNA sequence at one specific site or multiple specific sites. A change may include changing 1, 2, 3, 4, 5, 10, 15, 20, 25, 30, 35, 40, 50, 75, 100, 150, 200, 300, 400, or 500 or more base pairs of the target sequence. A deletion or insertion may be a deletion or insertion of 1, 2, 3, 4, 5, 10, 15, 20, 25, 30, 35, 40, 50, 75, 100, 150, 200, 300, 400, or 500 or more base pairs of the target sequence. The donor DNA optionally further includes an alteration to the target sequence, e.g., a PAM mutation, which prevents binding of the nuclease at the PAM or spacer in the target sequence after editing has taken place.

As used herein, “enrichment” refers to enriching for edited cells by isolation or substantial isolation of cells, initial growth of cells into cell colonies (e.g., incubation), editing (optionally induced, particularly in bacterial systems), and growing the cell colonies into terminal-sized colonies (e.g., saturation or normalization of colony growth). As used herein, “cherry picking” or “selection of edited cells” refers to the process of using a combination of isolation or substantial isolation, initial growth of cells into colonies (incubation), editing (optionally induced, particularly in bacterial systems), then using cell growth—measured by colony size, concentration of metabolites or waste products, or other characteristics that correlate with the rate of growth of the cells—to select for cells that have been edited based on editing-induced growth delay. Selection may entail picking or selecting slow-growing cell colonies; alternatively, selection may entail eliminating (by, e.g., eradicating or removing) the faster-growing cell colonies.

The terms “guide nucleic acid” or “guide RNA” or “gRNA” refer to a polynucleotide comprising 1) a guide sequence capable of hybridizing to a genomic target locus, and 2) a scaffold sequence capable of interacting or complexing with a nucleic acid-guided nuclease.

“Homology” or “identity” or “similarity” refers to sequence similarity between two peptides or, more often in the context of the present disclosure, between two nucleic acid molecules. The term “homologous region” or “homology arm” refers to a region on the donor DNA with a certain degree of homology with the target genomic DNA sequence. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences.

As used herein, the terms “isolation” or “isolate” mean to separate individual cells so that each cell (and the colonies formed from each cell) will be separate from other cells; for example, a single cell in a single microwell, or 100 single cells each in its own microwell. “Isolation” or “isolated cells” result in one embodiment, from a Poisson distribution in arraying cells. The terms “substantially isolated”, “largely isolated”, and “substantial isolation” mean cells are largely separated from one another, in small groups or batches. That is, when 2, 3, 4, 5, 6, 7, 8, 9, 10, 11, 12, 13, 14, 15, 16, 17, 18, 19, 20, 21, 22, 23, 24, 25, 26, 27, 28, 29, 30 or up to 50—but preferably 10 or less cells—are delivered to a microwell. “Substantially isolated” or “largely isolated” result, in one embodiment, from a “substantial Poisson distribution” in arraying cells. With more complex libraries of edits—or with libraries that may comprise lethal edits or edits with greatly-varying fitness effects—it is preferred that cells be isolated via a Poisson distribution.

“Operably linked” refers to an arrangement of elements where the components so described are configured so as to perform their usual function. Thus, control sequences operably linked to a coding sequence are capable of effecting the transcription, and in some cases, the translation, of a coding sequence. The control sequences need not be contiguous with the coding sequence so long as they function to direct the expression of the coding sequence. Thus, for example, intervening untranslated yet transcribed sequences can be present between a promoter sequence and the coding sequence and the promoter sequence can still be considered “operably linked” to the coding sequence. In fact, such sequences need not reside on the same contiguous DNA molecule (i.e. chromosome) and may still have interactions resulting in altered regulation.

A “promoter” or “promoter sequence” is a DNA regulatory region capable of binding RNA polymerase and initiating transcription of a polynucleotide or polypeptide coding sequence such as messenger RNA, ribosomal RNA, small nuclear or nucleolar RNA, guide RNA, or any kind of RNA transcribed by any class of any RNA polymerase I, II or III. Promoters may be constitutive or inducible. In the methods described herein optionally the promoters driving transcription of the gRNAs is inducible.

As used herein the term “screening agent” refers to an agent that allows one to identify edited cells with a desired phenotype. One type of screening agent of particular interest herein is a fluorescent screening agent.

As used herein the term “selectable marker” refers to a gene introduced into a cell, which confers a trait suitable for artificial selection. General use selectable markers are well-known to those of ordinary skill in the art. Drug selectable markers such as ampicillin/carbenicillin, kanamycin, chloramphenicol, erythromycin, tetracycline, gentamicin, bleomycin, streptomycin, puromycin, hygromycin, blasticidin, and G418 may be employed. In other embodiments, selectable markers include, but are not limited to human nerve growth factor receptor (detected with a MAb, such as described in U.S. Pat. No. 6,365,373); truncated human growth factor receptor (detected with MAb); mutant human dihydrofolate reductase (DHFR; fluorescent MTX substrate available); secreted alkaline phosphatase (SEAP; fluorescent substrate available); human thymidylate synthase (TS; confers resistance to anti-cancer agent fluorodeoxyuridine); human glutathione S-transferase alpha (GSTA1; conjugates glutathione to the stem cell selective alkylator busulfan; chemoprotective selectable marker in CD34+cells); CD24 cell surface antigen in hematopoietic stem cells; rhamnose; human CAD gene to confer resistance to N-phosphonacetyl-L-aspartate (PALA); human multi-drug resistance-1 (MDR-1; P-glycoprotein surface protein selectable by increased drug resistance or enriched by FACS); human CD25 (IL-2α; detectable by MAb-FITC); Methylguanine-DNA methyltransferase (MGMT; selectable by carmustine); and Cytidine deaminase (CD; selectable by Ara-C). “Selective medium” as used herein refers to cell growth medium to which has been added a chemical compound or biological moiety that selects for or against selectable markers.

The terms “target genomic DNA sequence”, “target sequence”, or “genomic target locus” refer to any locus in vitro or in vivo, or in a nucleic acid (e.g., genome) of a cell or population of cells, in which a change of at least one nucleotide is desired using a nucleic acid-guided nuclease editing system. The target sequence can be a genomic locus or extrachromosomal locus.

A “vector” is any of a variety of nucleic acids that comprise a desired sequence or sequences to be delivered to and/or expressed in a cell. Vectors are typically composed of DNA, although RNA vectors are also available. Vectors include, but are not limited to, plasmids, fosmids, phagemids, virus genomes, YACs, BACs, mammalian synthetic chromosomes, and the like. As used herein, the phrase “engine vector” comprises a coding sequence for a nuclease—optionally under the control of an inducible promoter—to be used in the nucleic acid-guided nuclease systems and methods of the present disclosure. The engine vector may also comprise, in a bacterial system, the λ Red recombineering system or an equivalent thereto, as well as a selectable marker. As used herein the phrase “editing vector” comprises a donor nucleic acid, including an alteration to the target sequence which prevents nuclease binding at a PAM or spacer in the target sequence after editing has taken place, and a coding sequence for a gRNA optionally under the control of an inducible promoter (and preferably under the control of an inducible promoter in bacterial systems). The editing vector may also comprise a selectable marker and/or a barcode. In some embodiments, the engine vector and editing vector may be combined; that is, the contents of the engine vector may be found on the editing vector.

### Editing in Nucleic Acid-Guided Nuclease Genome Systems Generally

The present disclosure provides instruments, modules and methods for nucleic acid-guided nuclease genome editing that provide improvement in screening for and detecting cells whose genomes have been properly edited via imaging. Presented herein are methods that take advantage of isolation (separating cells and growing them into clonal colonies) and imaging of the growing colonies using fluorescent screening agents. The instruments, modules, and methods may be applied to all cell types including, archaeal, prokaryotic, and eukaryotic (e.g., yeast, fungal, plant and animal) cells.

The instruments, modules, and methods described herein employ editing cassettes comprising a guide RNA (gRNA) sequence covaiently linked to a donor DNA sequence where, particularly in bacterial systems, the gRNA optionally is under the control of an inducible promoter (e.g., the editing cassettes are CREATE cassettes; see U.S. Pat. Nos. 10,240,167; 10,266,849; 9,982,278; 10,351,877; 10,364,442; 10,435,715; 10,465,207; 10,669,559; 10,711,284; and 10,713,180; and U.S. Ser. No. 16/550,092, filed 23 Aug. 2019; and Ser. No. 16/938,739, filed 24 Jul. 2020, all of which are incorporated by reference herein, all of which are incorporated by reference in their entirety). The disclosed methods allow for cells to be transformed, substantially isolated, grown for several doublings (e.g., incubation), after which editing is allowed. The isolation process effectively negates the effect of unedited cells taking over the cell population. The edited cells can then be imaged to identify cells with a desired phenotype. Additionally, the methods may be leveraged to create iterative editing systems to generate combinatorial libraries of cells with two to many edits in each cellular genome. Optionally using inducible gRNA constructs (and in some embodiments, inducible nuclease constructs) provides “pulsed” exposure of the cells to active editing components, which 1) allows for the cells to be arrayed (e.g., largely isolated) prior to initiation of the editing procedure, 2) decreases off-target activity, and 3) allows for identification of rare cell edits.

The instruments, compositions and methods described herein improve editing systems in which nucleic acid-guided nucleases (e.g., RNA-guided nucleases) are used to edit specific target regions in an organism's genome. A nucleic acid-guided nuclease complexed with an appropriate synthetic guide nucleic acid in a cell can cut the genome of the cell at a desired location. The guide nucleic acid helps the nucleic acid-guided nuclease recognize and cut the DNA at a specific target sequence. By manipulating the nucleotide sequence of the guide nucleic acid, the nucleic acid-guided nuclease may be programmed to target any DNA sequence for cleavage as long as an appropriate protospacer adjacent motif (PAM) is nearby. In certain aspects, the nucleic acid-guided nuclease editing system may use two separate guide nucleic acid molecules that combine to function as a guide nucleic acid, e.g., a CRISPR RNA (crRNA) and trans-activating CRISPR RNA (tracrRNA). In other aspects, the guide nucleic acid may be a single guide nucleic acid that includes both the crRNA and tracrRNA sequences or a single guide nucleic acid that does not require a tracrRNA.

In general, a guide nucleic acid (e.g., gRNA) complexes with a compatible nucleic acid-guided nuclease and can then hybridize with a target sequence, thereby directing the nuclease to the target sequence. A guide nucleic acid can be DNA or RNA; alternatively, a guide nucleic acid may comprise both DNA and RNA. In some embodiments, a guide nucleic acid may comprise modified or non-naturally occurring nucleotides. In cases where the guide nucleic acid comprises RNA, the gRNA is encoded by a DNA sequence on a polynucleotide molecule such as a plasmid, linear construct, or resides within an editing cassette and is optionally-particularly in bacterial systems-under the control of an inducible promoter.

A guide nucleic acid comprises a guide sequence, where the guide sequence is a polynucleotide sequence having sufficient complementarity with a target sequence to hybridize with the target sequence and direct sequence-specific binding of a complexed nucleic acid-guided nuclease to the target sequence. The degree of complementarity between a guide sequence and the corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences. In some embodiments, a guide sequence (the portion of the guide nucleic acid that hybridizes with the target sequence) is about or more than about 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20 nucleotides in length. Preferably the guide sequence is 10-30 or 15-20 nucleotides long, or 15, 16, 17, 18, 19, or 20 nucleotides in length.

In the present methods and compositions, the guide nucleic acid is provided as a sequence to be expressed from a plasmid or vector and comprises both the guide sequence and the scaffold sequence as a single transcript. Alternatively, the guide nucleic acids may be transcribed from two separate sequences. The guide nucleic acid can be engineered to target a desired target DNA sequence by altering the guide sequence so that the guide sequence is complementary to the target DNA sequence, thereby allowing hybridization between the guide sequence and the target DNA sequence. In general, to generate an edit in the target DNA sequence, the gRNA/nuclease complex binds to a target sequence as determined by the guide RNA, and the nuclease recognizes a protospacer adjacent motif (PAM) sequence adjacent to the target sequence. The target sequence can be any polynucleotide (either DNA or RNA) endogenous or exogenous to a prokaryotic or eukaryotic cell, or in vitro. For example, the target sequence can be a polynucleotide residing in the nucleus of a eukaryotic cell. A target sequence can be a sequence encoding a gene product (e.g., a protein) and/or a non-coding sequence (e.g., a regulatory polynucleotide, an intron, a PAM, or “junk” DNA).

The guide nucleic acid may be part of an editing cassette that encodes the donor nucleic acid; that is, the editing cassette may be a CREATE cassette (see, e.g., U.S. Pat. No. 10,253,316, issued 9 Apr. 2019; U.S. Pat. No. 10,329,559, issued 25 Jun. 2019; U.S. Pat. No. 10,323,242, issued 18 Jun. 2019; U.S. Pat. No. 10,421,959, issued 24 Sep. 2019; U.S. Pat. No. 10,465,185, issued 5 Nov. 2019; U.S. Pat. No. 10,519,437, issued 31 Dec. 2019; U.S. Pat. No. 10,584,333, issued 10 Mar. 2020; U.S. Pat. No. 10,584,334, issued 10 Mar. 2020; U.S. Pat. No. 10,647,982, issued 12 May 2020; U.S. Pat. No. 10,689,645, issued 23 Jun. 2020; U.S. Pat. No. 10,738,301, issued 11 Aug. 2020; and U.S. Ser. No. 16/920,853, filed 6 Jul. 2020; and Ser. No. 16/988,694, filed 9 Aug. 2020, all of which are incorporated by reference in their entirety). The guide nucleic acid and the donor nucleic acid may be and typically are under the control of a single (optionally inducible) promoter. Alternatively, the guide nucleic acid may not be part of the editing cassette and instead may be encoded on the engine or editing vector backbone. For example, a sequence coding for a guide nucleic acid can be assembled or inserted into a vector backbone first, followed by insertion of the donor nucleic acid. In other cases, the donor nucleic acid can be inserted or assembled into a vector backbone first, followed by insertion of the sequence coding for the guide nucleic acid. In yet other cases, the sequence encoding the guide nucleic acid and the donor nucleic acid (inserted, for example, in an editing cassette) are simultaneously but separately inserted or assembled into a vector. In yet other embodiments and preferably, the sequence encoding the guide nucleic acid and the sequence encoding the donor nucleic acid are both included in the editing cassette.

The target sequence is associated with a PAM, which is a short nucleotide sequence recognized by the gRNA/nuclease complex. The precise PAM sequence and length requirements for different nucleic acid-guided nucleases vary; however, PAMs typically are 2-7 base-pair sequences adjacent or in proximity to the target sequence and, depending on the nuclease, can be 5′ or 3′ to the target sequence. Engineering of the PAM-interacting domain of a nucleic acid-guided nuclease may allow for alteration of PAM specificity, improve target site recognition fidelity, decrease target site recognition fidelity, and increase the versatility of a nucleic acid-guided nuclease. In certain embodiments, the genome editing of a target sequence both introduces a desired DNA change to a target sequence, e.g., the genomic DNA of a cell, and removes, mutates, or renders inactive a proto-spacer (PAM) region in the target sequence; that is, the donor DNA often includes an alteration to the target sequence that prevents binding of the nuclease at the PAM in the target sequence after editing has taken place. Rendering the PAM at the target sequence inactive precludes additional editing of the cell genome at that target sequence, e.g., upon subsequent exposure to a nucleic acid-guided nuclease complexed with a synthetic guide nucleic acid in later rounds of editing. Thus, cells having the desired target sequence edit and an altered PAM can be selected using a nucleic acid-guided nuclease complexed with a synthetic guide nucleic acid complementary to the target sequence. Cells that did not undergo the first editing event will be cut rendering a double-stranded DNA break, and thus will not continue to be viable. The cells containing the desired target sequence edit and PAM alteration will not be cut, as these edited cells no longer contain the necessary PAM site and will continue to grow and propagate.

The range of target sequences that nucleic acid-guided nucleases can recognize is constrained by the need for a specific PAM to be located near the desired target sequence. As a result, it often can be difficult to target edits with the precision that is necessary for genome editing. It has been found that nucleases can recognize some PAMs very well (e.g., canonical PAMs), and other PAMs less well or poorly (e.g., non-canonical PAMs). Because the methods disclosed herein allow for identification of edited cells in a large background of unedited cells, the methods allow for identification of edited cells where the PAM is less than optimal; that is, the methods for identifying edited cells herein allow for identification of edited cells even if editing efficiency is very low. Additionally, the present methods expand the scope of target sequences that may be edited since edits are more readily identified, including cells where the genome edits are associated with less functional PAMs.

As for the nuclease component of the nucleic acid-guided nuclease editing system, the polynucleotide sequence encoding the nucleic acid-guided nuclease can be codon optimized for expression in particular cells, such as archaeal, prokaryotic or eukaryotic cells. Eukaryotic cells can be yeast, fungi, algae, plant, animal, or human cells. Eukaryotic cells may be those of or derived from a particular organism, such as a mammal, including but not limited to human, mouse, rat, rabbit, dog, or non-human mammal including non-human primate. The choice of nucleic acid-guided nuclease to be employed depends on many factors, such as what type of edit is to be made in the target sequence and whether an appropriate PAM is located close to the desired target sequence. Nucleases of use in the methods described herein include but are not limited to Cas 9, Cas 12/CpfI, MAD2, or MAD7 or other MADzymes. As with the guide nucleic acid, the nuclease may be encoded by a DNA sequence on a vector (e.g., the engine vector) and be under the control of a constitutive or an inducible promoter. Again, at least one of and preferably both of the nuclease and guide nucleic acid are under the control of an inducible promoter.

Another component of the nucleic acid-guided nuclease system is the donor nucleic acid. In some embodiments, the donor nucleic acid is on the same polynucleotide (e.g., vector or editing (CREATE) cassette) as the guide nucleic acid. The donor nucleic acid is designed to serve as a template for homologous recombination with a target sequence nicked or cleaved by the nucleic acid-guided nuclease as a part of the gRNA/nuclease complex. A donor nucleic acid polynucleotide may be of any suitable length, such as about or more than about 30, 35, 40, 45, 50, 75, 100, 150, 200, 500, 1000, 2500, 5000 nucleotides or more in length. In certain preferred aspects, the donor nucleic acid can be provided as an oligonucleotide of between 40-300 nucleotides, more preferably between 50-250 nucleotides. The donor nucleic acid comprises a region that is complementary to a portion of the target sequence (homology arm). When optimally aligned, the donor nucleic acid overlaps with (is complementary to) the target sequence by, e.g., about 10, 15, 20, 25, 30, 35, 40, 50, 60, 70, 80, 90 or more nucleotides. In many embodiments, the donor nucleic acid comprises two homology arms (regions complementary to the target sequence) flanking the mutation or difference between the donor nucleic acid and the target template. The donor nucleic acid comprises at least one mutation or alteration compared to the target sequence, such as an insertion, deletion, modification, or any combination thereof compared to the target sequence.

Often the donor nucleic acid is provided as an editing cassette, which is inserted into a vector backbone where the vector backbone may comprise a promoter driving transcription of the gRNA and the donor nucleic acid. Moreover, there may be more than one, e.g., two, three, four, or more guide nucleic acid/donor nucleic acid cassettes inserted into an engine vector, where the guide nucleic acids are under the control of separate, different promoters, separate, like promoters, or where all guide nucleic acid/donor nucleic acid pairs are under the control of a single promoter. (See, e.g., U.S. Ser. No. 16/275,465, filed 14 Feb. 2019, drawn to multiple CREATE cassettes.) The promoter driving transcription of the gRNA and the donor nucleic acid (or driving more than one gRNA/donor nucleic acid pair) is optionally an inducible promoter (and in bacterial systems is preferably an inducible promoter) and the promoter driving transcription of the nuclease is optionally an inducible promoter as well.

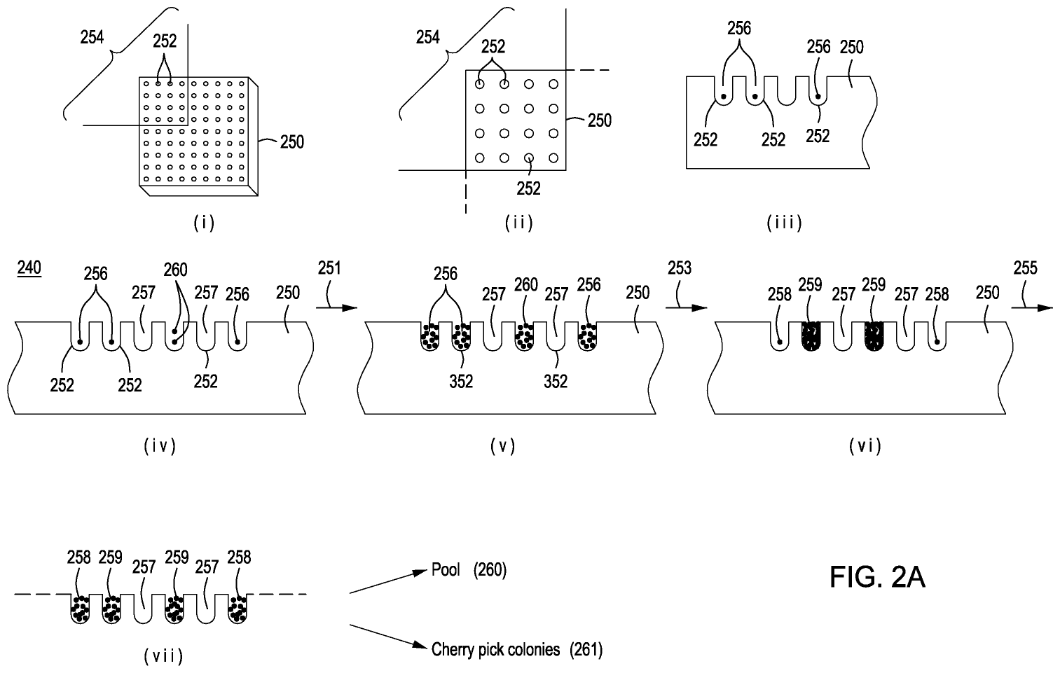

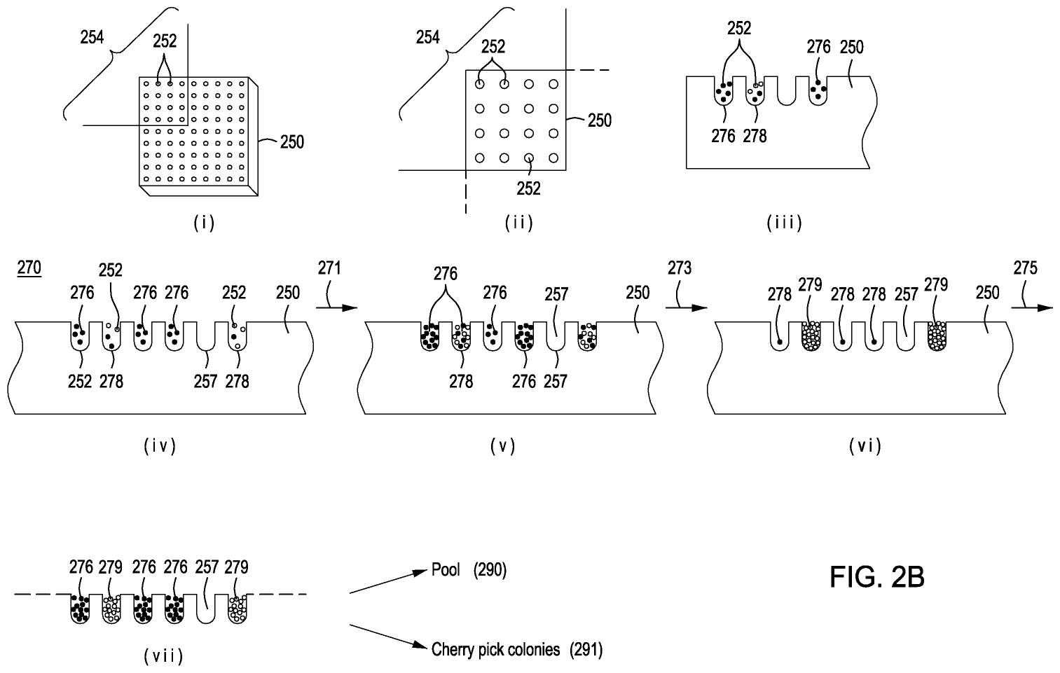

Inducible editing is advantageous in that substantially or largely isolated cells can be grown for several to many cell doublings before editing is initiated, which increases the likelihood that cells with edits will survive, as the double-strand cuts caused by active editing are largely toxic to the cells. This toxicity results both in cell death in the edited colonies, as well as a lag in growth for the edited cells that do survive but must repair and recover following editing. However, once the edited cells have a chance to recover, the size of the colonies of the edited cells will eventually catch up to the size of the colonies of unedited cells (e.g., the process of “normalization” or growing colonies to “terminal size”; see, e.g., FIGS. 2A and 2B described infra).

In addition to the donor nucleic acid, an editing cassette may comprise one or more primer sites. The primer sites can be used to amplify the editing cassette by using oligonucleotide primers; for example, if the primer sites flank one or more of the other components of the editing cassette.

Also, as described above, the donor nucleic acid may comprise—in addition to at least one mutation relative to a target sequence—one or more PAM sequence alterations that mutate, delete or render inactive the PAM site in the target sequence. The PAM sequence alteration in the target sequence renders the PAM site “immune” to the nucleic acid-guided nuclease and protects the target sequence from further editing in subsequent rounds of editing if the same nuclease is used.

The editing cassette also may comprise a barcode. A barcode is a unique DNA sequence that corresponds to the donor DNA sequence such that the barcode can identify the edit made to the corresponding target sequence. The barcode can comprise greater than four nucleotides. In some embodiments, the editing cassettes comprise a collection of donor nucleic acids representing, e.g., gene-wide or genome-wide libraries of donor nucleic acids. The library of editing cassettes is cloned into vector backbones where, e.g., each different donor nucleic acid design is associated with a different barcode, or, alternatively, each different cassette molecule is associate with a different barcode.

Additionally, in some embodiments, an expression vector or cassette encoding components of the nucleic acid-guided nuclease system further encodes a nucleic acid-guided nuclease comprising one or more nuclear localization sequences (NLSs), such as about or more than about 1, 2, 3, 4, 5, 6, 7, 8, 9, 10, or more NLSs. In some embodiments, the engineered nuclease comprises NLSs at or near the amino-terminus, NLSs at or near the carboxy-terminus, or a combination.

### Exemplary Workflows for Editing, Enrichment, and Selection of Edited Cells

The methods described herein provide phenotypic screening of edited cells via fluorescent screening reagents and imaging in a solid wall device in which the cells are grown. The combination of isolation or substantial isolation, initial cell growth, editing, fluorescent imaging and selection of desired cells allows for direct selection of edited colonies of cells in a single device. In art methods known in the past, a researcher may need to singulate a large number of cell colonies and perform many fluidically separate measurements in 96-well or 384-well plates. For a 1000-plex library, a researcher would need to plate, recover and assay well over 1000 cell colonies to sample>90% of the edits, given that some cell colonies will not be edited or will comprise redundant edits. Phenotypic screening of libraries in this manner is expensive and labor intensive. The methods and modules presented herein allow a researcher to singulate and edit cells on a solid wall device, as well as to perform phenotypic screening through, e.g., increased or decreased fluorescence using, e.g., a fluorescently-labeled antibody used to screen for a metabolite of interest.

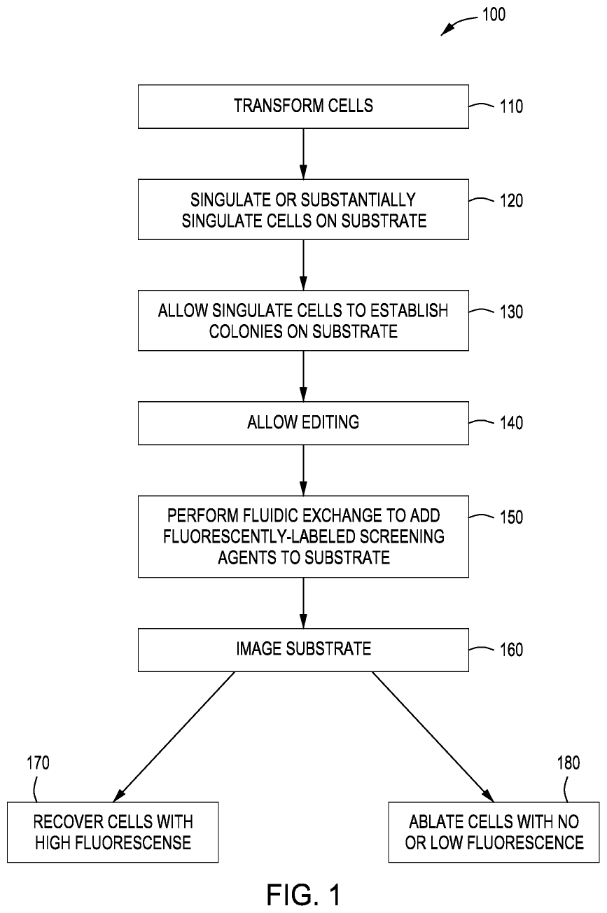

FIG. 1 shows simplified flow charts for an exemplary method 100 for transforming, isolating, editing, and imaging of edited cells. Looking at FIG. 1, method 100 begins by transforming cells 110 with the components necessary to perform nucleic acid-guided nuclease editing. For example, the cells may be transformed simultaneously with separate engine and editing vectors; the cells may already be transformed with an engine vector expressing the nuclease (e.g., the cells may have already been transformed with an engine vector or the coding sequence for the nuclease may be stably integrated into the cellular genome) such that only the editing vector needs to be transformed into the cells; or the cells may be transformed with a single vector comprising all components required to perform nucleic acid-guided nuclease genome editing.

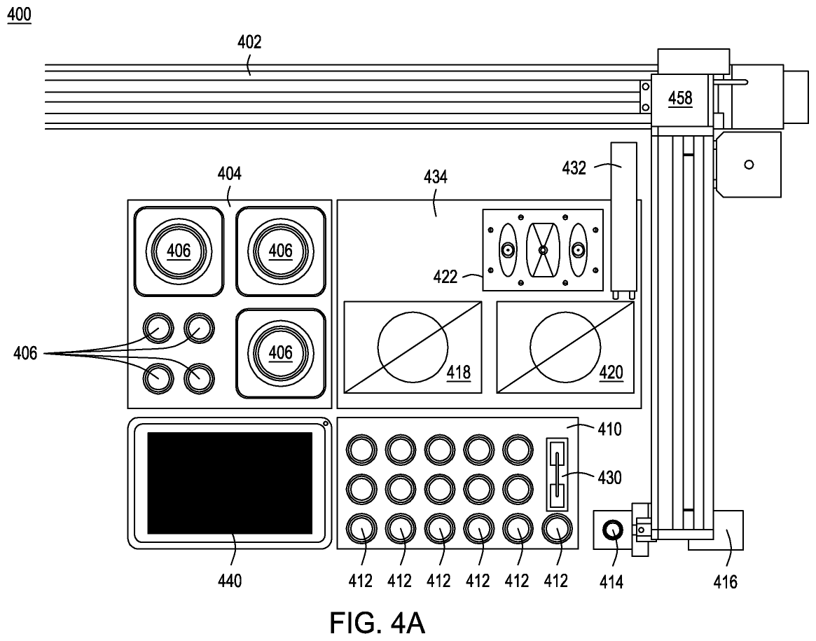

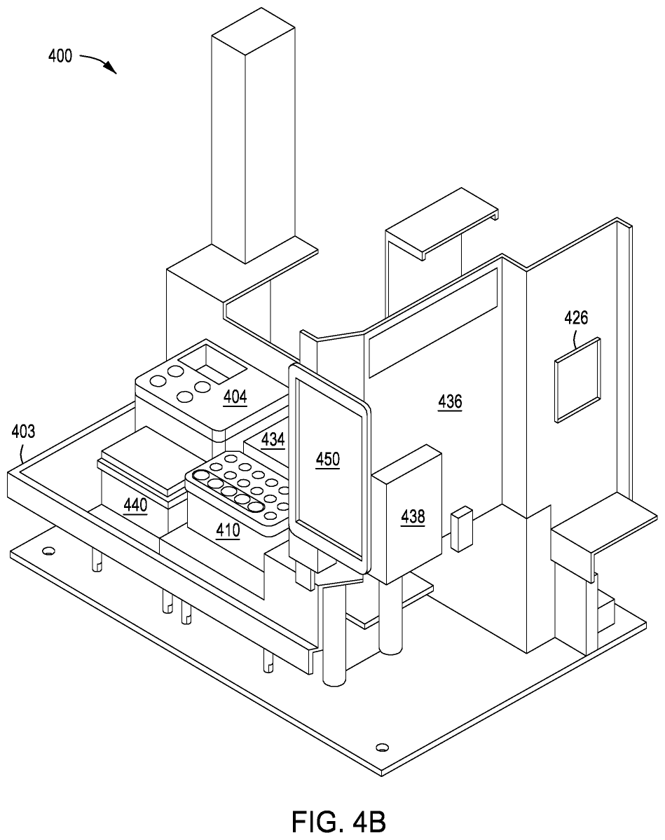

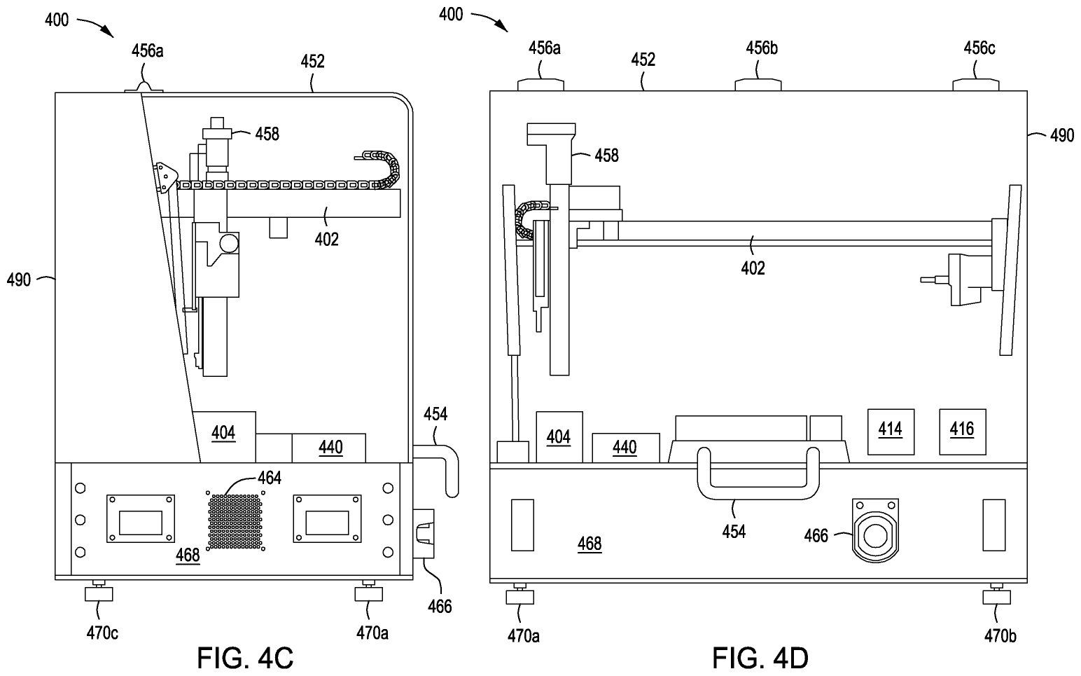

A variety of delivery systems can be used to introduce (e.g., transform or transfect) nucleic acid-guided nuclease editing system components into a host cell 110. These delivery systems include the use of yeast systems, lipofection systems, microinjection systems, biolistic systems, virosomes, liposomes, immunoliposomes, polycations, lipid:nucleic acid conjugates, virions, artificial virions, viral vectors, electroporation, cell permeable peptides, nanoparticles, nanowires, exosomes. Alternatively, molecular trojan horse liposomes may be used to deliver nucleic acid-guided nuclease components across the blood brain barrier. Of interest, particularly in the context of an automated multi-module cell editing instrument is the use of electroporation, particularly flow-through electroporation (either as a stand-alone instrument or as a module in an automated multi-module system) as described in, e.g., U.S. Ser. No. 16/147,120, filed 28 Sep. 2018; Ser. No. 16/147,353, filed 28 Sep. 2018; Ser. No. 16/147,865, filed 30 Sep. 2018; and Ser. No. 16/147,871, filed 30 Sep. 2018. If the solid wall isolation or substantial isolation/incubation/editing and normalization module is one module in an automated multi-module cell editing instrument as described herein infra in relation to FIGS. 4A-4D, the cells are likely transformed in an automated cell transformation module.

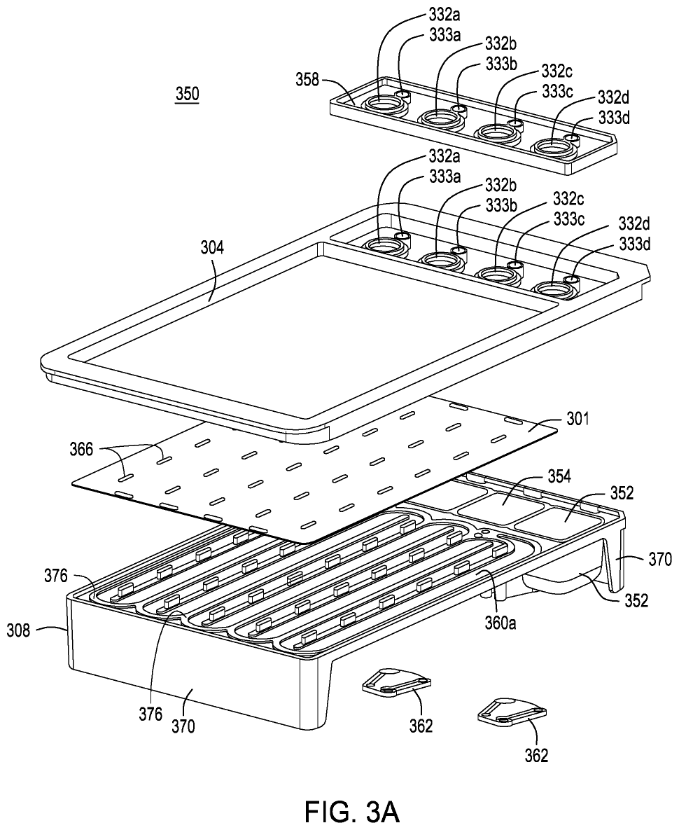

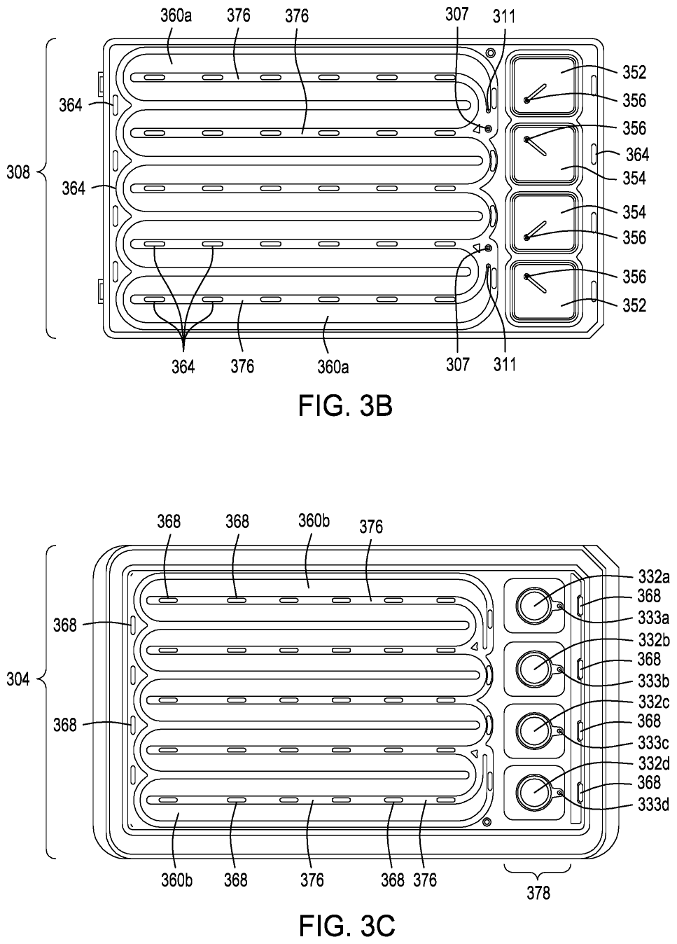

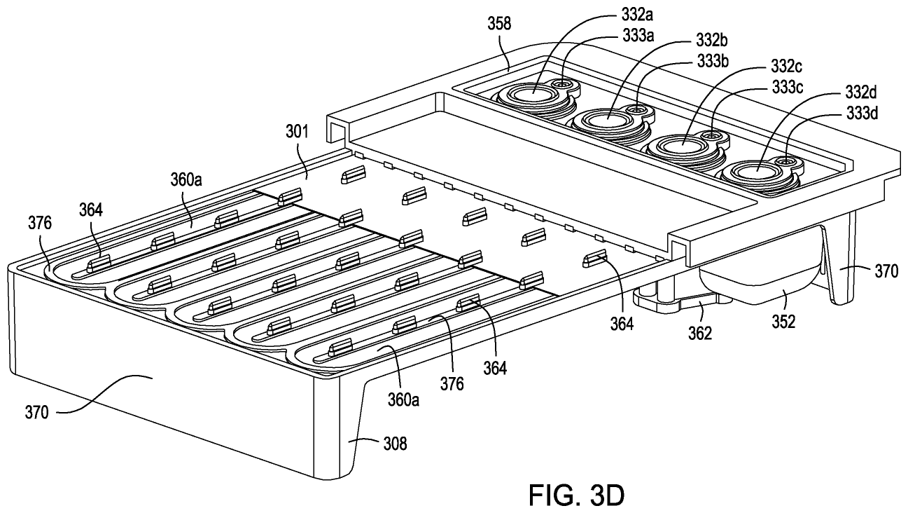

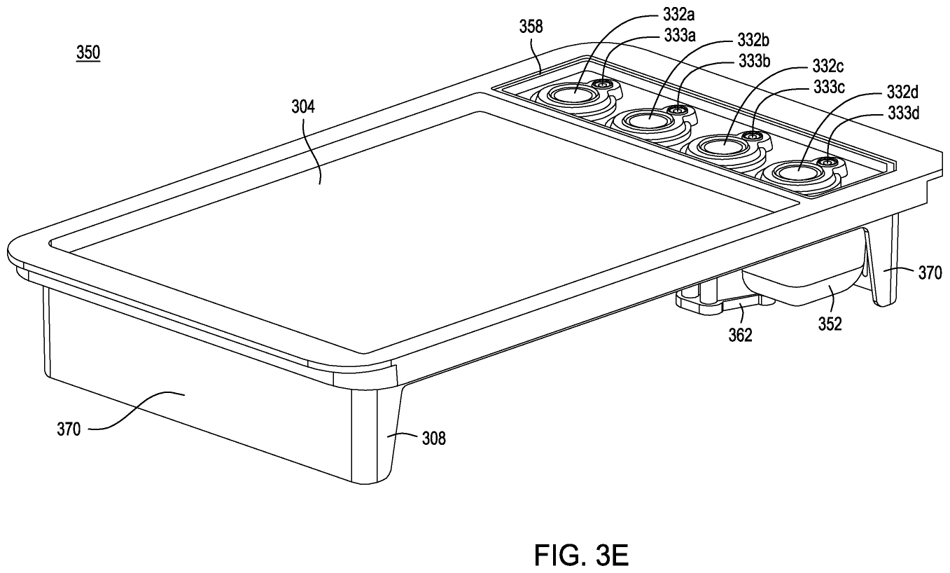

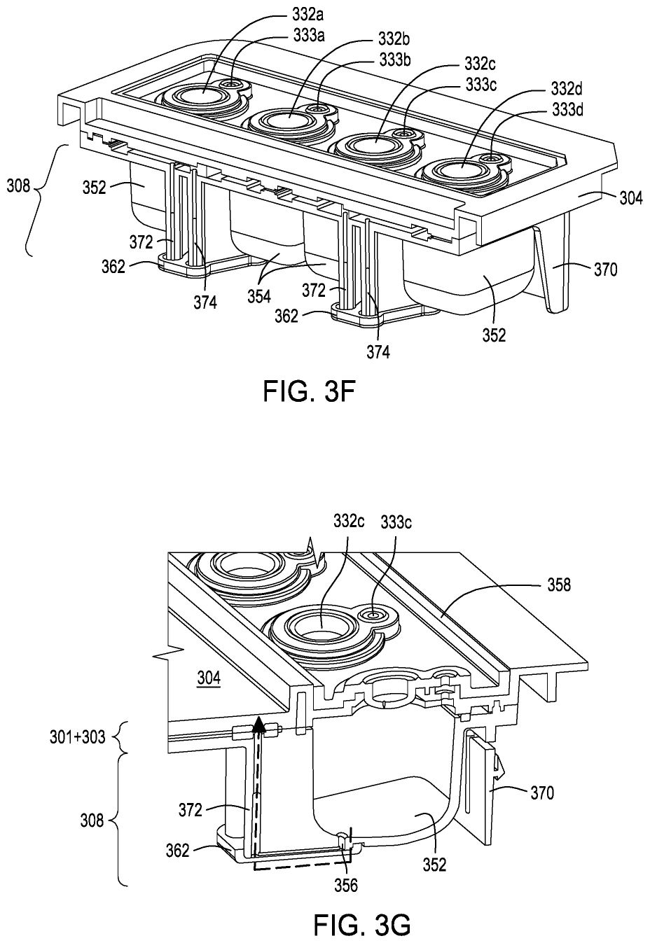

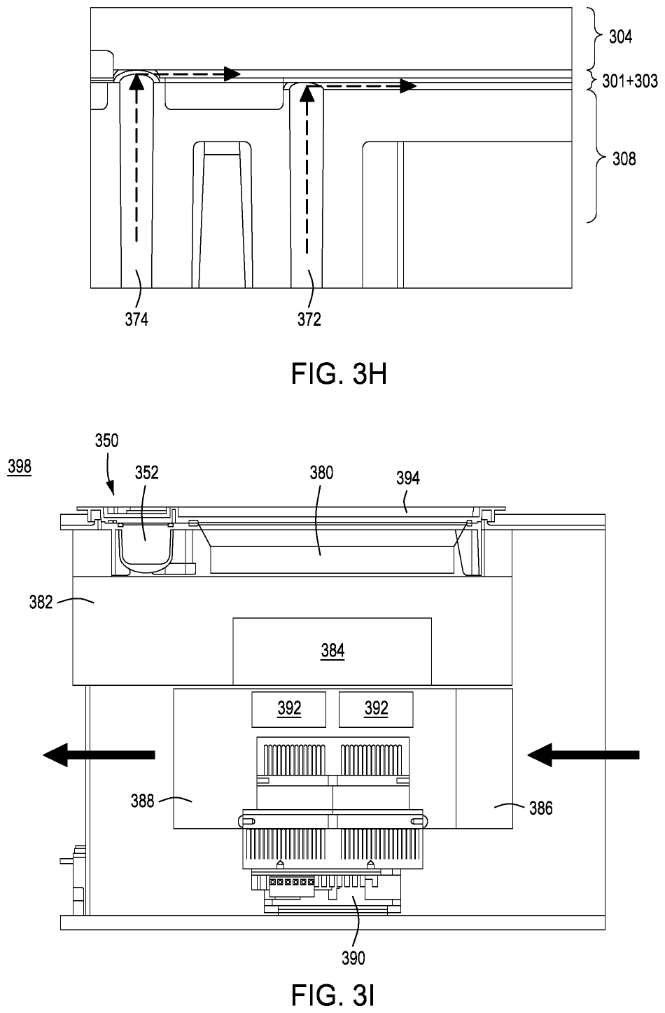

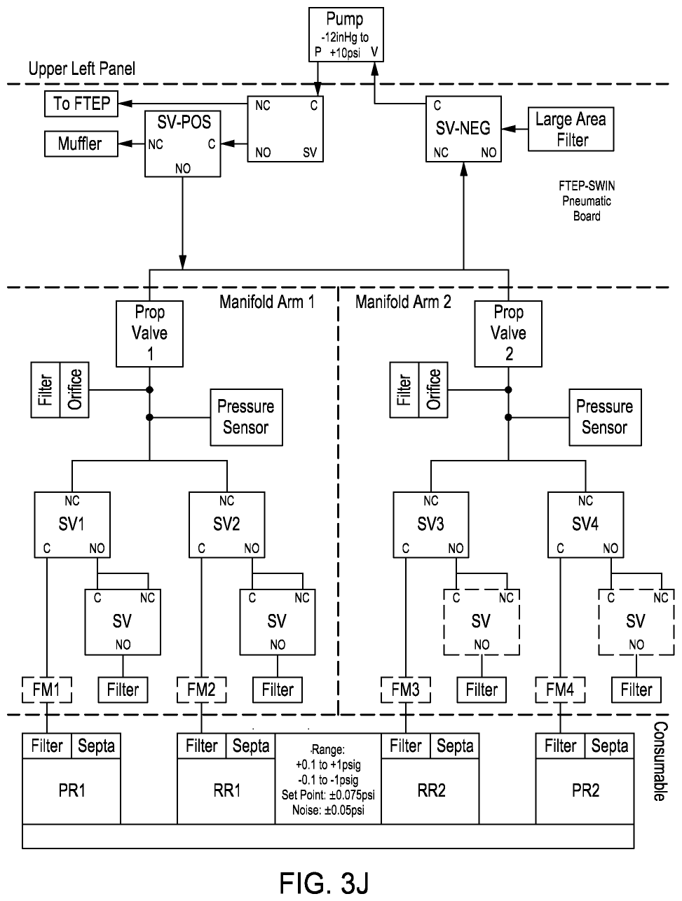

After the cells are transformed with the components necessary to perform nucleic acid-guided nuclease editing, the cells are substantially or largely isolated 120; that is, the cells are diluted (if necessary) in a liquid culture medium so that the cells, when delivered to a substrate for isolation, are substantially separated from one another and can form colonies that are substantially separated from one another. For example, if a solid wall device is used (described infra in relation to FIG. 3A-3N), the cells are diluted such that when delivered to the solid wall device the cells fill the microwells of the solid wall device in a Poisson or substantial Poisson distribution. In one example (illustrated in FIG. 2A), isolation is accomplished when an average of ½ cell is delivered to each microwell; that is, where some microwells contain one cell and other microwells contain no cells. Alternatively, a substantial Poisson distribution of cells occurs when two to several (2, 3, 4, 5, 6, 7, 8, 9, 10, 11, 12, 13, 14, 15, 16, 17, 18, 19, 20, 21, 22, 23, 24, 25, 26, 27, 28, 29, 30 or up to 50, but preferably 10 or less, or preferably 5 or less) cells are delivered to a microwell (illustrated in FIG. 2B).

Once the cells have been isolated or substantially or largely isolated 120, the cells preferably are allowed to grow to, e.g., between 2 and 130, or between 5 and 120, or between 10 and 100 doublings, or at least 2 doublings, or at least 5 doublings, or at least 10 or 20 doublings, establishing clonal colonies 130. After colonies are established, editing proceeds 140. In some systems an inducible system is used where at least the gRNA is and in some embodiments the nuclease is under the control of an inducible promoter. In some embodiments, the inducible promoter is a pL promoter; however, in other embodiments, different inducible promoters may be used to drive transcription of the nuclease and gRNA or in some embodiments only the gRNA is under control of an inducible promoter. For example, a number of gene regulation control systems have been developed for the controlled expression of genes in plant, microbe and animal cells, including mammalian cells. These systems include the tetracycline-controlled transcriptional activation system (Tet-On/Tet-Off, Clontech, Inc. (Palo Alto, Calif.); Bujard and Gossen, PNAS, 89(12):5547-5551 (1992)), the Lac Switch Inducible system (Wyborski et al., Environ Mol Mutagen, 28(4):447-58 (1996); DuCoeur et al., Strategies 5(3):70-72 (1992); and U.S. Pat. No. 4,833,080), the ecdysone-inducible gene expression system (No et al., PNAS, 93(8):3346-3351 (1996)), the cumate gene-switch system (Mullick et al., BMC Biotechnology, 6:43 (2006)), and the tamoxifen-inducible gene expression (Zhang et al., Nucleic Acids Research, 24:543-548 (1996)) as well as others. However, the pL promoter is a particularly useful inducible promoter because the pL promoter is activated by an increase in temperature, to, e.g., 42° C., and deactivated by returning the temperature to, e.g., 30° C. With other inducible systems that are activated by the presence or absence of a particular molecular compound, activating the inducible promoter requires addition of or removal of a molecular compound from the culture medium, thus requiring liquid handling, medium exchange, wash steps and the like.

Once editing is complete, a fluid exchange is performed to add a screening agent to the solid wall device to screen for a desired phenotype, and in some embodiments, the screening agent is fluorescently-labeled so that the screening agent can be easily imaged on the solid wall device. Optionally, before fluid exchange takes place a baseline fluorescence level may be measured. Fluorescence is a form of luminescence that results from matter emitting light of a certain wavelength after absorbing electromagnetic radiation. Molecules that re-emit light upon absorption of light are called fluorophores. The coupling of fluorescent moieties to antibodies to create labeled antibody reagents, first reported over 60 years ago, has become a routine and important procedure in the biological sciences. Often, a succinimidyl-ester functional group is attached to a fluorophore core and this functionality confers reaction specificity with primary amines to form fluorophore-antibody conjugates. Fluorescence imaging fluorescent dyes and fluorescent proteins allow one to experimentally observe the dynamics of gene expression, protein expression and molecular interactions in living cells, serving as a precise, quantitative tool. Rapid visualization of cell surface proteins or secreted proteins can be achieved through fluorescent antibody techniques that attach a fluorescent marker or fluorogen to the constant region of an antibody, resulting in a fluorescent screening or reporter molecule that is quick to use, easy to see and measure and is able to bind the target protein or molecule with high specificity. Fluorescent methods may be direct, in which a labeled monoclonal antibody binds an antigen; or indirect, in which secondary polyclonal antibodies bind antibodies that react to a prepared antigen.

Advantages to using fluorescent imaging include non-invasive imaging in vivo; probes may be designed to be extremely sensitive to detecting biological molecules like DNA, RNA, and proteins; multiple fluorophores can be detected within cell samples allowing for an easy way to integrate standards and a control; fluorescently labeled molecules used in imaging can be stored for months while other molecules—like ones that are radiolabeled—decay over a few days; most fluorophores can be safely and sufficiently handled with gloves, while for example, radioisotopes may require lead shields or other protection; and many fluorophores require minimal disposal methods while radioactive wastes require regulated disposal and long-term handling, which also aids in lowering the cost needed to utilize these products.

Once medium or fluid exchange has taken place, imaging is performed. As described in more detail infra, imaging of cell colonies growing in the wells of the solid wall device or SWIIN is desired in many implementations including for, e.g., monitoring both cell growth and device performance and for phenotypic screening of the edited cells. Fluorescence imaging in the SWIIN requires backlighting, condensation management and a system-level approach to temperature control, air flow, and thermal management. Since some wavelengths of fluorescence are beyond the range of the human eye, charged-coupled devices (CCD) are used to accurately detect light and image the emission, typically in the 300-800 nm range. One of the advantages of fluorescent imaging is that intensity of emitted light behaves substantially linearly in regard to the quantity of fluorescent molecules provided; however, this feature is obviously contingent that the absorbed light intensity and wavelength are constant. The actual image is usually in a 12-bit or 16-bit data format. The main components of fluorescence imaging systems are: 1) an excitation source, which typically is a device that produces either a broad-wavelength source like UV light, or a narrow wavelength source like a laser; 2) light display optics, the mechanism by which light illuminates the sample; 3) light assortment optics, which is the collection method of the light itself typically constituting lenses, mirrors, and filters; 4) filtration of emitted light, involving optical filters to ensure that reflected and scattered light are not included with the fluorescence; and 5) detection, amplification, and visualization with either photomultiplier (PMT) or charge-couple device (CCD) to detect and quantify emitted protons.

Once imaging has been performed, the imaging information may be used to cherry pick or select cells that fluoresce at a high (or, if desired, a low or no) level by recovering cells from specific wells 170, or by using UV light to irradiate or ablate specific wells 180 to eliminate cells that fluoresce at a low (or, if desired, a high) level.

### Exemplary Modules for Editing, Enrichment, and Selection of Edited Cells

The instruments, methods, and modules described herein enable enhanced observed editing efficiency of nucleic acid-guided nuclease editing methods as the result of isolation or substantial isolation, incubation, editing, and normalization. The combination of the isolation or substantial isolation, incubation, editing and normalization processes overcomes the growth bias in favor of unedited cells—and the fitness effects of editing, including differential editing rates—thus allowing all cells “equal billing” with one another. The result of the instruments, modules, and methods described herein is that even in nucleic acid-guided nuclease systems where editing is not optimal (such as in systems where non-canonical PAMs are targeted), there is an increase in the observed editing efficiency; that is, edited cells can be identified even in a large background of unedited cells. Observed editing efficiency can be improved up to 80% or more.

FIG. 2A depicts a solid wall device 250 and a workflow for isolating cells in microwells in the solid wall device, where in this exemplary workflow one or both of the gRNA and nuclease may optionally be under the control of an inducible promoter. At the top left of the figure (i), there is depicted solid wall device 250 with microwells 252. A section 254 of substrate 250 is shown at (ii), also depicting microwells 252. At (iii), a side cross-section of solid wall device 250 is shown, and microwells 252 have been loaded, where, in this embodiment, Poisson or substantial Poisson loading has taken place; that is, each microwell has one or no cells, and the likelihood that any one microwell has more than one cell is low. Note wells 256 have one cell loaded. At (iv), workflow 240 is illustrated where substrate 250 having microwells 252 shows microwells 256 with one cell per microwell, microwells 257 with no cells in the microwells, and one microwell 360 with two cells in the microwell. In step 251, the cells in the microwells are allowed to double approximately 2-150 times to form clonal colonies (v), then editing optionally is induced 253 by heating the substrate (e.g., for temperature-induced editing) or flowing chemicals under or over the substrate (e.g., sugars, antibiotics for chemical-induced editing) or by moving the solid wall device to a different medium, particularly facile if the solid wall device is placed on a membrane which forms the bottom of microwells 252 (membrane not shown).

After optional induction of editing 253, many cells in the colonies of cells that have been edited die as a result of the double-strand cuts caused by active editing and there is a lag in growth for the edited cells that do survive but must repair and recover following editing (microwells 258), where cells that do not undergo editing thrive (microwells 259) (vi). All cells are allowed to continue grow to establish colonies and normalize, where the colonies of edited cells in microwells 258 catch up in size and/or cell number with the cells in microwells 259 that do not undergo editing (vii). Once the cell colonies are normalized, either pooling 260 of all cells in the microwells can take place, in which case the cells are enriched for edited cells by eliminating the bias from non-editing cells and fitness effects from editing; alternatively, colony growth in the microwells is monitored after editing, and slow growing colonies (e.g., the cells in microwells 258) are identified and selected 261 (e.g., “cherry picked”) resulting in even greater enrichment of edited cells.

In growing the cells, the medium used will depend, of course, on the type of cells being edited—e.g., bacterial, yeast or mammalian. For example, medium for bacterial growth includes LB, SOC, M9 Minimal medium, and Magic medium; medium for yeast cell growth includes TPD, YPG, YPAD, and synthetic minimal medium; and medium for mammalian cell growth includes MEM, DMEM, IMDM, RPMI, and Hanks. For culture of adherent cells, cells may be disposed on beads or another type of scaffold suspended in medium. Most normal mammalian tissue-derived cells—except those derived from the hematopoietic system—are anchorage dependent and need a surface or cell culture support for normal proliferation. In the rotating growth vial described herein, microcarrier technology is leveraged. Microcarriers of particular use typically have a diameter of 100-300 μm and have a density slightly greater than that of the culture medium (thus facilitating an easy separation of cells and medium for, e.g., medium exchange) yet the density must also be sufficiently low to allow complete suspension of the carriers at a minimum stirring rate in order to avoid hydrodynamic damage to the cells. Many different types of microcarriers are available, and different microcarriers are optimized for different types of cells. There are positively charged carriers, such as Cytodex 1 (dextran-based, GE Healthcare), DE-52 (cellulose-based, Sigma-Aldrich Labware), DE-53 (cellulose-based, Sigma-Aldrich Labware), HLX 11-170 (polystyrene-based); collagen or ECM (extracellular matrix)-coated carriers, such as Cytodex 3 (dextran-based, GE Healthcare) or HyQ-sphere Pro-F 102-4 (polystyrene-based, Thermo Scientific); non-charged carriers, like HyQspheres P 102-4 (Thermo Scientific); or macroporous carriers based on gelatin (Cultisphere, Percell Biolytica) or cellulose (Cytopore, GE Healthcare).

The solid wall devices can provide populations of cells with varying edits and/or percentages of clonality. It has been determined that flowing medium over the retentate side surface of the SWIIN, e.g., a tangential or sheer flow across the top of the perforate member of the SWIIN, will flush off the tops (“muffin tops”) of the cell colonies over growing the wells of the SWIIN without contaminating the wells containing other cells; i.e., depositing flushed cells in wells. In one embodiment, cells are allowed to grow to a desired state, for example when some of the colonies—fast-growing colonies, which are likely to be unedited cells—have over-grown the wells, then the “muffin tops” are flushed off. In a next round, the cells again are allowed to grow again to a desired state, for example when some or many more of the colonies have over-grown the wells, then the “muffin tops” are flushed off, and additional rounds of cell growth and flushing/collection can continue as desired. After a desired number of rounds of cell growth and collection, 1) the cells can be collected and pooled, 2) certain collections may be discarded (such as the first collection of cells which are more likely to be unedited cells) and the rest of the collections pooled, or 3) each round of collection may be kept separate and analyzed for clonality, percentage of edited cells, etc. This embodiment requires monitoring of cell growth by, e.g., imaging, as described in relation to FIG. 3I.

FIG. 2B depicts a solid wall device 250 and a workflow for substantially isolating cells in microwells in a solid wall device, where in this workflow—as in the workflow depicted in FIG. 2A—optionally one or both of the gRNA and nuclease is under the control of an inducible promoter. At the top left of the figure (i), there is depicted a solid wall device 250 with microwells 252. A section 254 of substrate 250 is shown at (ii), also depicting microwells 252. At (iii), a side cross-section of solid wall device 250 is shown, and microwells 252 have been loaded, where, in this embodiment, substantial Poisson loading has taken place; that is, one microwell 257 has no cells, and some microwells 276, 278 have a few cells. In FIG. 2B, cells with active gRNAs are shown as solid circles, and cells with inactive gRNAs are shown as open circles. At (iv) workflow 270 is illustrated where substrate 250 having microwells 252 shows three microwells 276 with several cells all with active gRNAs, microwell 257 with no cells, and two microwells 278 with some cells having active gRNAs and some cells having inactive gRNAs. In step 371, the cells in the microwells are allowed to double approximately 2-150 times to form clonal colonies (v), then editing optionally is induced 273 by heating the substrate (e.g., for temperature-induced editing) or flowing chemicals under or over the substrate (e.g., sugars, antibiotics for chemical-induced editing) or by moving the solid wall device to a different medium, particularly facile if the solid wall device is placed on a membrane which forms the bottom of microwells 252.

After editing 273, many cells in the colonies of cells that have been edited die as a result of the double-strand cuts caused by active editing and there is a lag in growth for the edited cells that do survive but must repair and recover following editing (microwells 278), where cells that do not undergo editing thrive (microwells 279) (vi). Thus, in microwells 278 where only cells with active gRNAs reside (cells depicted by solid circles), most cells die off; however, in microwells 279 containing cells with inactive gRNAs (cells depicted by open circles), cells continue to grow and are not impacted by active editing. The cells in each microwell (278 and 279) are allowed to grow to continue to establish colonies and normalize, where the colonies of edited cells in microwells 278 catch up in size and/or cell number with the unedited cells in microwells 279 that do not undergo editing (vii). Note that in this workflow 270, the colonies of cells in the microwells are not clonal; that is, not all cells in a well arise from a single cell. Instead, the cell colonies in the well may be mixed colonies, arising in many wells from two to several different cells. Once the cell colonies are normalized, either pooling 290 of all cells in the microwells can take place, in which case the cells are enriched for edited cells by eliminating the bias from non-editing cells and fitness effects from editing or cells may be flushed from the SWIIN and collected at various time points; alternatively, colony growth in the microwells is monitored after editing, and slow growing colonies (e.g., the cells in microwells 278) are identified and selected 291 (e.g., “cherry picked”) resulting in even greater enrichment of edited cells.

FIGS. 3A through 3I depict various components of different embodiments and components of a solid wall isolation or substantial isolation, incubation, editing and either normalization or cherry picking module (“solid wall isolation/incubation/normalization module” or “SWIIN”) suitable for isolating (or substantially isolating) cells of all types, growing cells for an initial, e.g., 2-150 rounds of cell division, optionally inducing editing, and either normalizing or cherry picking the resulting cell colonies. The SWIIN modules presented may be stand-alone devices, or, often, one module in an automated multi-module cell processing instrument.

FIG. 3A depicts an embodiment of a SWIIN module 350 from an exploded top perspective view. The SWIIN module embodiment described in relation to FIGS. 3A-3I provides advantages, such as the positioning of the reservoirs and reservoir ports below the retentate and permeate serpentine channels to minimize instantaneous flow of fluid in the reservoirs through the reservoir ports and into channels that connect the reservoir ports to the retentate and permeate channels. Instead, flow is controlled by the application of pressure (positive or negative) and an appropriate time chosen by the user. Also, in SWIIN module 350 the retentate member is formed on the bottom of a top of a SWIIN module component and the permeate member is formed on the top of the bottom of a SWIIN module component Eliminating a “singulation assembly” and SWIIN cover, vastly simplifies manufacture and assembly of the SWIIN module and decreases costs. In addition, a SWIIN assembly comprising the SWIIN module 350 comprises features to manage condensation, which allows for improved imaging of the wells. These features are described infra. For alternative and more detailed descriptions of a SWIIN, see U.S. Ser. No. 16/399,988, filed 30 Apr. 2019; Ser. No. 16/454,865, filed 26 Jun. 2019; and Ser. No. 16/540,606, filed 14 Aug. 2019.

SWIIN module 350 in FIG. 3A comprises from the top down, a reservoir gasket or cover 358, a retentate member 304 (where a retentate flow channel cannot be seen in this FIG. 3A), a perforated member 301 swaged with a filter (filter not seen in FIG. 3A), a permeate member 308 comprising integrated reservoirs (permeate reservoirs 352 and retentate reservoirs 354), and two reservoir seals 362, which seal the bottom of permeate reservoirs 352 and retentate reservoirs 354. A permeate channel 360a can be seen disposed on the top of permeate member 308, defined by a raised portion 376 of serpentine channel 360a, and ultrasonic tabs 364 can be seen disposed on the top of permeate member 308 as well. The perforations that form the wells on perforated member 301 are not seen in this FIG. 3A; however, through-holes 366 to accommodate the ultrasonic tabs 364 are seen. In addition, supports 370 are disposed at either end of SWIIN module 350 to support SWIIN module 350 and to elevate permeate member 408 and retentate member 404 above reservoirs 352 and 354 to minimize bubbles or air entering the fluid path from the permeate reservoir to serpentine channel 360a or the fluid path from the retentate reservoir to serpentine channel 360b (neither fluid path is seen in this FIG. 3A, but see FIG. 3J).

In this FIG. 3A, it can be seen that the serpentine channel 360a that is disposed on the top of permeate member 308 traverses permeate member 308 for most of the length of permeate member 308 except for the portion of permeate member 308 that comprises permeate reservoirs 352 and retentate reservoirs 354 and for most of the width of permeate member 308. As used herein with respect to the distribution channels in the retentate member or permeate member, “most of the length” means about 95% of the length of the retentate member or permeate member, or about 90%, 85%, 80%, 75%, or 70% of the length of the retentate member or permeate member. As used herein with respect to the distribution channels in the retentate member or permeate member, “most of the width” means about 95% of the width of the retentate member or permeate member, or about 90%, 85%, 80%, 75%, or 70% of the width of the retentate member or permeate member.

In this embodiment of a SWIIN module, the perforated member includes through-holes to accommodate ultrasonic tabs disposed on the permeate member. Thus, in this embodiment the perforated member is fabricated from 316 stainless steel, and the perforations form the walls of microwells while a filter or membrane is used to form the bottom of the microwells. Typically, the perforations (microwells) are approximately 150 μm-200 μm in diameter, and the perforated member is approximately 125 μm deep, resulting in microwells having a volume of approximately 2.5 nl, with a total of approximately 200,000 microwells. The distance between the microwells is approximately 279 μm center-to-center. Though here the microwells have a volume of approximately 2.5 nl, the volume of the microwells may be from 1 to 25 nl, or preferably from 2 to 10 nl, and even more preferably from 2 to 4 nl. Generally, the number of cells loaded into a singulation device or singulation assembly ranges from between approximately 0.1× to 2.5× the number of perforations or microwells, or from between approximately 0.3× to 2.0× the number of perforations or microwells, or from between approximately 0.5× to 1.5× the number of perforations or microwells. In general, the larger the plexity (e.g., complexity) of the library being used to edit a population of cells, a larger number of microwells or partitions is preferred. If, for example, a 10,000-plex library is used to edit a population of cells, a perforated member with 200,000 microwells or partitions would be more than adequate; however, if a 50,000-plex library is used to edit a population of cells, a perforated member (or two or more members in a compound SWIIN) with a total of 400,000 or more microwells or partitions may be preferred. The number of microwells in a single SWIIN module (e.g., not a compound SWIIN) may range from 20,000 to 500,000 microwells, or from 30,000 to 450,000 microwells, or from 50,000 to 400,000 microwells or from 100,000 to 300,000 microwells. For an even greater range of well number, compound SWIIN devices may be employed.

As for the filter or membrane, like the filter described previously, filters appropriate for use are solvent resistant, contamination free during filtration, and are able to retain the types and sizes of cells of interest. For example, in order to retain small cell types such as bacterial cells, pore sizes can be as low as 0.10 μm, however for other cell types (e.g., such as for mammalian cells), the pore sizes can be as high as 10.0 μm-20.0 μm or more. Indeed, the pore sizes useful in the cell concentration device/module include filters with sizes from 0.10 μm, 0.11 μm, 0.12 μm, 0.13 μm, 0.14 μm, 0.15 μm, 0.16 μm, 0.17 μm, 0.18 μm, 0.19 μm, 0.20 μm, 0.21 μm, 0.22 μm, 0.23 μm, 0.24 μm, 0.25 μm, 0.26 μm, 0.27 μm, 0.28 μm, 0.29 μm, 0.30 μm, 0.31 μm, 0.32 μm, 0.33 μm, 0.34 μm, 0.35 μm, 0.36 μm, 0.37 μm, 0.38 μm, 0.39 μm, 0.40 μm, 0.41 μm, 0.42 μm, 0.43 μm, 0.44 μm, 0.45 μm, 0.46 μm, 0.47 μm, 0.48 μm, 0.49 μm, 0.50 μm and larger. The filters may be fabricated from any suitable material including cellulose mixed ester (cellulose nitrate and acetate) (CME), polycarbonate (PC), polyvinylidene fluoride (PVDF), polyethersulfone (PES), polytetrafluoroethylene (PTFE), nylon, or glass fiber. The perforated member and filter are swaged together; that is, the perforated member and filter are pressed together under high pressure (e.g., 20 kpsi). In some embodiments, the holes or partitions in the perforated member are etched with a slight taper and the filter material is pressed into the opening then relaxed to form an effective swage, allowing for an adhesive-free coupling. As an alternative, an adhesive could be employed.

FIG. 3B is a top-down view of permeate member 308, showing serpentine channel 360a (the portion of the serpentine channel disposed in permeate member 308) defined by raised portion 376 of serpentine channel 360a, permeate reservoirs 352, retentate reservoirs 354, reservoir ports 356 (two of the four of which are labeled), ultrasonic tabs 364 disposed at each end of permeate member 308 and on the raised portion 376 of serpentine channel 360a of permeate member 308, two permeate ports 311, and two retentate ports 307 are also seen.

FIG. 3C is a bottom-up view of retentate member 304, showing serpentine channel 360b (the portion of the serpentine channel disposed in retentate member 308) defined by the raised portion 376 of the serpentine channel 360b. Also seen is an integrated reservoir cover 378 for the permeate and retentate reservoirs that mate with permeate reservoirs 352 and retentate reservoirs 354 on the permeate member. The integrated reservoir cover 378 comprises reservoir access apertures 332a, 332b, 332c, and 332d, as well as pneumatic ports 333a, 333b, 333c and 333d. As with previous embodiments, the serpentine channel 360a of permeate member 308 and the serpentine channel 360b of retentate member 304 mate to form the top (retentate member) and bottom (permeate member) of a mated serpentine channel. The footprint length of the serpentine channel structure is from, e.g., from 80 mm to 500 mm, from 100 mm to 400 mm, or from 150 mm to 250 mm. In some aspects, the entire footprint width of the channel structure is from 50 mm to 200 mm, from 75 mm to 175 mm, or from 100 mm to 150 mm.

The cross-section configuration of the mated serpentine channel may be round, elliptical, oval, square, rectangular, trapezoidal, or irregular. If square, rectangular, or another shape with generally straight sides, the cross section may be from about 2 mm to 15 mm wide, or from 3 mm to 12 mm wide, or from 5 mm to 10 mm wide. If the cross section of the mated serpentine channel is generally round, oval or elliptical, the radius of the channel may be from about 3 mm to 20 mm in hydraulic radius, or from 5 mm to 15 mm in hydraulic radius, or from 8 mm to 12 mm in hydraulic radius.

As in previous embodiments, disposed between serpentine channels 360a and 360b is perforated member 301 (adjacent retentate member 304) and filter 303 (adjacent permeate member 308), where filter 303 is swaged with perforated member 301. Serpentine channels 360a and 360b can have approximately the same volume or a different volume. For example, each “side” or portion 360a, 360b of the serpentine channel may have a volume of, e.g., 2 mL, or serpentine channel 360a of permeate member 308 may have a volume of 2 mL, and the serpentine channel 360b of retentate member 304 may have a volume of, e.g., 3 mL. The volume of fluid in the serpentine channel may range from about 2 mL to about 80 mL, or about 4 mL to 60 mL, or from 5 mL to 40 mL, or from 6 mL to 20 mL (note these volumes apply to a SWIIN module comprising a, e.g., 50-500K perforation member). The volume of the reservoirs may range from 5 mL to 50 mL, or from 7 mL to 40 mL, or from 8 mL to 30 mL or from 10 mL to 20 mL, and the volumes of all reservoirs may be the same or the volumes of the reservoirs may differ (e.g., the volume of the permeate reservoirs is greater than that of the retentate reservoirs).

The serpentine channel portions 360a and 360b of the permeate member 308 and retentate member 304, respectively, are approximately 200 mm long, 130 mm wide, and 4 mm thick, though in other embodiments, the retentate and permeate members can be from 75 mm to 400 mm in length, or from 100 mm to 300 mm in length, or from 150 mm to 250 mm in length; from 50 mm to 250 mm in width, or from 75 mm to 200 mm in width, or from 100 mm to 150 mm in width; and from 2 mm to 15 mm in thickness, or from 4 mm to 10 mm in thickness, or from 5 mm to 8 mm in thickness. The retentate (and permeate) members may be fabricated from PMMA (poly(methyl methacrylate)) or other materials may be used, including polycarbonate, cyclic olefin co-polymer (COC), glass, polyvinyl chloride, polyethylene, polyamide, polypropylene, polysulfone, polyurethane, and co-polymers of these and other polymers. Preferably at least the retentate member is fabricated from a transparent material so that the cells can be visualized (see, e.g., FIG. 3I and the description thereof). For example, a video camera may be used to monitor cell growth by, e.g., density change measurements based on an image of an empty well, with phase contrast, or if, e.g., a chromogenic marker, such as a chromogenic protein, is used to add a distinguishable color to the cells. Chromogenic markers such as blitzen blue, dreidel teal, virginia violet, vixen purple, prancer purple, tinsel purple, maccabee purple, donner magenta, cupid pink, seraphina pink, scrooge orange, and leor orange (the Chromogenic Protein Paintbox, all available from ATUM (Newark, Calif.)) obviate the need to use fluorescence, although fluorescent cell markers, fluorescent proteins, and chemiluminescent cell markers may also be used.

Because the retentate member preferably is transparent, colony growth in the SWIIN module can be monitored by automated devices such as those sold by JoVE (ScanLag™ system, Cambridge, Mass.) (also see Levin-Reisman, et al., Nature Methods, 7:737-39 (2010)). Cell growth for, e.g., mammalian cells may be monitored by, e.g., the growth monitor sold by IncuCyte (Ann Arbor, Mich.) (see also, Choudhry, PLos One, 11(2):e0148469 (2016)). Further, automated colony pickers may be employed, such as those sold by, e.g., TECAN (Pickolo™ system, Mannedorf, Switzerland); Hudson Inc. (RapidPick™, Springfield, N.J.); Molecular Devices (QPix 400™ system, San Jose, Calif.); and Singer Instruments (PIXL™ system, Somerset, UK).

Due to the heating and cooling of the SWIIN module, condensation may accumulate on the retentate member which may interfere with accurate visualization of the growing cell colonies. Condensation of the SWIIN module 350 may be controlled by, e.g., moving heated air over the top of (e.g., retentate member) of the SWIIN module 350, or by applying a transparent heated lid over at least the serpentine channel portion 360b of the retentate member 304. See, e.g., FIG. 3I and the description thereof infra.

As with the embodiments described previously, in SWIIN module 350 cells and medium—at a dilution appropriate for Poisson or substantial Poisson distribution of the cells in the microwells of the perforated member—are flowed into serpentine channel 360b from ports in retentate member 304, and the cells settle in the microwells while the medium passes through the filter into serpentine channel 360a in permeate member 308. The cells are retained in the microwells of perforated member 301 as the cells cannot travel through filter 303. Appropriate medium may be introduced into permeate member 308 through permeate ports 311. The medium flows upward through filter 303 to nourish the cells in the microwells (perforations) of perforated member 301. Additionally, buffer exchange can be effected by cycling medium through the retentate and permeate members. In operation, the cells are deposited into the microwells, are grown for an initial, e.g., 2-100 doublings, editing is induced by, e.g., raising the temperature of the SWIIN to 42° C. to induce a temperature inducible promoter or by removing growth medium from the permeate member and replacing the growth medium with a medium comprising a chemical component that induces an inducible promoter.

Once editing has taken place, the temperature of the SWIIN may be decreased, or the inducing medium may be removed and replaced with fresh medium lacking the chemical component thereby de-activating the inducible promoter. The cells then continue to grow in the SWIIN module 350 until the growth of the cell colonies in the microwells is normalized. For the normalization protocol, once the colonies are normalized, the colonies are flushed from the microwells by applying fluid or air pressure (or both) to the permeate member serpentine channel 360a and thus to filter 303 and pooled. Alternatively, if cherry picking is desired, the growth of the cell colonies in the microwells is monitored, and slow-growing colonies are directly selected; or, fast-growing colonies are eliminated.

FIG. 3D is a top perspective view of a SWIIN module with the retentate and perforated members in partial cross section. In this FIG. 3D, it can be seen that serpentine channel 360a is disposed on the top of permeate member 308 is defined by raised portions 376 and traverses permeate member 308 for most of the length and width of permeate member 308 except for the portion of permeate member 308 that comprises the permeate and retentate reservoirs (note only one retentate reservoir 352 can be seen). Moving from left to right, reservoir gasket 358 is disposed upon the integrated reservoir cover 378 (cover not seen in this FIG. 3D) of retentate member 304. Gasket 358 comprises reservoir access apertures 332a, 332b, 332c, and 332d, as well as pneumatic ports 333a, 333b, 333c and 333d. Also at the far left end is support 370. Disposed under permeate reservoir 352 can be seen one of two reservoir seals 362. In addition to the retentate member being in cross section, the perforated member 301 and filter 303 (filter 303 is not seen in this FIG. 3D) are in cross section. Note that there are a number of ultrasonic tabs 364 disposed at the right end of SWIIN module 350 and on raised portion 376 which defines the channel turns of serpentine channel 360a, including ultrasonic tabs 364 extending through through-holes 366 of perforated member 301. There is also a support 370 at the end distal reservoirs 352, 354 of permeate member 308.

FIG. 3E is a side perspective view of an assembled SWIIIN module 350, including, from right to left, reservoir gasket 358 disposed upon integrated reservoir cover 378 (not seen) of retentate member 304. Gasket 358 may be fabricated from rubber, silicone, nitrile rubber, polytetrafluoroethylene, a plastic polymer such as polychlorotrifluoroethylene, or other flexible, compressible material. Gasket 358 comprises reservoir access apertures 332a, 332b, 332c, and 332d, as well as pneumatic ports 333a, 333b, 333c and 333d. Also at the far-left end is support 370 of permeate member 308. In addition, permeate reservoir 352 can be seen, as well as one reservoir seal 362. At the far-right end is a second support 370.

FIG. 3F is a side perspective view of the reservoir portion of permeate member 308 and retentate member 304, including gasket 358. Seen are permeate reservoirs 352 as the outside reservoirs, and retentate reservoirs 354 between permeate reservoirs 352. It should be apparent to one of ordinary skill in the art given the present description, however, that this particular configuration of reservoirs may be changed with permeate 352 and retentate 354 reservoirs alternating in position; with both permeate reservoirs 352 on one side of SWIIN module 350 and both retentate reservoirs 354 on the other side of SWIIN module 350, or the retentate reservoirs 354 may be positioned at the two sides with the permeate reservoirs 352 between the retentate reservoirs. Again, gasket 358 comprises reservoir access apertures 332a, 332b, 332c, and 332d, as well as pneumatic ports 333a, 333b, 333c and 333d. In addition, two reservoir seals 362 can be seen, each sealing one permeate reservoir 352 and one retentate reservoir 354. Also seen is support 370 at the “reservoir end” of permeate member 308.

FIG. 3G is a side perspective cross sectional view of permeate reservoir 352 of permeate member 308 and retentate member 304 and gasket 358. Reservoir access aperture 332c and pneumatic aperture 333c can be seen, as well as support 370. Also seen is perforated member 301 and filter 303 (filter 303 is not seen clearly in this FIG. 3G but is sandwiched in between perforated member 301 and permeate member 308). A fluid path 372 from permeate reservoir 352 to serpentine channel 360a in permeate member 308 can be seen, as can reservoir seal 362.

FIG. 3H is a small segment of a cross section of SWIIN module 350, showing the retentate member 304, perforated member 301, filter 303, and retentate member 308. FIG. 3H also shows a fluid path 372 from a permeate reservoir to the serpentine channel 360a disposed in permeate member 308, and a fluid path 374 from a retentate reservoir to the serpentine channel 360b disposed in permeate member 304. As mentioned previously, the reservoir architecture of this embodiment is particularly advantageous as bubbling is minimized. That is, because the reservoirs and reservoir ports are positioned below the retentate and permeate serpentine channels, there is no instantaneous flow of fluid in the reservoirs into channels that connect the reservoir ports to the retentate and permeate channels. Instead, flow is controlled by the application of pressure (positive or negative) and an appropriate time chosen by the user.

As described above, imaging of cell colonies growing in the wells of the SWIIN is desired in most implementations for, e.g., monitoring cell growth and device performance and imaging is necessary for phenotypic readout and either cherry-picking or ablation implementations. Real-time monitoring of cell growth or phenotypic readout in the SWIIN requires backlighting, retentate plate (top plate) condensation management and a system-level approach to temperature control, air flow, and thermal management. In some implementations, imaging employs a camera or CCD device with sufficient resolution to be able to image individual wells. For example, in some configurations a camera with a 9-pixel pitch is used (that is, there are 9 pixels center-to-center for each well). Processing the images may, in some implementations, utilize reading the images in grayscale, rating each pixel from low to high, where wells with no cells will be brightest (due to full or nearly-full light transmission from the backlight) and wells with cells will be dim (due to cells blocking light transmission from the backlight). After processing the images, thresholding is performed to determine which pixels will be called “bright” or “dim”, spot finding is performed to find bright pixels and arrange them into blocks, and then the spots are arranged on a hexagonal grid of pixels that correspond to the spots. Once arranged, the measure of intensity of each well is extracted, by, e.g., looking at one or more pixels in the middle of the spot, looking at several to many pixels at random or pre-set positions, or averaging X number of pixels in the spot. In addition, background intensity may be subtracted. Thresholding may be used to call each well positive (e.g., containing cells or comprising a desired phenotype) or negative (e.g., no cells in the well or not comprising a desired phenotype). The imaging information may be used in several ways, including taking images at time points for monitoring cell growth, identifying cells with a desired phenotype, recovering cells from specific wells or ablating cells with, e.g., UV light to eliminate cells that do not demonstrate the desired phenotype.

FIG. 3I depicts the embodiment of the SWIIN module in FIGS. 3A-3H further comprising a heat management system including a heater and a heated cover. The heater cover facilitates the condensation management that is required for imaging. Assembly 398 comprises a SWIIN module 350 seen lengthwise in cross section, where one permeate reservoir 352 is seen. Disposed immediately upon SWIIN module 350 is cover 394 and disposed immediately below SWIIN module 350 is backlight 380, which allows for imaging. Beneath and adjacent to the backlight and SWIIN module is insulation 382, which is disposed over a heatsink 384. In this FIG. 3I, the fins of the heatsink would be in-out of the page. In addition there is also axial fan 386 and heat sink 388, as well as two thermoelectric coolers 392, and a controller 390 to control the pneumatics, thermoelectric coolers, fan, solenoid valves, etc. The arrows denote cool air coming into the unit and hot air being removed from the unit. It should be noted that control of heating allows for growth of many different types of cells (prokaryotic and eukaryotic) as well as strains of cells that are, e.g., temperature sensitive, etc., and allows use of temperature-sensitive promoters. Temperature control allows for protocols to be adjusted to account for differences in transformation efficiency, cell growth and viability.

FIG. 3J is an exemplary pneumatic block diagram suitable for the SWIIN module depicted in FIGS. 3A-3I. In this configuration, there are two manifold arms that are controlled independently and there are two proportional valves, one each for the manifold arms. Tables 1-3 relate to the pneumatic diagram in FIG. 3J. Table 1 lists, for each step 1-32, the manifold arm status (open=arm open, closed=arm closed, motor engaged for pressurization); pump status (1: on, 0: off); energy status (1: energized, 0: de-energized) for each solenoid valve 1-4; and the pressure in psi for each proportional valve. Table 2 lists, for each step 1-32, the detection and threshold status for flow meters 1 and 2 as well as the duration of each step. When a change in pressure precedes a valve event, there is a delay of 1 second after reaching the set point before energizing the valves to avoid applying over- and under-shoots to the system. FALL=monitor for a falling signal, RISE=monitor for a rising signal. “Requires pLLD”=requires pressure-driven liquid level detection, such as, e.g., via air-displacement pipettor. Table 3 lists, for each step 1-32, the volumes for each reservoir, permeate reservoirs 1 and 2, and retentate reservoirs 1 and 2; the temperature of the SWIIN; and notes for operation.

### Automated Cell Editing Instruments and Modules

### Automated Cell Editing Instruments

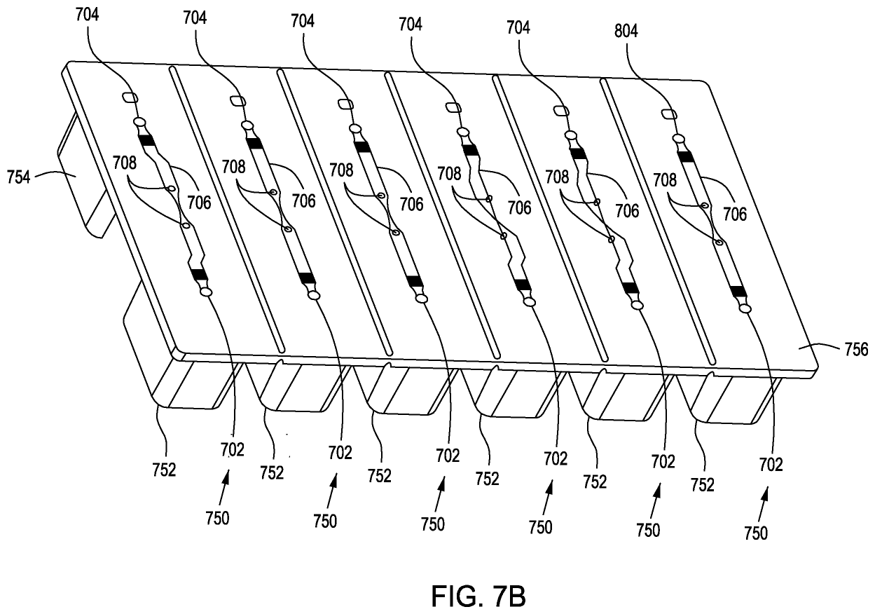

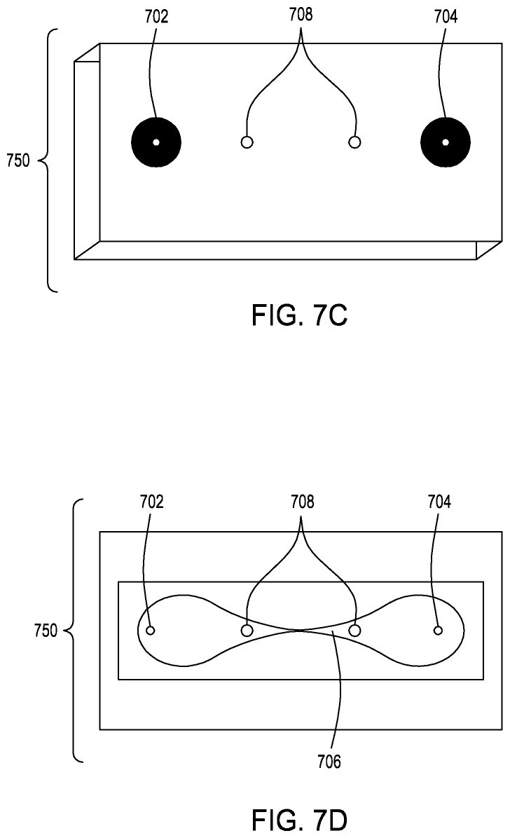

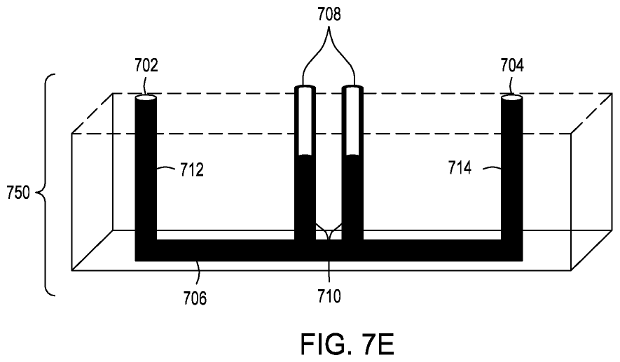

FIG. 4A depicts an exemplary stand-alone automated multi-module cell processing instrument 400 to, e.g., perform one of the exemplary workflows described infra, where the automated multi-module cell processing instrument performs the processes of cell growth, cell concentration and buffer exchange to render the cells electrocompetent, cell transformation, cell selection, and cell editing all without human intervention. The instrument 400, for example, may be and preferably is designed as a stand-alone desktop instrument for use within a laboratory environment. The instrument 400 may incorporate a mixture of reusable and disposable components for performing the various integrated processes in conducting automated genome cleavage and/or editing in cells. Illustrated is a gantry 402, providing an automated mechanical motion system (actuator) (not shown) that supplies XYZ axis motion control to, e.g., an automated (i.e., robotic) liquid handling system 458 including, e.g., an air displacement pipettor 432 which allows for cell processing among multiple modules without human intervention. In some automated multi-module cell processing instruments, the air displacement pipettor 432 is moved by gantry 402 and the various modules and reagent cartridges remain stationary; however, in other embodiments, the liquid handling system 458 may stay stationary while the various modules and reagent cartridges are moved. Also included in the automated multi-module cell processing instrument 400 is reagent cartridge 410 comprising reservoirs 412 and transformation module 430 (e.g., a flow-through electroporation device as described in detail in relation to FIGS. 7A-7E), as well as a wash cartridge 404 comprising reservoirs 406. The wash cartridge 404 may be configured to accommodate large tubes, for example, wash solutions, or solutions that are used often throughout an iterative process. In one example, wash cartridge 404 may be configured to remain in place when two or more reagent cartridges 410 are sequentially used and replaced. Although reagent cartridge 410 and wash cartridge 404 are shown in FIG. 4A as separate cartridges, the contents of wash cartridge 404 may be incorporated into reagent cartridge 410. The reagent cartridge 410 and wash cartridge 404 may be identical except for the consumables (reagents or other components contained within the various inserts) inserted therein. Note in this embodiment transformation module 430 is contained within reagent cartridge 410; however, in alternative embodiments transformation module 430 is contained within its own module or may be part of another module, such as a growth module.

In some implementations, the wash and reagent cartridges 404 and 410 comprise disposable kits (one or more of the various inserts and reagents) provided for use in the automated multi-module cell processing/editing instrument 400. For example, a user may open and position each of the reagent cartridge 410 and the wash cartridge 404 comprising various desired inserts and reagents within a chassis of the automated multi-module cell editing instrument 400 prior to activating cell processing.

Also illustrated in FIG. 4A is the robotic liquid handling system 458 including the gantry 402 and air displacement pipettor 432. In some examples, the robotic handling system 458 may include an automated liquid handling system such as those manufactured by Tecan Group Ltd. of Mannedorf, Switzerland, Hamilton Company of Reno, Nev. (see, e.g., WO2018015544A1), or Beckman Coulter, Inc. of Fort Collins, Colo. (see, e.g., US20160018427A1). Pipette tips may be provided in a pipette transfer tip supply (not shown) for use with the air displacement pipettor 432.

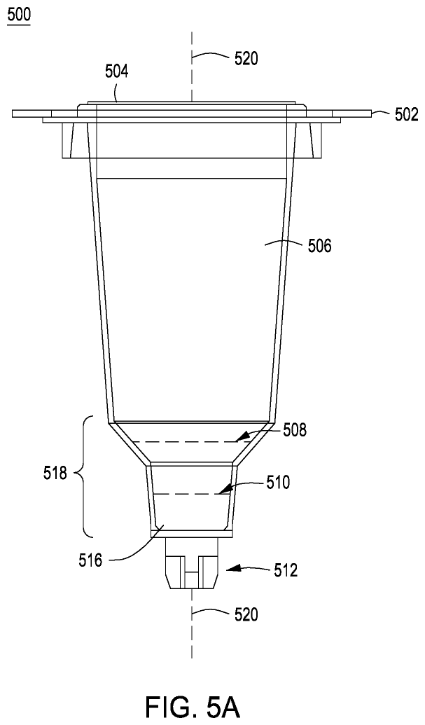

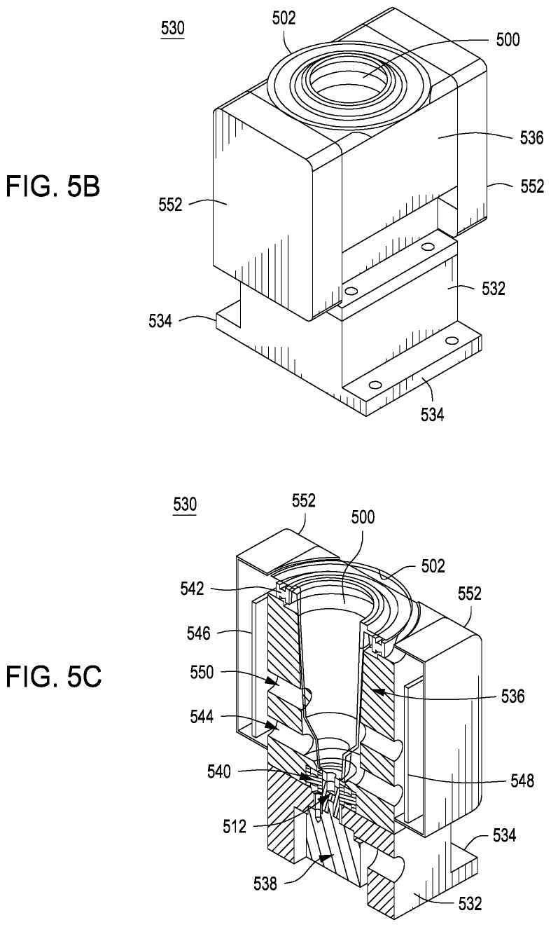

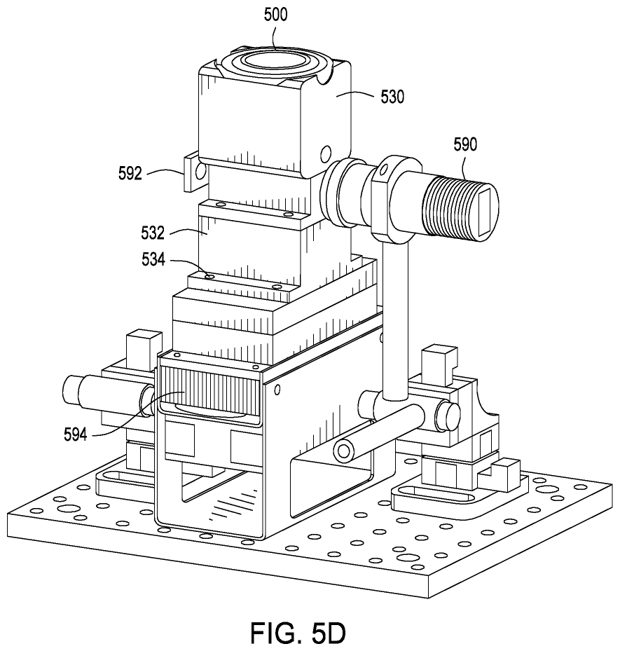

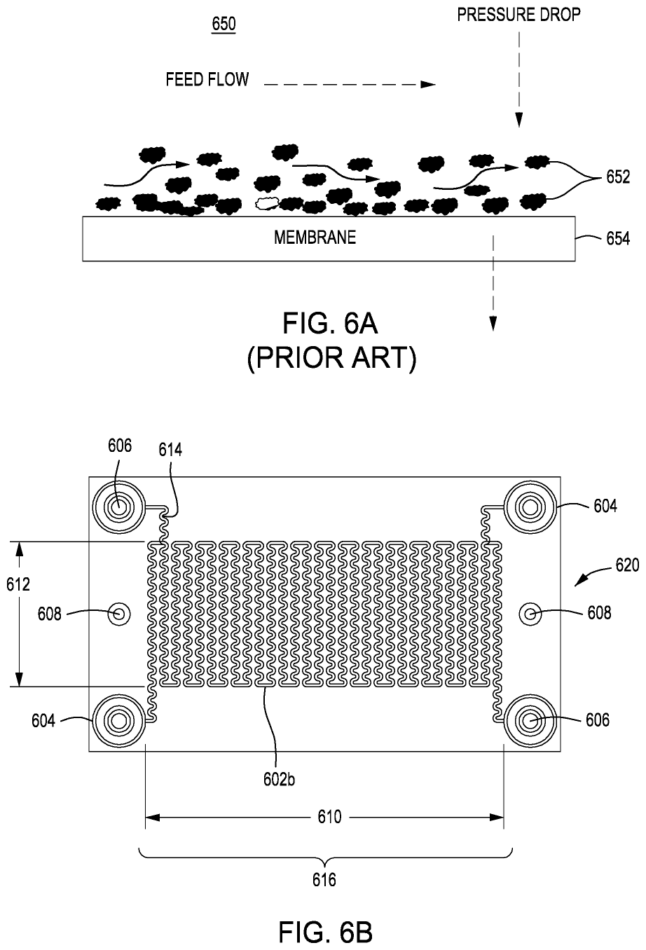

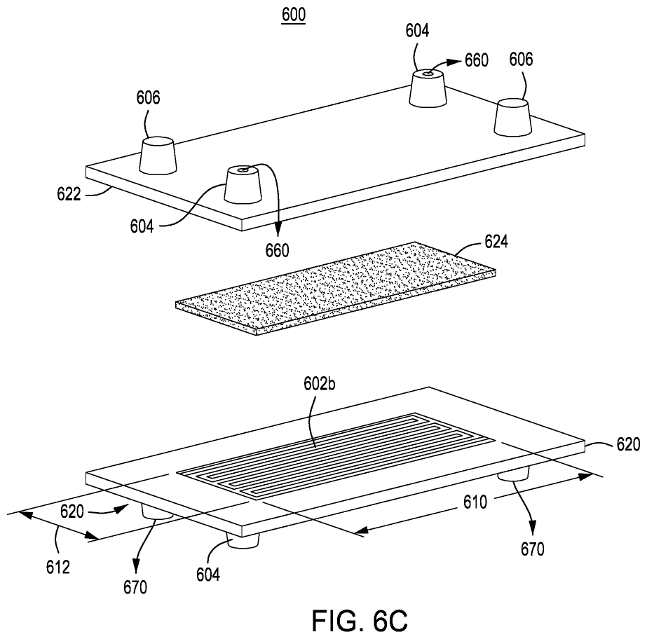

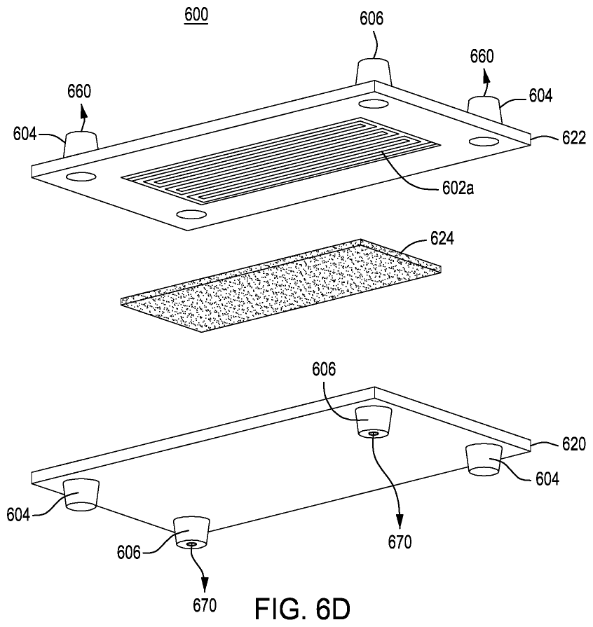

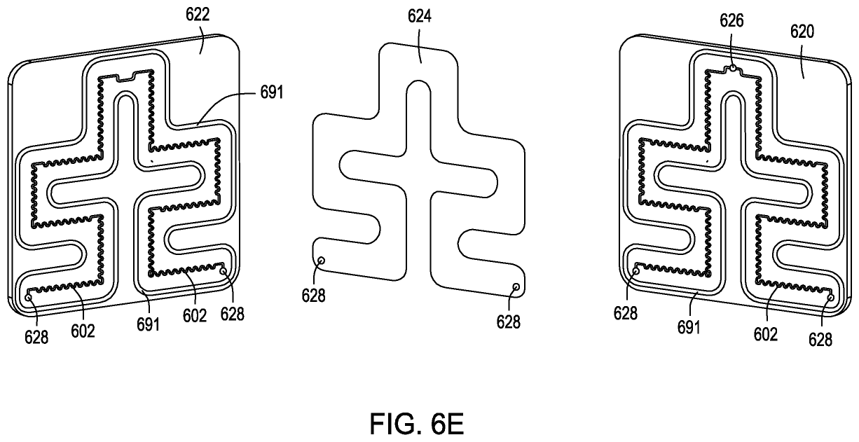

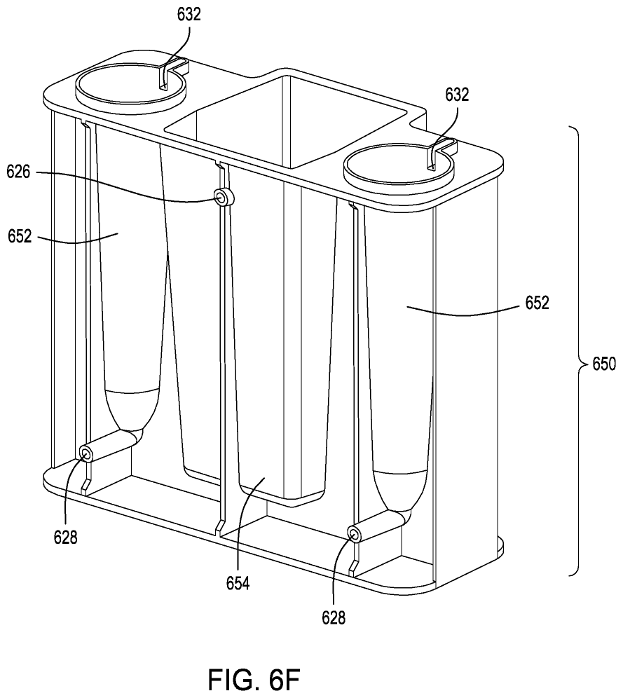

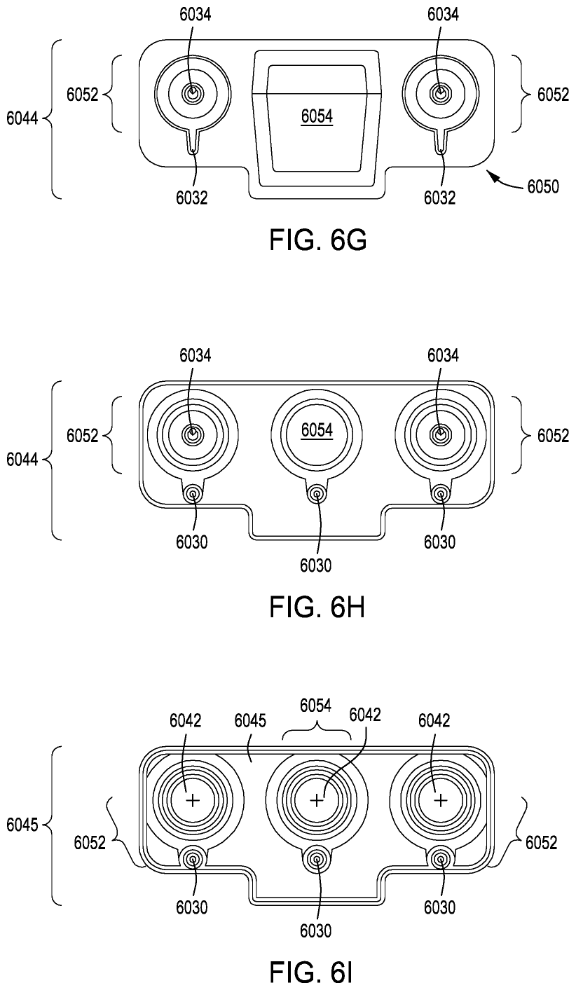

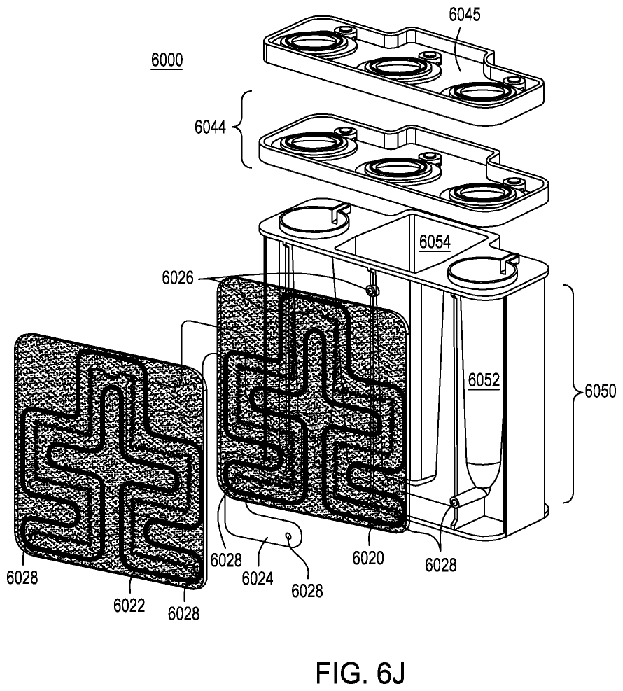

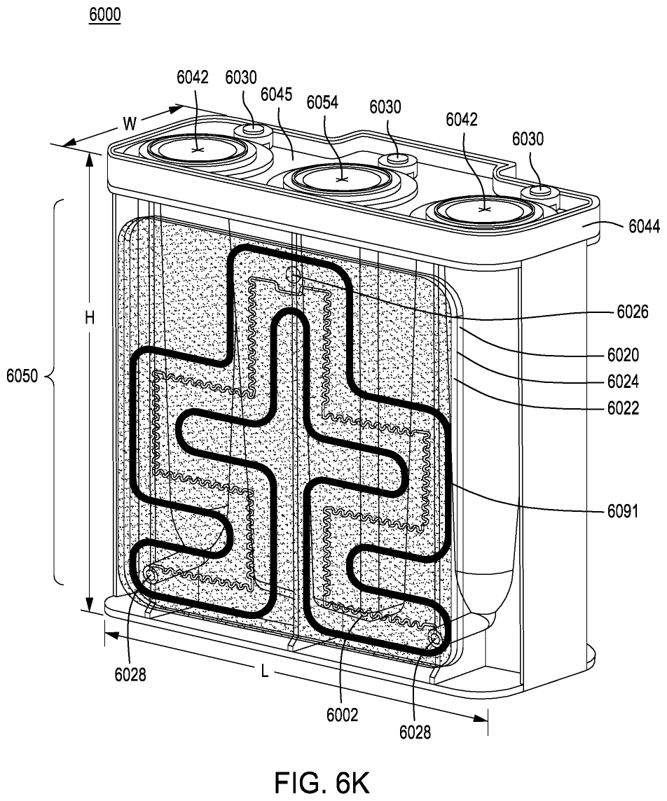

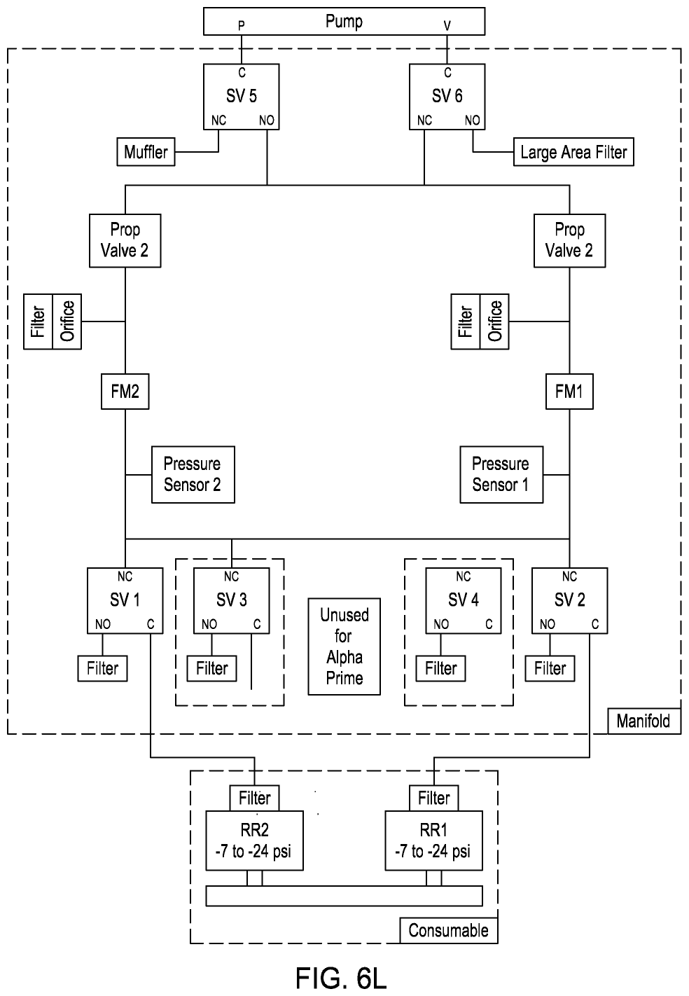

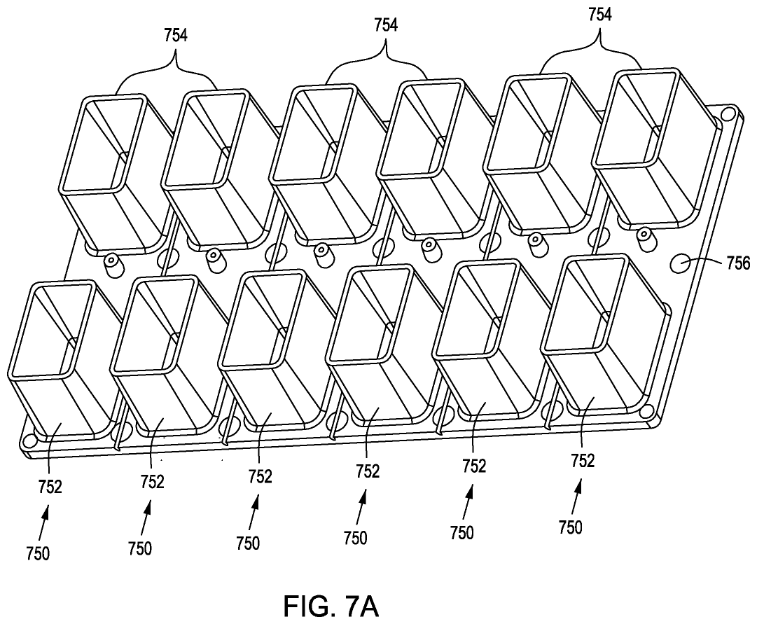

Inserts or components of the wash and reagent cartridges 404, 410, in some implementations, are marked with machine-readable indicia (not shown), such as bar codes, for recognition by the robotic handling system 458. For example, the robotic liquid handling system 458 may scan one or more inserts within each of the wash and reagent cartridges 404, 410 to confirm contents. In other implementations, machine-readable indicia may be marked upon each wash and reagent cartridge 404, 410, and a processing system (not shown, but see element 426 of FIG. 4B) of the automated multi-module cell editing instrument 400 may identify a stored materials map based upon the machine-readable indicia. The exemplary automated multi-module cell processing instrument 400 of FIG. 4A further comprises a cell growth module 434. (Note, all modules recited briefly here are described in greater detail below.) In the embodiment illustrated in FIG. 4A, the cell growth module 434 comprises two cell growth vials 418, 420 (described in greater detail below in relation to FIGS. 5A-5D) as well as a cell concentration module 422 (described in detail in relation to FIGS. 6A-6K). In alternative embodiments, the cell concentration module 422 may be separate from cell growth module 434, e.g., in a separate, dedicated module. Also illustrated as part of the automated multi-module cell processing instrument 400 of FIG. 4A is an isolation module 440, served by, e.g., robotic liquid handing system 458 and air displacement pipettor 432. Also seen are an optional nucleic acid assembly/desalting module 414 comprising a reaction chamber or tube receptacle (not shown) and a magnet 416 to allow for purification of nucleic acids using, e.g., magnetic solid phase reversible immobilization (SPRI) beads (Applied Biological Materials Inc., Richmond, BC. The cell growth module, cell concentration module, transformation module, reagent cartridge, and nucleic acid assembly module are described in greater detail infra, and an exemplary isolation module (which may also serve as a recovery and growth module as well as an incubation and normalization module) is described in detail in relation to FIGS. 2A-2B and 3A-3J supra.

FIG. 4B is a plan view of the front of the exemplary multi-module cell processing instrument 400 depicted in FIG. 4A. Cartridge-based source materials (such as in reagent cartridge 410), for example, may be positioned in designated areas on a deck of the instrument 400 for access by a robotic handling instrument (not shown in this figure). As illustrated in FIG. 4B, the deck may include a protection sink 403 such that contaminants spilling, dripping, or overflowing from any of the modules of the instrument 400 are contained within a lip of the protection sink 403. In addition to reagent cartridge 410, also seen in FIG. 4B is wash cartridge 404, isolation module 440, and a portion of growth module 434. Also seen in this view is touch screen display 450, transformation module controls 438, electronics rack 436, and processing system 426.

FIGS. 4C and 4D illustrate side and front views, respectively, of multi-module cell processing instrument 400 comprising chassis 490 for use in desktop versions of the automated multi-module cell editing instrument 400. For example, the chassis 490 may have a width of about 24-48 inches, a height of about 24-48 inches and a depth of about 24-48 inches. Chassis 490 may be and preferably is designed to hold all modules and disposable supplies used in automated cell processing and to perform all processes required without human intervention (that is, chassis 490 is configured to provide an integrated, stand-alone automated multi-module cell processing instrument). Chassis 490 may mount a robotic liquid handling system 458 for moving materials between modules. As illustrated in FIG. 4C, the chassis 490 includes a cover 452 having a handle 454 and hinge 456a (hinges 456b and 456c are seen in FIG. 4D) for lifting the cover 452 and accessing the interior of the chassis 490. A cooling grate 464 (FIG. 4C) allows for air flow via an internal fan (not shown). Further, the chassis 490 is lifted by adjustable feet 470a, 470c (feet 470, 470b are shown in FIG. 4D). Adjustable feet 470a-470c, for example, may provide additional air flow beneath the chassis 490. A control button 466, in some embodiments, allows for single-button automated start and/or stop of cell processing within the automated multi-module cell processing instrument 400.

Inside the chassis 490, in some implementations, a robotic liquid handling system 458 is disposed along a gantry 402 above wash cartridge 404 (reagent cartridge 410 is not seen in these figures). Control circuitry, liquid handling tubes, air pump controls, valves, thermal units (e.g., heating and cooling units) and other control mechanisms, in some embodiments, are disposed below a deck of the chassis 490, in a control box region 468. Also seen in both FIGS. 4C and 4D is isolation device or module 440. Nucleic acid assembly module 414 comprising a magnet 416 is seen in FIG. 4D.

Although not illustrated, in some embodiments a display screen may be positioned on the front face of the chassis 490, for example covering a portion of the cover (e.g., see display 450 in FIG. 4B). The display screen may provide information to the user regarding the processing status of the automated multi-module cell editing instrument 400. In another example, the display screen may accept inputs from the user for conducting the cell processing.

### The Rotating Growth Module

FIG. 5A shows one embodiment of a rotating growth vial 500 for use with the cell growth device described herein. The rotating growth vial 500 is an optically-transparent container having an open end 504 for receiving liquid media and cells, a central vial region 506 that defines the primary container for growing cells, a tapered-to-constricted region 518 defining at least one light path 510, a closed end 516, and a drive engagement mechanism 512. The rotating growth vial 500 has a central longitudinal axis 520 around which the vial rotates, and the light path 510 is generally perpendicular to the longitudinal axis of the vial. The first light path 510 is positioned in the lower constricted portion of the tapered-to-constricted region 518. Optionally, some embodiments of the rotating growth vial 500 have a second light path 508 in the tapered region of the tapered-to-constricted region 518. Both light paths in this embodiment are positioned in a region of the rotating growth vial that is constantly filled with the cell culture (cells+growth media) and are not affected by the rotational speed of the growth vial. The first light path 510 is shorter than the second light path 508 allowing for sensitive measurement of OD values when the OD values of the cell culture in the vial are at a high level (e.g., later in the cell growth process), whereas the second light path 508 allows for sensitive measurement of OD values when the OD values of the cell culture in the vial are at a lower level (e.g., earlier in the cell growth process).

The drive engagement mechanism 512 engages with a motor (not shown) to rotate the vial. In some embodiments, the motor drives the drive engagement mechanism 512 such that the rotating growth vial 500 is rotated in one direction only, and in other embodiments, the rotating growth vial 500 is rotated in a first direction for a first amount of time or periodicity, rotated in a second direction (i.e., the opposite direction) for a second amount of time or periodicity, and this process may be repeated so that the rotating growth vial 500 (and the cell culture contents) are subjected to an oscillating motion. Further, the choice of whether the culture is subjected to oscillation and the periodicity therefor may be selected by the user. The first amount of time and the second amount of time may be the same or may be different. The amount of time may be 1, 2, 3, 4, 5, or more seconds, or may be 1, 2, 3, 4 or more minutes. In another embodiment, in an early stage of cell growth the rotating growth vial 500 may be oscillated at a first periodicity (e.g., every 60 seconds), and then a later stage of cell growth the rotating growth vial 500 may be oscillated at a second periodicity (e.g., every one second) different from the first periodicity.

The rotating growth vial 500 may be reusable or, preferably, the rotating growth vial is consumable. In some embodiments, the rotating growth vial is consumable and is presented to the user pre-filled with growth medium, where the vial is hermetically sealed at the open end 504 with a foil seal. A medium-filled rotating growth vial packaged in such a manner may be part of a kit for use with a stand-alone cell growth device or with a cell growth module that is part of an automated multi-module cell processing system. To introduce cells into the vial, a user need only pipette up a desired volume of cells and use the pipette tip to punch through the foil seal of the vial. Open end 504 may optionally include an extended lip 502 to overlap and engage with the cell growth device. In automated systems, the rotating growth vial 500 may be tagged with a barcode or other identifying means that can be read by a scanner or camera (not shown) that is part of the automated system.

The volume of the rotating growth vial 500 and the volume of the cell culture (including growth medium) may vary greatly, but the volume of the rotating growth vial 500 must be large enough to generate a specified total number of cells. In practice, the volume of the rotating growth vial 500 may range from 1-250 mL, 2-100 mL, from 5-80 mL, 10-50 mL, or from 12-35 mL. Likewise, the volume of the cell culture (cells+growth media) should be appropriate to allow proper aeration and mixing in the rotating growth vial 500. Proper aeration promotes uniform cellular respiration within the growth media. Thus, the volume of the cell culture should be approximately 5-85% of the volume of the growth vial or from 20-60% of the volume of the growth vial. For example, for a 30 mL growth vial, the volume of the cell culture would be from about 1.5 mL to about 26 mL, or from 6 mL to about 18 mL.

The rotating growth vial 500 preferably is fabricated from a bio-compatible optically transparent material—or at least the portion of the vial comprising the light path(s) is transparent. Additionally, material from which the rotating growth vial is fabricated should be able to be cooled to about 4° C. or lower and heated to about 55° C. or higher to accommodate both temperature-based cell assays and long-term storage at low temperatures. Further, the material that is used to fabricate the vial must be able to withstand temperatures up to 55° C. without deformation while spinning. Suitable materials include cyclic olefin copolymer (COC), glass, polyvinyl chloride, polyethylene, polyamide, polypropylene, polycarbonate, poly(methyl methacrylate) (PMMA), polysulfone, polyurethane, and co-polymers of these and other polymers. Preferred materials include polypropylene, polycarbonate, or polystyrene. In some embodiments, the rotating growth vial is inexpensively fabricated by, e.g., injection molding or extrusion.

FIG. 5B is a perspective view of one embodiment of a cell growth device 530. FIG. 5C depicts a cut-away view of the cell growth device 530 from FIG. 5B. In both figures, the rotating growth vial 500 is seen positioned inside a main housing 536 with the extended lip 502 of the rotating growth vial 500 extending above the main housing 536. Additionally, end housings 552, a lower housing 532 and flanges 534 are indicated in both figures. Flanges 534 are used to attach the cell growth device 530 to heating/cooling means or other structure (not shown). FIG. 5C depicts additional detail. In FIG. 5C, upper bearing 542 and lower bearing 540 are shown positioned within main housing 536. Upper bearing 542 and lower bearing 540 support the vertical load of rotating growth vial 500. Lower housing 532 contains the drive motor 538. The cell growth device 530 of FIG. 5C comprises two light paths: a primary light path 544, and a secondary light path 550. Light path 544 corresponds to light path 510 positioned in the constricted portion of the tapered-to-constricted portion of the rotating growth vial 500, and light path 550 corresponds to light path 508 in the tapered portion of the tapered-to-constricted portion of the rotating growth via 500. Light paths 510 and 508 are not shown in FIG. 5C but may be seen in FIG. 5A. In addition to light paths 544 and 540, there is an emission board 548 to illuminate the light path(s), and detector board 546 to detect the light after the light travels through the cell culture liquid in the rotating growth vial 500.

The motor 538 engages with drive mechanism 512 and is used to rotate the rotating growth vial 500. In some embodiments, motor 538 is a brushless DC type drive motor with built-in drive controls that can be set to hold a constant revolution per minute (RPM) between 0 and about 3000 RPM. Alternatively, other motor types such as a stepper, servo, brushed DC, and the like can be used. Optionally, the motor 538 may also have direction control to allow reversing of the rotational direction, and a tachometer to sense and report actual RPM. The motor is controlled by a processor (not shown) according to, e.g., standard protocols programmed into the processor and/or user input, and the motor may be configured to vary RPM to cause axial precession of the cell culture thereby enhancing mixing, e.g., to prevent cell aggregation, increase aeration, and optimize cellular respiration.

Main housing 536, end housings 552 and lower housing 532 of the cell growth device 530 may be fabricated from any suitable, robust material including aluminum, stainless steel, and other thermally conductive materials, including plastics. These structures or portions thereof can be created through various techniques, e.g., metal fabrication, injection molding, creation of structural layers that are fused, etc. Whereas the rotating growth vial 500 is envisioned in some embodiments to be reusable, but preferably is consumable, the other components of the cell growth device 530 are preferably reusable and function as a stand-alone benchtop device or as a module in a multi-module cell processing system.

The processor (not shown) of the cell growth device 530 may be programmed with information to be used as a “blank” or control for the growing cell culture. A “blank” or control is a vessel containing cell growth medium only, which yields 100% transmittance and 0 OD, while the cell sample will deflect light rays and will have a lower percent transmittance and higher OD. As the cells grow in the media and become denser, transmittance will decrease and OD will increase. The processor (not shown) of the cell growth device 530—may be programmed to use wavelength values for blanks commensurate with the growth media typically used in cell culture (whether, e.g., mammalian cells, bacterial cells, animal cells, yeast cells, etc.). Alternatively, a second spectrophotometer and vessel may be included in the cell growth device 530, where the second spectrophotometer is used to read a blank at designated intervals.

FIG. 5D illustrates a cell growth device 530 as part of an assembly comprising the cell growth device 530 of FIG. 5B coupled to light source 590, detector 592, and thermal components 594. The rotating growth vial 500 is inserted into the cell growth device. Components of the light source 590 and detector 592 (e.g., such as a photodiode with gain control to cover 5-log) are coupled to the main housing of the cell growth device. The lower housing 532 that houses the motor that rotates the rotating growth vial 500 is illustrated, as is one of the flanges 534 that secures the cell growth device 530 to the assembly. Also, the thermal components 594 illustrated are a Peltier device or thermoelectric cooler. In this embodiment, thermal control is accomplished by attachment and electrical integration of the cell growth device 530 to the thermal components 594 via the flange 534 on the base of the lower housing 532. Thermoelectric coolers are capable of “pumping” heat to either side of a junction, either cooling a surface or heating a surface depending on the direction of current flow. In one embodiment, a thermistor is used to measure the temperature of the main housing and then, through a standard electronic proportional-integral-derivative (PID) controller loop, the rotating growth vial 500 is controlled to approximately +/−0.5° C.

In use, cells are inoculated (cells can be pipetted, e.g., from an automated liquid handling system or by a user) into pre-filled growth media of a rotating growth vial 500 by piercing though the foil seal or film. The programmed software of the cell growth device 530 sets the control temperature for growth, typically 30° C., then slowly starts the rotation of the rotating growth vial 500. The cell/growth media mixture slowly moves vertically up the wall due to centrifugal force allowing the rotating growth vial 500 to expose a large surface area of the mixture to a normal oxygen environment. The growth monitoring system takes either continuous readings of the OD or OD measurements at pre-set or pre-programmed time intervals. These measurements are stored in internal memory and if requested the software plots the measurements versus time to display a growth curve. If enhanced mixing is required, e.g., to optimize growth conditions, the speed of the vial rotation can be varied to cause an axial precession of the liquid, and/or a complete directional change can be performed at programmed intervals. The growth monitoring can be programmed to automatically terminate the growth stage at a pre-determined OD, and then quickly cool the mixture to a lower temperature to inhibit further growth.

One application for the cell growth device 530 is to constantly measure the optical density of a growing cell culture. One advantage of the described cell growth device is that optical density can be measured continuously (kinetic monitoring) or at specific time intervals; e.g., every 5, 10, 15, 20, 30 45, or 60 seconds, or every 1, 2, 3, 4, 5, 6, 7, 8, 9, or 10 minutes. While the cell growth device 530 has been described in the context of measuring the optical density (OD) of a growing cell culture, it should, however, be understood by a skilled artisan given the teachings of the present specification that other cell growth parameters can be measured in addition to or instead of cell culture OD. As with optional measure of cell growth in relation to the solid wall device or module described supra, spectroscopy using visible, UV, or near infrared (NIR) light allows monitoring the concentration of nutrients and/or wastes in the cell culture and other spectroscopic measurements may be made; that is, other spectral properties can be measured via, e.g., dielectric impedance spectroscopy, visible fluorescence, fluorescence polarization, or luminescence. Additionally, the cell growth device 630 may include additional sensors for measuring, e.g., dissolved oxygen, carbon dioxide, pH, conductivity, and the like.

### Cell Concentration Module

FIGS. 6A-6K depict variations on one embodiment of a cell concentration/buffer exchange cassette and module that utilizes tangential flow filtration. One embodiment of a cell concentration device described herein operates using tangential flow filtration (TFF), also known as crossflow filtration, in which the majority of the feed flows tangentially over the surface of the filter thereby reducing cake (retentate) formation as compared to dead-end filtration, in which the feed flows into the filter. Secondary flows relative to the main feed are also exploited to generate shear forces that prevent filter cake formation and membrane fouling thus maximizing particle recovery, as described below.

The TFF device described herein was designed to take into account two primary design considerations. First, the geometry of the TFF device leads to filtering the cell culture over a large surface area so as to minimize processing time. Second, the design of the TFF device is configured to minimize filter fouling. FIG. 6A is a general model 650 of tangential flow filtration. The TFF device operates using tangential flow filtration, also known as cross-flow filtration. FIG. 6A shows cells flowing over a membrane 654, where the feed flow of the cells 652 in medium or buffer is parallel to the membrane 654. TFF is different from dead-end filtration where both the feed flow and the pressure drop are perpendicular to a membrane or filter.

FIG. 6B depicts a top view of the lower member of one embodiment of a TFF device/module providing tangential flow filtration. As can be seen in the embodiment of the TFF device of FIG. 6B, TFF device 600 comprises a channel structure 616 comprising a flow channel 602b through which a cell culture is flowed. The channel structure 616 comprises a single flow channel 602b that is horizontally bifurcated by a membrane (not shown) through which buffer or medium may flow, but cells cannot. This particular embodiment comprises an undulating serpentine geometry 614 (i.e., the small “wiggles” in the flow channel 602) and a serpentine “zig-zag” pattern where the flow channel 602 crisscrosses the device from one end at the left of the device to the other end at the right of the device. The serpentine pattern allows for filtration over a high surface area relative to the device size and total channel volume, while the undulating contribution creates a secondary inertial flow to enable effective membrane regeneration preventing membrane fouling. Although an undulating geometry and serpentine pattern are exemplified here, other channel configurations may be used as long as the channel can be bifurcated by a membrane, and as long as the channel configuration provides for flow through the TFF module in alternating directions. In addition to the flow channel 602b, portals 604 and 606 as part of the channel structure 616 can be seen, as well as recesses 608. Portals 604 collect cells passing through the channel on one side of a membrane (not shown) (the “retentate”), and portals 606 collect the medium (“filtrate” or “permeate”) passing through the channel on the opposite side of the membrane (not shown). In this embodiment, recesses 608 accommodate screws or other fasteners (not shown) that allow the components of the TFF device to be secured to one another.

The length 610 and width 612 of the channel structure 616 may vary depending on the volume of the cell culture to be grown and the optical density of the cell culture to be concentrated. The length 610 of the channel structure 616 typically is from 1 mm to 300 mm, or from 50 mm to 250 mm, or from 60 mm to 200 mm, or from 70 mm to 150 mm, or from 80 mm to 100 mm. The width of the channel structure 716 typically is from 1 mm to 120 mm, or from 20 mm to 100 mm, or from 30 mm to 80 mm, or from 40 mm to 70 mm, or from 50 mm to 60 mm. The cross-section configuration of the flow channel 702 may be round, elliptical, oval, square, rectangular, trapezoidal, or irregular. If square, rectangular, or another shape with generally straight sides, the cross section may be from about 10 μm to 1000 μm wide, or from 200 μm to 800 μm wide, or from 300 μm to 700 μm wide, or from 400 μm to 600 μm wide; and from about 10 μm to 1000 μm high, or from 200 μm to 800 μm high, or from 300 μm to 700 μm high, or from 400 μm to 600 μm high. If the cross section of the flow channel 602 is generally round, oval or elliptical, the radius of the channel may be from about 50 μm to 1000 μm in hydraulic radius, or from 5 μm to 800 μm in hydraulic radius, or from 200 μm to 700 μm in hydraulic radius, or from 300 μm to 600 μm wide in hydraulic radius, or from about 200 to 500 μm in hydraulic radius.

When looking at the top view of the TFF device/module of FIG. 6B, note that there are two retentate portals 604 and two filtrate portals 606, where there is one of each type portal at both ends (e.g., the narrow edge) of the device 600. In other embodiments, retentate and filtrate portals can on the same surface of the same member (e.g., upper or lower member), or they can be arranged on the side surfaces of the assembly. Unlike other TFF devices that operate continuously, the TFF device/module described herein uses an alternating method for concentrating cells. The overall work flow for cell concentration using the TFF device/module involves flowing a cell culture or cell sample tangentially through the channel structure. The membrane bifurcating the flow channels retains the cells on one side of the membrane and allows unwanted medium or buffer to flow across the membrane into a filtrate side (e.g., lower member 620) of the device. In this process, a fixed volume of cells in medium or buffer is driven through the device until the cell sample is collected into one of the retentate portals 604, and the medium/buffer that has passed through the membrane is collected through one or both of the filtrate portals 606. All types of prokaryotic and eukaryotic cells—both adherent and non-adherent cells—can be grown in the TFF device. Adherent cells may be grown on beads or other cell scaffolds suspended in medium that flow through the TFF device.

In the cell concentration process, passing the cell sample through the TFF device and collecting the cells in one of the retentate portals 604 while collecting the medium in one of the filtrate portals 606 is considered “one pass” of the cell sample. The transfer between retentate reservoirs “flips” the culture, The retentate and filtrate portals collecting the cells and medium, respectively, for a given pass reside on the same end of TFF device/module 600 with fluidic connections arranged so that there are two distinct flow layers for the retentate and filtrate sides, but if the retentate portal 604 resides on the upper member of device/module 600 (that is, the cells are driven through the channel above the membrane and the filtrate (medium) passes to the portion of the channel below the membrane), the filtrate portal 606 will reside on the lower member of device/module 600 and vice versa (that is, if the cell sample is driven through the channel below the membrane, the filtrate (medium) passes to the portion of the channel above the membrane). This configuration can be seen more clearly in FIGS. 6C-6D, where the retentate flows 660 from the retentate portals 604 and the filtrate flows 670 from the filtrate portals 606.

At the conclusion of a “pass” in the growth concentration process, the cell sample is collected by passing through the retentate portal 604 and into the retentate reservoir (not shown). To initiate another “pass”, the cell sample is passed again through the TFF device, this time in a flow direction that is reversed from the first pass. The cell sample is collected by passing through the retentate portal 604 and into retentate reservoir (not shown) on the opposite end of the device/module from the retentate portal 704 that was used to collect cells during the first pass. Likewise, the medium/buffer that passes through the membrane on the second pass is collected through the filtrate portal 606 on the opposite end of the device/module from the filtrate portal 606 that was used to collect the filtrate during the first pass, or through both portals. This alternating process of passing the retentate (the concentrated cell sample) through the device/module is repeated until the cells have been concentrated to a desired volume, and both filtrate portals can be open during the passes to reduce operating time. In addition, buffer exchange may be effected by adding a desired buffer (or fresh medium) to the cell sample in the retentate reservoir, before initiating another “pass”, and repeating this process until the old medium or buffer is diluted and filtered out and the cells reside in fresh medium or buffer. Note that buffer exchange and cell concentration may (and typically do) take place simultaneously.

FIG. 6C depicts a top view of upper (622) and lower (620) members of an exemplary TFF module. Again, portals 604 and 606 are seen. As noted above, recesses—such as the recesses 608 seen in FIG. 6B—provide a means to secure the components (upper member 622, lower member 620, and membrane 624) of the TFF device/membrane to one another during operation via, e.g., screws or other like fasteners. However, in alterative embodiments an adhesive, such as a pressure sensitive adhesive, or ultrasonic welding, or solvent bonding, may be used to couple the upper member 622, lower member 620, and membrane 624 together. Indeed, one of ordinary skill in the art given the guidance of the present disclosure can find yet other configurations for coupling the components of the TFF device, such as e.g., clamps; mated fittings disposed on the upper and lower members; combination of adhesives, welding, solvent bonding, and mated fittings; and other such fasteners and couplings.

Note that there is one retentate portal and one filtrate portal on each “end” (e.g., the narrow edges) of the TFF device/module. The retentate and filtrate portals on the left side of the device/module will collect cells (flow path at 660) and medium (flow path at 670), respectively, for the same pass. Likewise, the retentate and filtrate portals on the right side of the device/module will collect cells (flow path at 660) and medium (flow path at 670), respectively, for the same pass. In this embodiment, the retentate is collected from portals 604 on the top surface of the TFF device, and filtrate is collected from portals 606 on the bottom surface of the device. The cells are maintained in the TFF flow channel above the membrane 624, while the filtrate (medium) flows through membrane 624 and then through portals 606; thus, the top/retentate portals and bottom/filtrate portals configuration is practical. It should be recognized, however, that other configurations of retentate and filtrate portals may be implemented such as positioning both the retentate and filtrate portals on the side (as opposed to the top and bottom surfaces) of the TFF device. In FIG. 6C, the channel structure 602b can be seen on the bottom member 620 of the TFF device 600. However, in other embodiments, retentate and filtrate portals can reside on the same of the TFF device.

Also seen in FIG. 6C is membrane or filter 624. Filters or membranes appropriate for use in the TFF device/module are those that are solvent resistant, are contamination free during filtration, and are able to retain the types and sizes of cells of interest. For example, in order to retain small cell types such as bacterial cells, pore sizes can be as low as 0.2 μm, however for other cell types, the pore sizes can be as high as 5 μm. Indeed, the pore sizes useful in the TFF device/module include filters with sizes from 0.20 μm, 0.21 μm, 0.22 μm, 0.23 μm, 0.24 μm, 0.25 μm, 0.26 μm, 0.27 μm, 0.28 μm, 0.29 μm, 0.30 μm, 0.31 μm, 0.32 μm, 0.33 μm, 0.34 μm, 0.35 μm, 0.36 μm, 0.37 μm, 0.38 μm, 0.39 μm, 0.40 μm, 0.41 μm, 0.42 μm, 0.43 μm, 0.44 μm, 0.45 μm, 0.46 μm, 0.47 μm, 0.48 μm, 0.49 μm, 0.50 μm and larger. The filters may be fabricated from any suitable non-reactive material including cellulose mixed ester (cellulose nitrate and acetate) (CME), polycarbonate (PC), polyvinylidene fluoride (PVDF), polyethersulfone (PES), polytetrafluoroethylene (PTFE), nylon, glass fiber, or metal substrates as in the case of laser or electrochemical etching. The TFF device shown in FIGS. 6C and 6D do not show a seat in the upper 612 and lower 620 members where the filter 624 can be seated or secured (for example, a seat half the thickness of the filter in each of upper 612 and lower 620 members); however, such a seat is contemplated in some embodiments.

FIG. 6D depicts a bottom view of upper and lower components of the exemplary TFF module shown in FIG. 6C. FIG. 6D depicts a bottom view of upper (622) and lower (620) components of an exemplary TFF module. Again portals 604 and 606 are seen. Note again that there is one retentate portal and one filtrate portal on each end of the device/module. The retentate and filtrate portals on the left side of the device/module will collect cells (flow path at 660) and medium (flow path at 670), respectively, for the same pass. Likewise, the retentate and filtrate portals on the right side of the device/module will collect cells (flow path at 660) and medium (flow path at 670), respectively, for the same pass. In FIG. 6D, the channel structure 602a can be seen on the upper member 622 of the TFF device 600. Thus, looking at FIGS. 6C and 6D, note that there is a channel structure 602 (602a and 602b) in both the upper and lower members, with a membrane 624 between the upper and lower portions of the channel structure. The channel structure 602 of the upper 622 and lower 620 members (602a and 602b, respectively) mate to create the flow channel with the membrane 624 positioned horizontally between the upper and lower members of the flow channel thereby bifurcating the flow channel.

Medium exchange (during cell growth) or buffer exchange (during cell concentration or rendering the cells competent) is performed on the TFF device/module by adding fresh medium to growing cells or a desired buffer to the cells concentrated to a desired volume; for example, after the cells have been concentrated at least 20-fold, 30-fold, 40-fold, 50-fold, 60-fold, 70-fold, 80-fold, 90-fold, 100-fold, 150-fold, 200-fold or more. A desired exchange medium or exchange buffer is added to the cells either by addition to the retentate reservoir or thorough the membrane from the filtrate side and the process of passing the cells through the TFF device 600 is repeated until the cells have been grown to a desired optical density or concentrated to a desired volume in the exchange medium or buffer. This process can be repeated any number of desired times so as to achieve a desired level of exchange of the buffer and a desired volume of cells. The exchange buffer may comprise, e.g., glycerol or sorbitol thereby rendering the cells competent for transformation in addition to decreasing the overall volume of the cell sample.

The TFF device 600 may be fabricated from any robust material in which channels (and channel branches) may be milled including stainless steel, silicon, glass, aluminum, or plastics including cyclic-olefin copolymer (COC), cyclo-olefin polymer (COP), polystyrene, polyvinyl chloride, polyethylene, polyamide, polyethylene, polypropylene, acrylonitrile butadiene, polycarbonate, polyetheretheketone (PEEK), poly(methyl methylacrylate) (PMMA), polysulfone, and polyurethane, and co-polymers of these and other polymers. If the TFF device/module is disposable, preferably it is made of plastic. In some embodiments, the material used to fabricate the TFF device/module is thermally-conductive so that the cell culture may be heated or cooled to a desired temperature. In certain embodiments, the TFF device is formed by precision mechanical machining, laser machining, electro discharge machining (for metal devices); wet or dry etching (for silicon devices); dry or wet etching, powder or sandblasting, photostructuring (for glass devices); or thermoforming, injection molding, hot embossing, or laser machining (for plastic devices) using the materials mentioned above that are amenable to this mass production techniques.

FIGS. 6E-6K depict an alternative embodiment of a tangential flow filtration (TFF) device/module, where the module has the advantage of a reduced footprint, in, e.g., an automated multi-module cell processing instrument. FIG. 6E depicts a configuration of an upper (retentate) member 6022 (on left), a membrane or filter 6024 (middle), and a lower (permeate/filtrate) member 6020 (on the right). In the configuration shown in FIGS. 6E-6K, the retentate member 6022 is no longer “upper” and the permeate/filtrate member 6020 is no longer “lower”, as the retentate member 6022 and permeate/filtrate member 6020 are coupled side-to-side as seen in FIGS. 6J and 6K. In FIG. 6E, retentate member 6022 comprises a tangential flow channel 6002, which has a serpentine configuration that initiates at one lower corner of retentate member 6022—specifically at retentate port 6028—traverses across and up then down and across retentate member 6022, ending in the other lower corner of retentate member 6022 at a second retentate port 6028. Also seen on retentate member 6022 is energy director 6091, which circumscribes the region where membrane or filter 6024 is seated. Energy director 6091 in this embodiment mates with and serves to facilitate ultrasonic wending or bonding of retentate member 6022 with permeate/filtrate member 6020 via the energy director component on permeate/filtrate member 6020. Also seen is membrane or filter 6024 has through-holes for retentate ports 6028, which is configured to seat within the circumference of energy directors 6091 between the retentate member 6022 and the permeate/filtrate member 6020. Permeate/filtrate member 6020 comprises, in addition to energy director 6091, through-holes for retentate port 6028 at each bottom corner (which mate with the through-holes for retentate ports 6028 at the bottom corners of membrane 6024 and retentate ports 6028 in retentate member 6022), as well as a tangential flow channel 6002 and a single permeate/filtrate port 6026 positioned at the top and center of permeate/filtrate member 6020. The tangential flow channel 6002 structure in this embodiment has a serpentine configuration and an undulating geometry, although other geometries may be used. In some aspects, the length of the tangential flow channel is from 10 mm to 1000 mm, from 60 mm to 200 mm, or from 80 mm to 100 mm. In some aspects, the width of the channel structure is from 10 mm to 120 mm, from 40 mm to 70 mm, or from 50 mm to 60 mm. In some aspects, the cross section of the tangential flow channel 1202 is rectangular. In some aspects, the cross section of the tangential flow channel 1202 is 5 μm to 1000 μm wide and 5 μm to 1000 μm high, 300 μm to 700 μm wide and 300 μm to 700 μm high, or 400 μm to 600 μm wide and 400 μm to 600 μm high. In other aspects, the cross section of the tangential flow channel 1202 is circular, elliptical, trapezoidal, or oblong, and is 100 μm to 1000 μm in hydraulic radius, 300 μm to 700 μm in hydraulic radius, or 400 μm to 600 μm in hydraulic radius.

FIG. 6F is a side perspective view of a reservoir assembly 6050. The embodiment of FIG. 6F, the retentate member is separate from the reservoir assembly. Reservoir assembly 6050 comprises retentate reservoirs 6052 on either side of a single permeate reservoir 6054. Retentate reservoirs 6052 are used to contain the cells and medium as the cells are transferred through the cell concentration/growth device or module and into the retentate reservoirs during cell concentration and/or growth. Permeate/filtrate reservoir 6054 is used to collect the filtrate fluids removed from the cell culture during cell concentration, or old buffer or medium during cell growth. In the embodiment depicted in FIGS. 6E-6L, buffer or medium is supplied to the permeate/filtrate member from a reagent reservoir separate from the device module. Additionally seen in FIG. 6F are grooves 6032 to accommodate pneumatic ports (not seen), permeate/filtrate port 6026, and retentate port through-holes 6028. The retentate reservoirs are fluidically coupled to the retentate ports 6028, which in turn are fluidically coupled to the portion of the tangential flow channel disposed in the retentate member (not shown). The permeate/filtrate reservoir is fluidically coupled to the permeate/filtrate port 6026 which in turn are fluidically coupled to the portion of the tangential flow channel disposed in permeate/filtrate member (not shown), where the portions of the tangential flow channels are bifurcated by membrane (not shown). In embodiments including the present embodiment, up to 120 mL of cell culture can be grown and/or filtered, or up to 100 mL, 90 mL, 80 mL, 70 mL, 60 mL, 50 mL, 40 mL, 30 mL or 20 mL of cell culture can be grown and/or concentrated.

FIG. 6G depicts a top-down view of the reservoir assembly 6050 shown in FIG. 6F, FIG. 6H depicts a cover 6044 for reservoir assembly 6050 shown in FIGS. 6F, and 6I depicts a gasket 6045 that in operation is disposed on cover 6044 of reservoir assembly 6050 shown in FIG. 6F. FIG. 6G is a top-down view of reservoir assembly 6050, showing two retentate reservoirs 6052, one on either side of permeate reservoir 6054. Also seen are grooves 6032 that will mate with a pneumatic port (not shown), and fluid channels 6034 that reside at the bottom of retentate reservoirs 6052, which fluidically couple the retentate reservoirs 6052 with the retentate ports 6028 (not shown), via the through-holes for the retentate ports in permeate/filtrate member 6220 and membrane 6024 (also not shown). FIG. 6H depicts a cover 6044 that is configured to be disposed upon the top of reservoir assembly 6050. Cover 6044 has round cut-outs at the top of retentate reservoirs 6052 and permeate/filtrate reservoir 6054. Again, at the bottom of retentate reservoirs 6052 fluid channels 6034 can be seen, where fluid channels 6034 fluidically couple retentate reservoirs 6052 with the retentate ports 6028 (not shown). Also shown are three pneumatic ports 6030 for each retentate reservoir 6052 and permeate/filtrate reservoir 6054. FIG. 6I depicts a gasket 6045 that is configures to be disposed upon the cover 6044 of reservoir assembly 6050. Seen are three fluid transfer ports 6042 for each retentate reservoir 6052 and for permeate/filtrate reservoir 6054. Again, three pneumatic ports 6030, for each retentate reservoir 6052 and for permeate/filtrate reservoir 6054, are shown.

FIG. 6J depicts an exploded view of a TFF module 6000. Seen are components reservoir assembly 6050, a cover 6044 to be disposed on reservoir assembly 6050, a gasket 6045 to be disposed on cover 6044, retentate member 6022, membrane or filter 6024, and permeate/filtrate member 6020. Also seen is permeate/filtrate port 6026, which mates with permeate/filtrate port 6026 on permeate/filtrate reservoir 6054, as well as two retentate ports 6028, which mate with retentate ports 6028 on retentate reservoirs 6052 (where only one retentate reservoir 6052 can be seen clearly in this FIG. 6J). Also seen are through-holes for retentate ports 6028 in membrane 6024 and permeate/filtrate member 6020.

FIG. 6K depicts an embodiment of assembled TFF module 6000. Note that in this embodiment of a TFF module the retentate member 6022 is no longer “upper”, and the permeate/filtrate member 6020 is no longer “lower”, as the retentate member 6022 and permeate/filtrate member 6020 are coupled side-to-side with membrane 6024 sandwiched between retentate member 6022 and permeate/filtrate member 6020. Also, retentate member 6022, membrane member 6024, and permeate/filtrate member 6020 are coupled side-to-side with reservoir assembly 6050. Seen are two retentate ports 6028 (which couple the tangential flow channel 6002 in retentate member 6022 to the two retentate reservoirs (not shown), and one permeate/filtrate port 6026, which couples the tangential flow channel 6002 in permeate/filtrate member 6020 to the permeate/filtrate reservoir (not shown). Also seen is tangential flow channel 6002, which is formed by the mating of retentate member 6022 and permeate/filtrate member 6020, with membrane 6024 sandwiched between and bifurcating tangential flow channel 6002. Also seen is energy director 6091, which in this FIG. 6K has been used to ultrasonically weld or couple retentate member 6022 and permeate/filtrate member 6020, surrounding membrane 6024. Cover 6044 can be seen on top of reservoir assembly 6050, and gasket 6045 is disposed upon cover 6044. Gasket 6045 engages with and provides a fluid-tight seal and pneumatic connections with fluid transfer ports 6042 and pneumatic ports 6030, respectively. FIG. 6J also shows the length, height, and width dimensions of the TFF module 6000. The assembled TFF device 6000 typically is from 50 to 175 mm in height, or from 75 to 150 mm in height, or from 90 to 120 mm in height; from 50 to 175 mm in length, or from 75 to 150 mm in length, or from 90 to 120 mm in length; and is from 30 to 90 mm in depth, or from 40 to 75 mm in depth, or from about 50 to 60 mm in depth. An exemplary TFF device is 110 mm in height, 120 mm in length, and 55 mm in depth.

Like in other embodiments described herein, the TFF device or module depicted in FIGS. 6E-6K can constantly measure cell culture growth, and in some aspects cell culture growth is measured via optical density (OD) of the cell culture in one or both of the retentate reservoirs and/or in the flow channel of the TFF device. Optical density may be measured continuously (kinetic monitoring) or at specific time intervals; e.g., every 5, 10, 15, 20, 30 45, or 60 seconds, or every 1, 2, 3, 4, 5, 6, 7, 8, 9, or 10 or so on minutes. Further, the TFF module can adjust growth parameters (temperature, aeration) to have the cells at a desired optical density at a desired time.

FIG. 6L is an exemplary pneumatic block diagram suitable for the TFF module depicted in FIGS. 6E-6K. The pump is connected to two solenoid valves (SV5 and SV6) delivering positive pressure (P) or negative pressure (V). The two solenoid valves SV5 and SV6 couple the pump to the manifold, and two solenoid valves, SV1 and SV2, are connected to the reservoirs (RR1 and RR2). There are also two solenoid valves in reserve (SV3 and SV4). There is a proportional valve (PV2 and PV2), a flow meter (FM1 and FM2), and a pressure sensor (Pressure Sensors 1 and 2) positioned in between each of solenoid valves SV1 and SV2 connecting the pump to the system and the solenoid valves SV1 And SV2 to the reservoirs. The pressure sensors and prop valves work in concert in a feedback loop to maintain the required pressure.

As an alternative to the TFF module described above, a cell concentration module comprising a hollow filter may be employed. Examples of filters suitable for use in the present invention include membrane filters, ceramic filters and metal filters. The filter may be used in any shape; the filter may for example be cylindrical or essentially flat. Preferably, the filter used is a membrane filter, preferably a hollow fiber filter. The term “hollow fiber” is meant a tubular membrane. The internal diameter of the tube is at least 0.1 mm, more preferably at least 0.5 mm, most preferably at least 0.75 mm and preferably the internal diameter of the tube is at most 10 mm, more preferably at most 6 mm, most preferably at most 1 mm. Filter modules comprising hollow fibers are commercially available from various companies, including G.E. Life Sciences (Marlborough, Mass.) and InnovaPrep (Drexel, Mo.). Specific examples of hollow fiber filter systems that can be used, modified or adapted for use in the present methods and systems include, but are not limited to, U.S. Pat. Nos. 9,738,918; 9,593,359; 9,574,977; 9,534,989; 9,446,354; 9,295,824; 8,956,880; 8,758,623; 8,726,744; 8,677,839; 8,677,840; 8,584,536; 8,584,535; and 8,110,112.

### Transformation Module

FIGS. 7A-7E depict variations on one embodiment of a cell transformation module (in this case, a flow-through electroporation device) that may be included in a cell growth/concentration/transformation instrument. FIGS. 7A and 7B are top perspective and bottom perspective views, respectively, of six co-joined flow-through electroporation devices 750. FIG. 7A depicts six flow-through electroporation units 750 arranged on a single substrate 756. Each of the six flow-through electroporation units 750 have inlet wells 752 that define cell sample inlets and outlet wells 754 that define cell sample outlets. FIG. 7B is a bottom perspective view of the six co-joined flow-through electroporation devices of FIG. 7A also depicting six flow-through electroporation units 750 arranged on a single substrate 756. Six inlet wells 752 can be seen, one for each flow-through electroporation unit 750, and one outlet well 754 can be seen (the outlet well of the left-most flow-through electroporation unit 750). Additionally seen in FIG. 7B are an inlet 702, outlet 704, flow channel 706 and two electrodes 708 on either side of a constriction in flow channel 706 in each flow-through electroporation unit 750. Once the six flow-through electroporation units 750 are fabricated, they can be snapped apart and used one at a time, or alternatively in embodiments where two or more flow-through electroporation units 750 can be used in parallel without separation.

The flow-through electroporation devices 750 achieve high efficiency cell electroporation with low toxicity. The flow-through electroporation devices 750 of the disclosure allow for particularly easy integration with robotic liquid handling instrumentation that is typically used in automated systems such as air displacement pipettors. Such automated instrumentation includes, but is not limited to, off-the-shelf automated liquid handling systems from Tecan (Mannedorf, Switzerland), Hamilton (Reno, Nev.), Beckman Coulter (Fort Collins, Colo.), etc.

Generally speaking, microfluidic electroporation—using cell suspension volumes of less than approximately 10 mL and as low as 1 μl—allows more precise control over a transfection or transformation process and permits flexible integration with other cell processing tools compared to bench-scale electroporation devices. Microfluidic electroporation thus provides unique advantages for, e.g., single cell transformation, processing and analysis; multi-unit electroporation device configurations; and integrated, automatic, multi-module cell processing and analysis.

In specific embodiments of the flow-through electroporation devices 750 of the disclosure, the toxicity level of the transformation results in greater than 10% viable cells after electroporation, preferably greater than 15%, 20%, 25%, 30%, 35%, 40%, 45%, 50%, 55%, 60%, 70%, 75%, 80%, 85%, 90%, or even 95% viable cells following transformation, depending on the cell type and the nucleic acids being introduced into the cells.

The flow-through electroporation device 750 described in relation to FIGS. 7A-7E comprises a housing with an electroporation chamber, a first electrode and a second electrode configured to engage with an electric pulse generator, by which electrical contacts engage with the electrodes of the electroporation device 750. In certain embodiments, the electroporation devices are autoclavable and/or disposable, and may be packaged with reagents in a reagent cartridge. The electroporation device 750 may be configured to electroporate cell sample volumes between 1 μl to 2 mL, 10 μl to 1 mL, 25 μl to 750 μl, or 50 μl to 500 μl. The cells that may be electroporated with the disclosed electroporation devices 850 include mammalian cells (including human cells), plant cells, yeasts, other eukaryotic cells, bacteria, archaea, and other cell types.

In one exemplary embodiment, FIG. 7C depicts a top view of a flow-through electroporation device 750 having an inlet 702 for introduction of cells and an exogenous reagent to be electroporated into the cells (“cell sample”) and an outlet 704 for the cell sample following electroporation. Electrodes 708 are introduced through electrode channels (not shown in this FIG. 7C) in the device. FIG. 7D shows a cutaway view from the top of flow-through electroporation device 750, with the inlet 702, outlet 704, and electrodes 708 positioned with respect to a constriction in flow channel 706. A side cutaway view of a lower portion of flow-through electroporation device 750 in FIG. 7E illustrates that electrodes 708 in this embodiment are positioned in electrode channels 710 and perpendicular to flow channel 706 such that the cell sample flows from the inlet channel 712 through the flow channel 706 to the outlet channel 714, and in the process the cell sample flows into the electrode channels 710 to be in contact with electrodes 708. In this aspect, the inlet channel 712, outlet channel 714 and electrode channels 710 all originate from the top planar side of the device; however, the flow-through electroporation architecture depicted in FIGS. 7C-7E is but one architecture useful with the reagent cartridges described herein. Additional electrode architectures are described, e.g., in U.S. Ser. No. 16/147,120, filed 24 Sep. 2018; Ser. No. 16/147,865, filed 30 Sep. 2018; and Ser. No. 16/147,871, filed 30 Sep. 2018.

### Exemplary Workflows

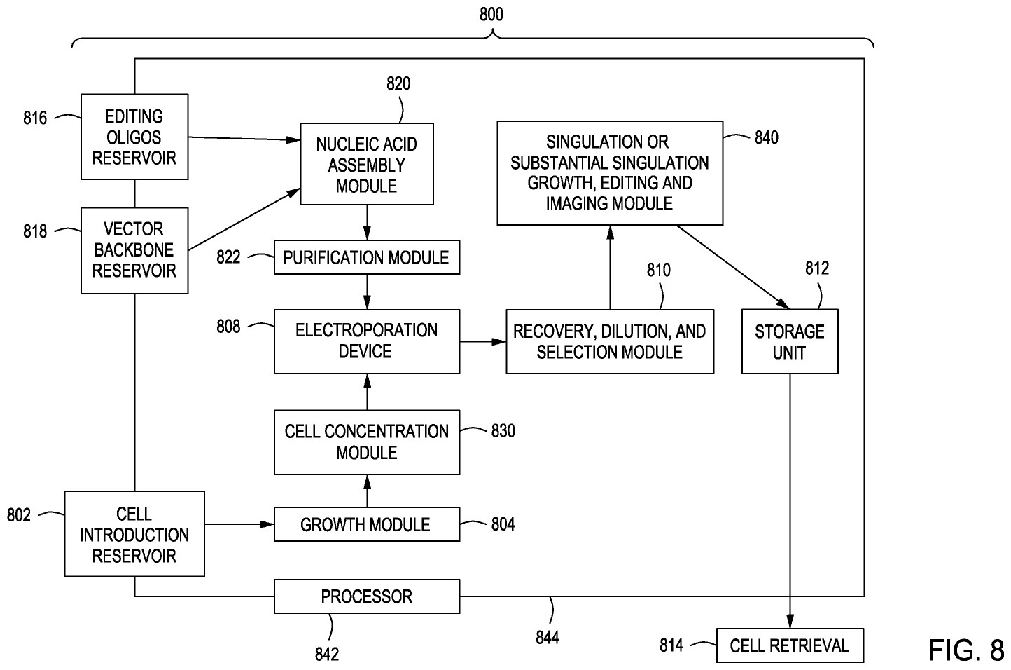

FIG. 8 is a simplified block diagram of an embodiment of an exemplary automated multi-module cell processing instrument comprising an isolation or substantial isolation/incubation/editing and normalization or cherry-picking module for enrichment or selection of edited cells. The cell processing instrument 800 may include a housing 844, a reservoir of cells to be transformed or transfected 802, and a growth module (a cell growth device) 804. The cells to be transformed are transferred from a reservoir to the growth module to be cultured until the cells hit a target OD. Once the cells hit the target OD, the growth module may cool or freeze the cells for later processing, or the cells may be transferred to a cell concentration module 830 where the cells are rendered electrocompetent and concentrated to a volume optimal for cell transformation. Exemplary cell concentration devices of use in the automated multi-module cell processing system include those described in U.S. Ser. No. 16/561,701, filed 5 Sep. 2019, which is incorporated by reference in its entirety. Once concentrated, the cells are then transferred to the electroporation device 808 (e.g., transformation/transfection module, with one exemplary module described above in relation to FIGS. 7A-7E).

Exemplary electroporation devices of use in the automated multi-module cell processing instruments include flow-through electroporation devices such as those described in U.S. Ser. No. 16/147,120, filed 28 Sep. 2018; Ser. No. 16/147,353, filed 28 Sep. 2018; Ser. No. 16/147,865, filed 30 Sep. 2018; and Ser. No. 16/147,871, filed 30 Sep. 2018 all of which are herein incorporated by reference in their entirety.

In addition to the reservoir for storing the cells 830, the automated multi-module cell processing instrument 800 may include a reservoir for storing editing oligonucleotide cassettes 816 and a reservoir for storing an expression vector backbone 818. Both the editing oligonucleotide cassettes and the expression vector backbone are transferred from a reagent cartridge to a nucleic acid assembly module 820, where the editing oligonucleotide cassettes are inserted into the expression vector backbone. The assembled nucleic acids may be transferred into an optional purification module 822 for desalting and/or other purification and/or concentration procedures needed to prepare the assembled nucleic acids for transformation. Alternatively, pre-assembled nucleic acids, e.g., an editing vector, may be stored within reservoir 816 or 818. Once the processes carried out by the purification module 822 are complete, the assembled nucleic acids are transferred to, e.g., an electroporation device 808, which already contains the cell culture grown to a target OD and rendered electrocompetent via cell concentration module 830. In electroporation device 908, the assembled nucleic acids are introduced into the cells. Following electroporation, the cells are transferred into a combined recovery/selection module 810. For examples of multi-module cell editing instruments, see U.S. Pat. No. 10,253,316, filed 30 Jun. 2018; U.S. Pat. No. 10,329,559, filed 7 Feb. 2019; and U.S. Pat. No. 10,323,242, filed 7 Feb. 2019; U.S. Pat. No. 10,421,959, filed 14 May 2019; and U.S. Ser. No. 16/412,195, filed 14 May 2019; and Ser. No. 16/423,289, filed 28 May 2019, all of which are herein incorporated by reference in their entirety.

Following recovery, and, optionally, selection, the cells are transferred to an isolation or substantial isolation, editing, and growth module 840, where the cells are diluted and compartmentalized such that there is an average of substantially one cell per compartment. Once substantially or largely isolated, the cells are allowed to grow for a pre-determined number of doublings. Once these initial colonies are established, editing proceeds and the edited cells are grown to establish colonies, which are grown to terminal size (e.g., the colonies are normalized). In some embodiments, editing is induced by one or more of the editing components, preferably the gRNA, being under the control of an inducible promoter. In some embodiments, the inducible promoter is activated by a rise in temperature and deactivated by lowering the temperature. Similarly, in embodiments where the solid wall device comprises a filter forming the bottom of the microwell, the solid wall device can be transferred to a plate (e.g., an agar plate or even to liquid medium) comprising a medium with a component that activates or induces editing, then transferred to a medium that deactivates editing. In solid wall devices such as those described herein, induction of editing and deactivation of editing can take place by media exchange. Subsequent to editing, media exchange may be performed once again for medium containing a fluorescent moiety such as a fluorescent antibody capable of phenotypically identifying cells that have been edited properly. Cells that exhibit the desired phenotype can be selected or “cherry picked” or cells that do not exhibit the desired phenotype can be ablated. If cells without the desired phenotype are ablated, the desired cells can be pooled.

The recovery, selection, and isolation/incubation/editing and normalization modules may all be separate, may be arranged and combined as shown in FIG. 8, or may be arranged or combined in other configurations. In certain embodiments, all of recovery, selection, isolation or substantial isolation, growth (e.g., incubation), editing, and normalization are performed in a solid wall device described in relation to FIGS. 2A and 2 and 3A-3I. Alternatively, recovery, selection, and dilution, if necessary, are performed in liquid medium in a separate vessel such as in a rotating growth vial (module), then transferred to the isolation/incubation/editing and normalization module.

Once the normalized cell colonies are pooled, and the cells may be stored, e.g., in a storage unit or module 812, where the cells can be kept at, e.g., 4° C. until the cells are retrieved for further study 814. Alternatively, the cells may be used in another round of editing. The multi-module cell processing instrument 800 is controlled by a processor 842 configured to operate the instrument based on user input, as directed by one or more scripts, or as a combination of user input or a script. The processor 842 may control the timing, duration, temperature, and operations of the various modules of the instrument 800 and the dispensing of reagents. For example, the processor 842 may cool the cells post-transformation until editing is desired, upon which time the temperature may be raised to a temperature conducive of genome editing and cell growth. The processor may be programmed with standard protocol parameters from which a user may select, a user may specify one or more parameters manually or one or more scripts associated with the reagent cartridge may specify one or more operations and/or reaction parameters. In addition, the processor may notify the user (e.g., via an application to a smart phone or other device) that the cells have reached the target OD as well as update the user as to the progress of the cells in the various modules in the multi-module system.

The automated multi-module cell processing instrument 800 is a nuclease-directed genome editing system and can be used in single editing systems (e.g., introducing one or more edits to a cellular genome in a single editing process).

It should be apparent to one of ordinary skill in the art given the present disclosure that the process described may be recursive and multiplexed; that is, cells may go through the workflow described in relation to FIG. 8, then the resulting edited culture may go through another (or several or many) rounds of additional editing (e.g., recursive editing) with different editing vectors. For example, the cells from round 1 of editing may be diluted and an aliquot of the edited cells edited by editing vector A may be combined with editing vector B, an aliquot of the edited cells edited by editing vector A may be combined with editing vector C, an aliquot of the edited cells edited by editing vector A may be combined with editing vector D, and so on for a second round of editing. After round two, an aliquot of each of the double-edited cells may be subjected to a third round of editing, where, e.g., aliquots of each of the AB-, AC-, AD-edited cells are combined with additional editing vectors, such as editing vectors X, Y, and Z. That is to say that double-edited cells AB may be combined with and edited by vectors X, Y, and Z to produce triple-edited edited cells ABX, ABY, and ABZ; double-edited cells AC may be combined with and edited by vectors X, Y, and Z to produce triple-edited cells ACX, ACY, and ACZ; and double-edited cells AD may be combined with and edited by vectors X, Y, and Z to produce triple-edited cells ADX, ADY, and ADZ, and so on. In this process, many permutations and combinations of edits can be executed, leading to very diverse cell populations and cell libraries. In any recursive process, it is advantageous to “cure” the previous engine and editing vectors (or single engine+editing vector in a single vector system). “Curing” is a process in which one or more vectors used in the prior round of editing is eliminated from the transformed cells. Curing can be accomplished by, e.g., cleaving the vector(s) using a curing plasmid thereby rendering the editing and/or engine vector (or single, combined vector) nonfunctional; diluting the vector(s) in the cell population via cell growth (that is, the more growth cycles the cells go through, the fewer daughter cells will retain the editing or engine vector(s)), or by, e.g., utilizing a heat-sensitive origin of replication on the editing or engine vector (or combined engine+editing vector). The conditions for curing will depend on the mechanism used for curing; that is, in this example, how the curing plasmid cleaves the editing and/or engine plasmid. For details of curing protocols useful in the present methods, modules and instruments, see U.S. Ser. No. 62/857,967, filed 6 Jun. 2019.

While this invention is satisfied by embodiments in many different forms, as described in detail in connection with preferred embodiments of the invention, it is understood that the present disclosure is to be considered as exemplary of the principles of the invention and is not intended to limit the invention to the specific embodiments illustrated and described herein. Numerous variations may be made by persons skilled in the art without departure from the spirit of the invention. The scope of the invention will be measured by the appended claims and their equivalents. The abstract and the title are not to be construed as limiting the scope of the present invention, as their purpose is to enable the appropriate authorities, as well as the general public, to quickly determine the general nature of the invention. In the claims that follow, unless the term “means” is used, none of the features or elements recited therein should be construed as means-plus-function limitations pursuant to 35 U.S.C. § 112, ¶6.

TABLE 1
SWIIN Valve Status and Prop Valve PSI

| | | | | | | | | | | | Prop | Prop |
|---|---|---|---|---|---|---|---|---|---|---|---|---|
| | | Manifold | Manifold | | | | SV1 | SV2 | SV3 | SV4 | Valve | Valve |
| Description of step | Step | ARM 1 | ARM 2 | Pump | SV-POS | SV-NEG | (RR1) | (PR1) | (PR2) | (RR2) | 1 (psi) | 2 (psi) |
| Load SWIIN cartridge | 1 | open | open | 0 | 0 | 0 | 0 | 0 | 0 | 0 | 0 | 0 |
| on the instrument | | | | | | | | | | | | |
| Transfer 10 mL | 2 | open | closed | 0 | 0 | 0 | 0 | 0 | 0 | 0 | 0 | 0 |
| PBS-0.01% Tween80 | | | | | | | | | | | | |
| from Reagent | | | | | | | | | | | | |
| Strip to PR1 | | | | | | | | | | | | |
| Load PBS-0.01% | 3 | closed | sealed | 1 | 1 | 0 | 0 | 1 | 0 | 0 | 0.5 | 0.5 |
| Tween80 into Permeate | | | | | | | | | | | | |
| channel - Bubble Flush | | | | | | | | | | | | |
| Load PBS-0.01% | 4 | closed | sealed | 1 | 1 | 0 | 1 | 1 | 0 | 1 | 0.5 | 0.5 |
| Tween80 into Permeate | | | | | | | | | | | | |
| channel - Fill Channel | | | | | | | | | | | | |
| Flood Retentate - | 5 | sealed | sealed | 1 | 0 | 1 | 1 | 0 | 0 | 1 | −0.7 | −0.7 |
| Symmetrically Apply | | | | | | | | | | | | |
| Vacuum to Retentate | | | | | | | | | | | | |
| Flood Retentate - Sweep | 6 | sealed | sealed | 1 | 0 | 1 | 0 | 0 | 0 | 1 | 0 | −0.7 |
| to RR2 | | | | | | | | | | | | |
| Aspirate liquid out of | 7 | open | open | 1 | 0 | 0 | 0 | 0 | 0 | 0 | 0 | 0 |
| RR1 & RR2 | | | | | | | | | | | | |
| Transfer 9.5 mL of | 8 | open | closed | 1 | 0 | 0 | 0 | 0 | 0 | 0 | 0 | 0 |
| PBS-0.01% Tween80 | | | | | | | | | | | | |
| from Reagent Strip | | | | | | | | | | | | |
| to RR1 | | | | | | | | | | | | |
| Transfer 0.5 mL cell | 9 | open | closed | 1 | 0 | 0 | 0 | 0 | 0 | 0 | 0 | 0 |
| Solution from FTEP | | | | | | | | | | | | |
| to RR1 | | | | | | | | | | | | |
| Pipette cell solution | 10 | open | closed | 1 | 0 | 0 | 0 | 0 | 0 | 0 | 0 | 0 |
| up/down in RR1 | | | | | | | | | | | | |
| Pull cell solution | 11 | open | sealed | 1 | 0 | 1 | 0 | 0 | 0 | 1 | 0 | −0.7 |
| from RR1 into | | | | | | | | | | | | |
| Retentate Channel | | | | | | | | | | | | |
| Pull retentate | 12 | sealed | sealed | 1 | 0 | 1 | 0 | 1 | 1 | 0 | −0.7 | −0.7 |
| through membrane | | | | | | | | | | | | |
| (low vac) | | | | | | | | | | | | |
| Pull retentate | 13 | sealed | sealed | 1 | 0 | 1 | 0 | 1 | 1 | 0 | −1 | −1 |
| through membrane | | | | | | | | | | | | |
| (high vac) | | | | | | | | | | | | |
| Sweep all fluid to PR1 | 14 | sealed | sealed | 1 | 1 | 0 | 1 | 0 | 1 | 1 | 0.5 | .05 |
| Aspirate liquid out of | 15 | open | open | 1 | 0 | 0 | 0 | 0 | 0 | 0 | 0 | 0 |
| PR1 & PR2 | | | | | | | | | | | | |
| Transfer 10 mL media | 16 | open | closed | 1 | 0 | 0 | 0 | 0 | 0 | 0 | 0 | 0 |
| from Reagent Strip to | | | | | | | | | | | | |
| PR1 | | | | | | | | | | | | |
| Load media from PR1 | 17 | open | closed | 1 | 0 | 1 | 0 | 0 | 1 | 0 | 0 | −0.7 |
| into Permeate | | | | | | | | | | | | |
| channel | | | | | | | | | | | | |
| Aspirate liquid out of | 18 | open | open | 0 | 0 | 0 | 0 | 0 | 0 | 0 | 0 | 0 |
| PR1 & PR2 | | | | | | | | | | | | |
| INCUBATE SWIIN | 19 | closed | closed | 0 | 0 | 0 | 0 | 0 | 0 | 0 | 0 | 0 |
| 30 C. #1 - may | | | | | | | | | | | | |
| require intermittent | | | | | | | | | | | | |
| airflow, media rinses | | | | | | | | | | | | |
| Ramp up (30 C. to | 20 | closed | closed | 0 | 0 | 0 | 0 | 0 | 0 | 0 | 0 | 0 |
| 42 C.) | | | | | | | | | | | | |
| INCUBATE SWIIN | 21 | closed | closed | 0 | 0 | 0 | 0 | 0 | 0 | 0 | 0 | 0 |
| 42 C. - may require | | | | | | | | | | | | |
| intermittent airflow, | | | | | | | | | | | | |
| media rinses | | | | | | | | | | | | |
| Ramp down (42 C. to | 22 | closed | closed | 0 | 0 | 0 | 0 | 0 | 0 | 0 | 0 | 0 |
| 30 C.) | | | | | | | | | | | | |
| INCUBATE SWIIN | 23 | closed | closed | 0 | 0 | 0 | 0 | 0 | 0 | 0 | 0 | 0 |
| 30 C. #2 - may | | | | | | | | | | | | |
| require intermittent | | | | | | | | | | | | |
| airflow, media rinses | | | | | | | | | | | | |
| Pull media out of | 24 | sealed | sealed | 1 | 0 | 1 | 0 | 0 | 1 | 0 | 0 | −0.7 |
| Permeate channel | | | | | | | | | | | | |
| into PR2 | | | | | | | | | | | | |
| Aspirate liquid | 25 | open | open | 1 | 0 | 0 | 0 | 0 | 0 | 0 | 0 | 0 |
| out of PR2 | | | | | | | | | | | | |
| Transfer 10 mL | 26 | open | closed | 1 | 0 | 0 | 0 | 0 | 0 | 0 | 0 | 0 |
| media + 10% glycerol | | | | | | | | | | | | |
| from Reagent Strip to | | | | | | | | | | | | |
| PR1 | | | | | | | | | | | | |
| Pull media + 10% | 27 | open | sealed | 1 | 0 | 1 | 0 | 0 | 1 | 0 | 0 | −0.7 |
| glycerol from PR1 | | | | | | | | | | | | |
| into Permeate | | | | | | | | | | | | |
| channel | | | | | | | | | | | | |
| Flood | 28 | sealed | sealed | 1 | 1 | 0 | 0 | 1 | 1 | 0 | 1 | 1 |
| Retentate - Dislodge | | | | | | | | | | | | |
| Cells | | | | | | | | | | | | |
| Sweep all fluid | 29 | sealed | sealed | 1 | 0 | 1 | 0 | 0 | 0 | 1 | 0 | −0.7 |
| to RR2 | | | | | | | | | | | | |
| Aspirate 5 mL cell | 30 | closed | open | 0 | 0 | 0 | 0 | 0 | 0 | 0 | 0 | 0 |
| solution from RR2 | | | | | | | | | | | | |
| into final vial | | | | | | | | | | | | |
| Aspirate liquid out of | 31 | open | open | 0 | 0 | 0 | 0 | 0 | 0 | 0 | 0 | 0 |
| RR1 & RR2 | | | | | | | | | | | | |
| Unload SWIIN | 32 | open | open | 0 | 0 | 0 | 0 | 0 | 0 | 0 | 0 | 0 |

TABLE 2
SWIIN Flow Meter Status

| | FM1 | FM1 | FM2 | FM2 | | | |
|---|---|---|---|---|---|---|---|
| | (PR2) | (PR2) | (RR2) | (RR2) | Valve delay | Requires | Duration |
| Description of step | Detection | Threshold | Detection | Threshold | after spike(s) | pLLD | (sec) |
| Load SWIIN cartridge | NA | NA | NA | NA | NA | 0 | N/A |
| on the instrument | | | | | | | |
| Transfer 10 mL | NA | NA | NA | NA | NA | 0 | as |
| PBS-0.01% Tween80 | | | | | | | needed |
| from Reagent Strip | | | | | | | |
| to PR1 | | | | | | | |
| Load PBS-0.01% | NA | NA | NA | NA | NA | 0 | 0.5 |
| Tween80 into | | | | | | | |
| Permeate | | | | | | | |
| channel - Bubble | | | | | | | |
| Flush | | | | | | | |
| Load PBS-0.01% | NA | NA | FALL | 10 | 5 | 0 | until FM |
| Tween80 into | | | | | | | trigger |
| Permeate | | | | | | | |
| channel - Fill | | | | | | | |
| Channel | | | | | | | |
| Flood Retentate - | NA | NA | NA | NA | NA | 0 | 30 |
| Symmetrically | | | | | | | |
| Apply Vacuum to | | | | | | | |
| Retentate | | | | | | | |
| Flood Retentate - | NA | NA | NA | NA | NA | 0 | 60 |
| Sweep to RR2 | | | | | | | |
| Aspirate liquid out of | NA | NA | NA | NA | NA | 0 | determined |
| RR1 & RR2 | | | | | | | by ADP |
| Transfer 9.5 mL of | NA | NA | NA | NA | NA | 0 | determined |
| PBS-0.01% Tween80 | | | | | | | by ADP |
| from Reagent Strip | | | | | | | |
| to RR1 | | | | | | | |
| Transfer 0.5 mL | NA | NA | NA | NA | NA | 0 | determined |
| cell Solution | | | | | | | by ADP |
| from FTEP to RR1 | | | | | | | |
| Pipette cell solution | NA | NA | NA | NA | NA | 0 | 10 |
| up/down in RR1 | | | | | | | |
| Pull cell solution | NA | NA | NA | NA | NA | 1 | until |
| from RR1 into | | | | | | | RR1 & RR2 |
| Retentate Channel | | | | | | | are equal |
| | | | | | | | volume |
| Pull retentate | NA | NA | NA | NA | NA | 0 | 90 |
| through membrane | | | | | | | |
| (low vac) | | | | | | | |
| Pull retentate | NA | NA | NA | NA | NA | 0 | 30 |
| through membrane | | | | | | | |
| (high vac) | | | | | | | |
| Sweep all fluid | RISE | 50 | NA | NA | 0 | 0 | until FM |
| to PR1 | | | | | | | trigger |
| Aspirate liquid out of | NA | NA | NA | NA | NA | 0 | determined |
| PR1 & PR2 | | | | | | | by ADP |
| Transfer 10 mL | NA | NA | NA | NA | NA | 0 | determined |
| media from Reagent | | | | | | | by ADP |
| Strip to PR1 | | | | | | | |
| Load media from | NA | NA | NA | NA | NA | 1 | until PR1 |
| PR1 into Permeate | | | | | | | is nearly |
| channel | | | | | | | exhausted |
| Aspirate liquid out of | NA | NA | NA | NA | NA | 0 | determined |
| PR1 & PR2 | | | | | | | by ADP |
| INCUBATE SWIIN | NA | NA | NA | NA | NA | 0 | 32400 |
| 30 C. #1 - may | | | | | | | |
| require intermittent | | | | | | | |
| airflow, media rinses | | | | | | | |
| Ramp up (30 C. | NA | NA | NA | NA | NA | 0 | 900 |
| to 42 C.) | | | | | | | |
| INCUBATE SWIIN | NA | NA | NA | NA | NA | 0 | 7200 |
| 42 C. - may | | | | | | | |
| require intermittent | | | | | | | |
| airflow, media rinses | | | | | | | |
| Ramp down (42 C. to 30 C.) | NA | NA | NA | NA | NA | 0 | 900 |
| INCUBATE SWIIN | NA | NA | NA | NA | NA | 0 | 32400 |
| 30 C. #2 - may | | | | | | | |
| require intermittent | | | | | | | |
| airflow, media rinses | | | | | | | |
| Pull media out of | RISE | 50 | NA | NA | 0 | 0 | until FM |
| Permeate channel | | | | | | | trigger |
| into PR2 | | | | | | | |
| Aspirate liquid | NA | NA | NA | NA | NA | 0 | determined |
| out of PR2 | | | | | | | by ADP |
| Transfer 10 mL | NA | NA | NA | NA | NA | 0 | determined |
| media + 10% | | | | | | | by ADP |
| glycerol from | | | | | | | |
| Reagent Strip to PR1 | | | | | | | |
| Pull media + 10% | NA | NA | NA | NA | NA | 1 | until |
| glycerol from PR1 | | | | | | | PR1 and PR2 |
| into Permeate channel | | | | | | | are equal |
| | | | | | | | volume |
| Flood Retentate - | NA | NA | NA | NA | NA | 0 | 30 |
| Dislodge Cells | | | | | | | |
| Sweep all fluid to RR2 | NA | NA | RISE | 50 | 0 | 0 | until FM |
| | | | | | | | trigger |
| Aspirate 5 mL cell | NA | NA | NA | NA | NA | 0 | determined |
| solution from RR2 | | | | | | | by ADP |
| into final vial | | | | | | | |
| Aspirate liquid out of | NA | NA | NA | NA | NA | 0 | determined |
| RR1 & RR2 | | | | | | | by ADP |
| Unload SWIIN | NA | NA | NA | NA | NA | 0 | N/A |

TABLE 3
SWIIN Reservoir Volumes
Volumes (Assume: SWIIN Volume = 5 mL)

| | RR1 | RR1 | PR1 | PR1 | PR2 | PR2 | RR2 | RR2 | Temperature | |
|---|---|---|---|---|---|---|---|---|---|---|
| Description of step | Initial | Final | Initial 2 | Final 2 | Initial 3 | Final 3 | Initial 4 | Final 4 | (° C.) | Notes |
| Load SWIIN cartridge | 0.0 | 0.0 | 0.0 | 0.0 | 0.0 | 0.0 | 0.0 | 0.0 | Continue | |
| on the instrument | | | | | | | | | Ramp Up | |
| | | | | | | | | | (RT to 30) | |
| Transfer 10 mL | 0.0 | 0.0 | 10.0 | 10.0 | 0.0 | 0.0 | 0.0 | 0.0 | Continue | |
| PBS-0.01% Tween80 | | | | | | | | | Ramp Up | |
| from Reagent Strip | | | | | | | | | (RT to 30) | |
| to PR1 | | | | | | | | | | |
| Load PBS-0.01% | 0.0 | 0.0 | 10.0 | 9.8 | 0.0 | 0.0 | 0.0 | 0.0 | Continue | This 0.5 s step |
| Tween80 into | | | | | | | | | Ramp Up | consumes very |
| Permeate | | | | | | | | | (RT to 30) | little liquid |
| channel - Bubble | | | | | | | | | | |
| Flush | | | | | | | | | | |
| Load PBS-0.01% | 0.0 | 0.0 | 9.8 | 2.5 | 0.0 | 2.5 | 0.0 | 0.0 | Continue | Requires debounce |
| Tween80 into | | | | | | | | | Ramp Up | delay for flow |
| Permeate | | | | | | | | | (RT to 30) | sensor to reach |
| channel - Fill | | | | | | | | | | high, trigger |
| Channel | | | | | | | | | | threshold untested |
| Flood Retentate - | 0.0 | 2.5 | 2.5 | 0.0 | 2.5 | 0.0 | 0.0 | 2.5 | Continue | |
| Symmetrically | | | | | | | | | Ramp Up | |
| Apply Vacuum to | | | | | | | | | (RT to 30) | |
| Retentate | | | | | | | | | | |
| Flood Retentate - | 2.5 | 0.0 | 0.0 | 0.0 | 0.0 | 0.0 | 2.5 | 10.0 | Continue | |
| Sweep to RR2 | | | | | | | | | Ramp Up | |
| | | | | | | | | | (RT to 30) | |
| Aspirate liquid | 0.0 | 0.0 | 0.0 | 0.0 | 0.0 | 0.0 | 10.0 | 0.0 | Continue | |
| out of | | | | | | | | | Ramp Up | |
| RR1 & RR2 | | | | | | | | | (RT to 30) | |
| Transfer 9.5 mL of | 0.0 | 9.5 | 0.0 | 0.0 | 0.0 | 0.0 | 0.0 | 0.0 | Continue | |
| PBS-0.01% Tween80 | | | | | | | | | Ramp Up | |
| from Reagent Strip | | | | | | | | | (RT to 30) | |
| to RR1 | | | | | | | | | | |
| Transfer 0.5 mL cell | 0.0 | 10.0 | 0.0 | 0.0 | 0.0 | 0.0 | 0.0 | 0.0 | Continue | |
| Solution from FTEP | | | | | | | | | Ramp Up | |
| to RR1 | | | | | | | | | (RT to 30) | |
| Pipette cell solution | 0.0 | 10.0 | 0.0 | 0.0 | 0.0 | 0.0 | 0.0 | 0.0 | Continue | |
| up/down in RR1 | | | | | | | | | Ramp Up | |
| | | | | | | | | | (RT to 30) | |
| Pull cell solution | 10.0 | 2.5 | 0.0 | 0.0 | 0.0 | 0.0 | 0.0 | 2.5 | Continue | |
| from RR1 into | | | | | | | | | Ramp Up | |
| Retentate Channel | | | | | | | | | (RT to 30) | |
| Pull retentate | 2.5 | 0.0 | 0.0 | 2.5 | 0.0 | 2.5 | 2.5 | 0.0 | Continue | |
| through membrane | | | | | | | | | Ramp Up | |
| (low vac) | | | | | | | | | (RT to 30) | |
| Pull retentate | 0.0 | 0.0 | 2.5 | 2.5 | 2.5 | 2.5 | 0.0 | 0.0 | Continue | This step is only |
| through membrane | | | | | | | | | Ramp Up | here as a safeguard, |
| (high vac) | | | | | | | | | (RT to 30) | all liquid should |
| | | | | | | | | | | have transferred |
| | | | | | | | | | | in prev step |
| Sweep all fluid | 0.0 | 0.0 | 2.5 | 10.0 | 2.5 | 0.0 | 0.0 | 0.0 | Continue | |
| to PR1 | | | | | | | | | Ramp Up | |
| | | | | | | | | | (RT to 30) | |
| Aspirate liquid | 0.0 | 0.0 | 10.0 | 0.0 | 0.0 | 0.0 | 0.0 | 0.0 | Continue | |
| out of | | | | | | | | | Ramp Up | |
| PR1 & PR2 | | | | | | | | | (RT to 30) | |
| Transfer 10 mL | 0.0 | 0.0 | 0.0 | 10.0 | 0.0 | 0.0 | 0.0 | 0.0 | Continue | |
| media from Reagent | | | | | | | | | Ramp Up | |
| Strip to PR1 | | | | | | | | | (RT to 30) | |
| Load media from | 0.0 | 0.0 | 10.0 | 0.5 | 0.0 | 4.5 | 0.0 | 0.0 | Continue | |
| PR1 into Permeate | | | | | | | | | Ramp Up | |
| channel | | | | | | | | | (RT to 30) | |
| Aspirate liquid | 0.0 | 0.0 | 0.5 | 0.0 | 4.5 | 0.0 | 0.0 | 0.0 | Continue | May keep some media |
| out of | | | | | | | | | Ramp Up | in PR1/PR2 reservoirs |
| PR1 & PR2 | | | | | | | | | (RT to 30) | during incubation |
| INCUBATE SWIIN | 0.0 | 0.0 | 0.0 | 0.0 | 0.0 | 0.0 | 0.0 | 0.0 | 30 | 9 hours; may |
| 30 C. #1 - may | | | | | | | | | | intermittently |
| require intermittent | | | | | | | | | | seal manifold arms |
| airflow, media rinses | | | | | | | | | | for airflow, media |
| | | | | | | | | | | rinses |
| Ramp up (30 C. to | 0.0 | 0.0 | 0.0 | 0.0 | 0.0 | 0.0 | 0.0 | 0.0 | Ramp Up | 15 minutes; Ramp |
| 42 C.) | | | | | | | | | (30 to 42) | rate still being |
| | | | | | | | | | | worked by G8 |
| INCUBATE SWIIN | 0.0. | 0.0 | 0.0 | 0.0 | 0.0 | 0.0 | 0.0 | 0.0 | 42 | 2 hours; may |
| 42 C. - may | | | | | | | | | | intermittently |
| require intermittent | | | | | | | | | | seal manifold |
| airflow, media rinses | | | | | | | | | | arms for airflow, |
| | | | | | | | | | | media rinses |
| Ramp down (42 C. to | 0.0 | 0.0 | 0.0 | 0.0 | 0.0 | 0.0 | 0.0 | 0.0 | Ramp Down | 15 minutes; Ramp |
| 30 C.) | | | | | | | | | (42 to 30) | rate still being |
| | | | | | | | | | | worked on by G8 |
| INCUBATE SWIIN 30 C. | 0.0 | 0.0 | 0.0 | 0.0 | 0.0 | 0.0 | 0.0 | 0.0 | 30 | 9 hours; may |
| #2 - may require | | | | | | | | | | intermittently |
| intermittent airflow, | | | | | | | | | | seal manifold |
| media rinses | | | | | | | | | | arms for airflow, |
| | | | | | | | | | | media rinses |
| Pull media out of | 0.0 | 0.0 | 0.0 | 0.0 | 0.0 | 10.0 | 0.0 | 0.0 | Ramp Down | |
| Permeate channel | | | | | | | | | (30 to RT) | |
| into PR2 | | | | | | | | | | |
| Aspirate liquid | 0.0 | 0.0 | 0.0 | 0.0 | 0.0 | 0.0 | 0.0 | 0.0 | Continue | |
| out of PR2 | | | | | | | | | Ramp Down | |
| | | | | | | | | | (30 to RT) | |
| Transfer 10 mL | 0.0 | 0.0 | 0.0 | 10.0 | 0.0 | 0.0 | 0.0 | 0.0 | Continue | |
| media + 10% | | | | | | | | | Ramp Down | |
| glycerol from Reagent | | | | | | | | | (30 to RT) | |
| Strip to PR1 | | | | | | | | | | |
| Pull media + 10% | 0.0 | 0.0 | 10.0 | 2.5 | 0.0 | 2.5 | 0.0 | 0.0 | Continue | |
| glycerol from PR1 | | | | | | | | | Ramp Down | |
| into Permeate channel | | | | | | | | | (30 to RT) | |
| Flood Retentate - | 0.0 | 2.5 | 2.5 | 0.0 | 2.5 | 0.0 | 0.0 | 2.5 | Continue | |
| Dislodge Cells | | | | | | | | | Ramp Down | |
| | | | | | | | | | (30 to RT) | |
| Sweep all fluid | 2.5 | 0.0 | 0.0 | 0.0 | 0.0 | 0.0 | 0.0 | 10.0 | Continue | |
| to RR2 | | | | | | | | | Ramp Down | |
| | | | | | | | | | (30 to RT) | |
| Aspirate 5 mL cell | 0.0 | 0.0 | 0.0 | 0.0 | 0.0 | 0.0 | 0.0 | 5.0 | Continue | |
| solution from RR2 | | | | | | | | | Ramp Down | |
| into final vial | | | | | | | | | (30 to RT) | |
| Aspirate liquid | 0.0 | 0.0 | 0.0 | 0.0 | 0.0 | 0.0 | 0.0 | 0.0 | Continue | |
| out of | | | | | | | | | Ramp Down | |
| RR1 & RR2 | | | | | | | | | (30 to RT) | |
| Unload SWIIN | 0.0 | 0.0 | 0.0 | 0.0 | 0.0 | 0.0 | 0.0 | 0.0 | Continue | |
| | | | | | | | | | Ramp Down | |
| | | | | | | | | | (30 to RT) | |

We claim:

1. A method for determining a phenotype of edited cells on an automated solid wall isolation, incubation and normalization (SWIIN) device comprising:
providing a SWIIN device comprising:
a retentate member comprising:
an upper surface and a lower surface and a first and second end,
an upper portion of a serpentine channel defined by raised areas on the lower surface of the retentate member, wherein the upper portion of the serpentine channel traverses the lower surface of the retentate member for about 50% to about 90% of the length and width of the lower surface of the retentate member;
at least retentate one port fluidically connected to the upper portion of the serpentine channel; and
a reservoir cover at the first end of the retentate member;
a permeate member disposed under the retentate member comprising:
an upper surface and a lower surface and a first and second end,
a lower portion of a serpentine channel defined by raised areas on the upper surface of the permeate member, wherein the lower portion of the serpentine channel traverses the upper surface of the permeate member for about 50% to about 90% of the length and width of the upper surface of the permeate member, and wherein the lower portion of the serpentine channel is configured to mate with the upper portion of the serpentine channel to form a mated serpentine channel;
at least one permeate port fluidically connected to the lower portion of the serpentine channel; and
a first and second reservoir at the first end of the permeate member, wherein the first reservoir is fluidically connected to the at least one retentate port in the retentate member and the second reservoir is fluidically connected to the at least one permeate port in the permeate member;
a perforated member comprising perforations defining wells disposed under and adjacent to the retentate member; and
a gasket disposed on top of the reservoir cover of the retentate member, wherein the gasket comprises for each reservoir a reservoir access aperture configured to provide fluid access to a reservoir and a pneumatic access aperture configured to provide pneumatic access to a reservoir;
distributing cells into the wells defined by the perforated member wherein the cells are distributed in a Poisson or substantial Poisson distribution and wherein the cells comprise a promoter driving expression of a nuclease and a promoter driving transcription of a gRNA and a donor DNA;
growing the cells;
providing conditions to allow the cells to be edited by the nuclease, gRNA and donor DNA;
performing medium exchange in the SWIIN device to provide a fluorescent screening reagent agent in a medium; and
imaging the cells.

2. The method of claim 1, wherein the imaging of the cells comprises a fluorescent excitation source, light display optics, light assortment optics, and detection and visualization.

3. The method of claim 2, wherein the imaging of the cells further comprises light filtration.

4. The method of claim 1, wherein the cells are bacterial cells.

5. The method of claim 1, wherein the cells are yeast cells.

6. The method of claim 1, wherein the cells are mammalian cells.

7. The method of claim 1, wherein the fluorescent screening agent is a fluorescently-labeled antibody.

8. The method of claim 1, further comprising, after the step of imaging, the steps of washing the cells in the wells and cherry-picking or selecting cells in wells that fluoresce.

9. The method of claim 1, further comprising, after the step of imaging, the steps of washing the cells in the wells and ablating cells in wells that do not fluoresce.

10. The method of claim 1, wherein the SWIIN device is part of an automated multi-module cell editing instrument.

11. The method of claim 10, wherein the automated multi-module cell editing instrument further comprises a growth module for growing the cells and a transformation module for transforming the cells.

12. The method of claim 1, wherein the nuclease is MAD7.

13. The method of claim 1, wherein the nuclease is under the control of an inducible promoter.

14. The method of claim 1, wherein the gRNA and donor DNA are covalently-linked in an editing cassette.

15. The method of claim 1, wherein transcription of the gRNA and donor DNA is under the control of an inducible promoter.

16. The method of claim 1, further comprising the step of measuring a baseline fluorescence prior to the step of performing medium exchange.

17. The method of claim 1, wherein the fluorescent screening agent is a mixture of several fluorescently-labeled antibodies.

18. The method of claim 1, further comprising the step of delivering an antigen to the cells prior to medium exchange, and the fluorophore is a fluorescently-labeled secondary polyclonal antibody.

19. The method of claim 1, wherein the cells are distributed in a substantial Poisson distribution in the wells.

20. The method of claim 19, wherein less than 50 cells are distributed per well.

21. The method of claim 20, wherein less than 20 cells are distributed per well.

22. The method of claim 21, wherein less than 10 cells are distributed per well.

23. The method of claim 1, wherein the cells are distributed in a Poisson distribution.
